(19) United States
(12) Patent Application Publication
(10) Pub. No.: US 2024/0409530 A1
(43) Pub. Date: Dec. 12, 2024

(54) Disubstituted Pyrimidine Compounds for Ketohexokinase Inhibition

(71) Applicant: Centennial Therapeutics, LLC, Centennial, CO (US)

(72) Inventors: Gavin Whitlock, South San Francisco, CA (US); Paul Glossop, South San Francisco, CA (US); Timothy P. Rolph, Brookline, MA (US); Erik J. Tillman, Arlington, MA (US)

(21) Appl. No.: 18/634,193

(22) Filed: Apr. 12, 2024

Related U.S. Application Data
(60) Provisional application No. 63/495,649, filed on Apr. 12, 2023.

(51) Int. Cl. C07D 403/14; A61K 31/506

(52) U.S. Cl. CPC C07D 403/14; A61K 31/506

(57) ABSTRACT

Disclosed herein are methods of inhibiting ketohexokinase (KHK) in a biological sample or subject, and of treating or preventing diseases or disorders (e.g., diseases or disorders associated with KHK dysregulation), comprising administering to said biological sample or subject an effective amount of a compound represented by Formula I or a compound of Table A, or a pharmaceutically acceptable salt thereof.

### FIELD OF THE DISCLOSURE

This disclosure relates generally to novel ketohexokinase (KHK) inhibitor compounds, pharmaceutical compositions thereof, and the use of these compounds for the treatment of conditions such as non-alcoholic fatty liver disease (NAFLD), metabolic dysfunction-associated steatohepatitis (MASH), metabolic dysfunction-associated steatotic liver disease (MASLD), non-alcoholic steatohepatitis (NASH), hypertriglyceridemia, hypercholesterolemia, type 2 diabetes mellitus (T2D), diabetic kidney disease (DKD), alcoholic steatohepatitis (ASH), addictive craving including sugar or alcohol craving or alcohol use disorder, neurodegenerative diseases such as Parkinson's Disease or Alzheimer's Disease, hyperuricemia, gout, or cancer.

### BACKGROUND

KHK (also called ketohexokinase or fructokinase) catalyzes the first step in fructose metabolism, phosphorylating fructose to fructose-1-phophate (F1P) and depleting intracellular ATP. There is no negative feedback mechanism by which F1P inhibits KHK metabolism of fructose, therefore accumulation of F1P is directly related to the amount of fructose either (1) transported into the cell via the GLUT transporters or (2) formed intracellularly from glucose via the polyol pathway, and metabolized via KHK. The accumulation of F1P and depletion of ATP cause deleterious consequences in cells and tissue, including oxidative stress, osmolar stress, endothelial dysfunction, and metabolic dysregulation. Responses to these insults include lipogenesis, hyperuricemia and gluconeogenesis, which drive metabolic diseases including metabolic syndrome and its sequelae.

Two isoforms of KHK exist: KHK-A, which is ubiquitously expressed but has a lower affinity for fructose, and KHK-C, which is preferentially expressed in liver, kidney, brain, and intestine with a much higher affinity for fructose. While KHK-C drives the majority of physiological flux from fructose to F1P, KHK-A may compensate for downregulation, inhibition, or deletion of KHK-C, particularly as intracellular fructose concentrations rise. Humans with genetic polymorphisms in KHK have the benign phenotype Essential Fructosuria and do not accumulate F1P, whereas the more serious condition Hereditary Fructose Intolerance is caused by a polymorphism in the ALDOB gene encoding aldolase B, which catalyzes the second step in fructose metabolism, leading to deleterious accumulation of F1P and increased oxidative stress caused by depletion of the intracellular adenine nucleotide pool.

Increased consumption of fructose and fructose-containing polysaccharides like sucrose and high-fructose corn syrup is associated with the rise in metabolic disorders including obesity, insulin resistance, type 2 diabetes, dyslipidemia, metabolic liver diseases including NASH and other liver diseases associated with increased hepatocyte stress including alpha-1 antitrypsin deficiency and hemochromatosis. Targeting this first, rate-limiting step of fructose metabolism, i.e., phosphorylation by KHK, has been suggested to be a promising therapeutic strategy for these metabolic disorders as well as other diseases driven by fructose metabolism: cancers, neurodegenerative disorders including Parkinson's disease and Alzheimer's disease, addictive cravings for sugar and alcohol, hyperuricemia, and other complications of insulin resistance including diabetic retinopathy and diabetic kidney disease.

Attempts to inhibit KHK have thus far stalled in preclinical or clinical development, with only modest inhibition of fructose uptake and metabolism demonstrated by the furthest-developed compound, PF-06835919, in phase 1 and phase 2 clinical studies. While this molecule demonstrated potent inhibition of the isolated KHK-C enzyme, its potency was reduced approximately 10-fold in a cell-based assay of KHK activity, translating to the need for twice-daily 300 mg doses in the phase 2 clinical study. Even with these high doses, only modest effects on liver fat and markers of liver injury were observed. As a carboxylic acid, PF-06835919 is a substrate of organic anion transporter protein, leading to increased accumulation in liver relative to other tissues and organs. While KHK-C is preferentially expressed in liver, it is also highly expressed in kidney, intestine and some brain centers, other sites of fructose metabolism to F1P, which may drive metabolic dysregulation, endothelial dysfunction, metabolic disease or craving disorders. Further, PF-06835919 is a KHK-C-biased inhibitor, with much weaker inhibition of KHK-A. PF-06835919 is disclosed in US 2017/0183328 A1. See also, J. Med. Chem. 2020, 63, 13546-13560. In addition, U.S. Pat. No. 11,124,500 discloses certain disubstituted pyrazole KHK inhibitor compounds. Also, Durham et al. J. Med Chem. 2023, 66, 15960-15976 “Identification of LY3522348: A Highly Selective and Orally Efficacious Ketohexokinase Inhibitor”, discloses ketohexokinase inhibitors. While KHK-C drives fructose metabolism in several tissues at physiological fructose concentrations, as fructose concentrations rise (due to diet, metabolic disease, or inhibition of KHK-C), the contribution of KHK-A metabolism of fructose to F1P becomes more substantial.

Therefore, there is significant need to develop a KHK inhibitor that is potent both in enzyme assays and in cell-based assays; provides balanced inhibition of KHK-C and KHK-A to minimize escape flux to F1P through KHK-A or compensation by KHK-A; and is systemically distributed to inhibit KHK-C and KHK-A in not just liver, but also kidney, intestine, and all other tissues.

### SUMMARY

The present disclosure generally relates to methods of inhibiting ketohexokinase (KHK), to methods of treating or preventing diseases or disorders in a subject (e.g., wherein the diseases or disorders are associated with KHK dysregulation or fructose metabolism, including secondary to excessive consumption of fructose and/or alcohol), and to compounds and compositions that can be employed for such methods.

The disclosure provides compounds of Formula I, and pharmaceutically acceptable salts thereof:

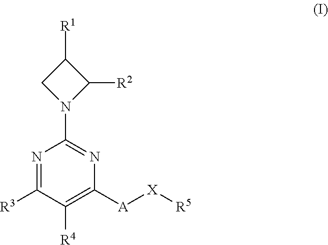

wherein R¹ is H or OH; R² is C₁₋₆alkyl or C₁₋₆haloalkyl; R³ is C₁₋₆alkyl or C₁₋₆haloalkyl; R⁴ is H, halo, CN, C₁₋₆alkyl, C_{1- 6}alkoxy, or C₃₋₅cycloalkyl; A is a 5-membered heteroaryl comprising 2-3 nitrogen ring atoms; X is a bond or C₁₋₆alkyleneC(O); and R⁵ is 4- to 6-membered heterocycloalkyl having 1 or 2 ring nitrogen atoms, wherein the heterocycloalkyl is optionally substituted with 1 or 2 C₁₋₆alkyl, with the proviso that when R⁴ is H, compounds where both R² is methyl and R³ is trifluoromethyl are excluded. In embodiments, A is pyrazolyl. In embodiments, the compounds have the structure of Formula Ix:

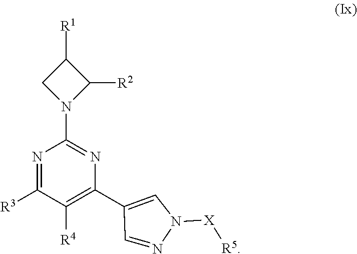

Further provided are methods of administering to a biological sample or patient a safe and effective amount of a compound as disclosed herein, e.g., as represented by Formula I or a compound of Table A.

Also provided herein are methods of inhibiting ketohexokinase (KHK) in a biological sample or in a patient (e.g., in a cell) by administering to said biological sample or patient an effective amount of a compound as disclosed herein, e.g., as represented by Formula I or a compound of Table A.

Further provided are methods of treating or preventing diseases or disorders in a subject (e.g., wherein the diseases or disorders are associated with KHK dysregulation or fructose metabolism, including secondary to excessive consumption of fructose and/or alcohol), comprising administering to said subject an effective amount of a compound as disclosed herein, e.g., as represented by Formula I or a compound of Table A.

Also provided are pharmaceutical compositions comprising a compound as disclosed herein, e.g., as represented by Formula I or a compound of Table A, or a pharmaceutically acceptable salt thereof, and a pharmaceutically acceptable excipient, carrier, adjuvant or vehicle.

Also provided are uses of a compound described herein for inhibiting ketohexokinase (KHK), e.g., in a cell, and for treating or preventing diseases or disorders in a subject (e.g., wherein the diseases or disorders are associated with KHK dysregulation or fructose metabolism, including secondary to excessive consumption of fructose and/or alcohol).

Further provided herein are uses of a compound described herein for the manufacture of a medicament for inhibiting ketohexokinase (KHK), and for treating or preventing diseases or disorders in a subject (e.g., wherein the diseases or disorders are associated with KHK dysregulation or fructose metabolism, including via excessive consumption of fructose and/or alcohol).

### BRIEF DESCRIPTION OF THE DRAWINGS

FIG. 1 shows an XPRD diffractogram comparison between A21 Free Base (top trace) and A21 HCl salt (bottom trace).

FIG. 2 shows an ¹H NMR spectrum comparison between A21 Free Base and HCl salt (DMSO-d6).

### DETAILED DESCRIPTION

Provided herein are compounds, and their use in inhibiting ketohexokinase (KHK), e.g., in a cell, and for treating or preventing diseases or disorders in a subject (e.g., wherein the diseases or disorders are associated with KHK dysregulation or fructose metabolism, including secondary to excessive consumption of fructose and/or alcohol). Also provided are uses of the compounds described herein, or pharmaceutically acceptable salts thereof, or pharmaceutically acceptable compositions comprising such a compound or a pharmaceutically acceptable salt thereof, for inhibiting ketohexokinase (KHK), e.g., in a cell, and for treating or preventing diseases or disorders in a subject (e.g., wherein the diseases or disorders are associated with KHK dysregulation).

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this disclosure, unless only one of the isomers is specifically indicated. Therefore, single stereochemical isomers as well as enantiomeric, diastereomeric, cis/trans, conformational, and rotational mixtures of the present compounds are within the scope of the disclosure. In some cases, the compounds disclosed herein are stereoisomers. “Stereoisomers” refer to compounds that differ in the chirality of one or more stereocenters. Stereoisomers include enantiomers and diastereomers. The compounds disclosed herein can exist as a single stereoisomer, or as a mixture of stereoisomers. Stereochemistry of the compounds shown herein indicate a relative stereochemistry, not absolute, unless discussed otherwise. As indicated herein, a single stereoisomer, diastereomer, or enantiomer refers to a compound that is at least more than 50% of the indicated stereoisomer, diastereomer, or enantiomer, and in some cases, at least 90% or 95% of the indicated stereoisomer, diastereomer, or enantiomer.

Unless otherwise indicated, all tautomeric forms of the compounds of the disclosure are within the scope of the disclosure.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this disclosure. Such compounds are useful, for example, as analytical tools or probes in biological assays. Such compounds, especially deuterium analogs, can also be therapeutically useful.

The compounds of the disclosure are defined herein by their chemical structures and/or chemical names. Where a compound is referred to by both a chemical structure and a chemical name, and the chemical structure and chemical name conflict, the chemical structure is determinative of the compound's identity.

### Compounds

Provided herein are compounds of Formula I, and pharmaceutically acceptable salts thereof:

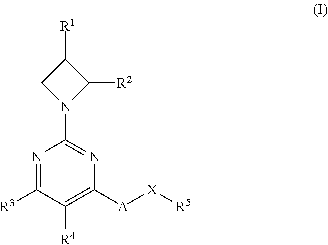

wherein
R¹ is H or OH;
R² is C₁₋₆alkyl or C₁₋₆haloalkyl;
R³ is C₁₋₆alkyl or C₁₋₆haloalkyl;
R⁴ is H, halo, CN, C₁₋₆alkyl, C₁₋₆alkoxy, or C₃₋₅cycloalkyl;
A is a 5-membered heteroaryl comprising 2-3 nitrogen ring atoms;
X is a bond or C₁₋₆alkylene-C(O); and
R⁵ is 4- to 6-membered heterocycloalkyl having 1 or 2 ring nitrogen atoms, wherein the heterocycloalkyl is optionally substituted with 1 or 2 C₁₋₆alkyl, with the proviso that when R⁴ is H, compounds where both R² is methyl and R³ is trifluoromethyl are excluded.

In some cases, A is a 5-membered heteroaryl comprising 2 nitrogen ring atoms. In some cases, A is a 5-membered heteroaryl comprising 3 nitrogen ring atoms. In some cases, A is pyrazolyl. In some cases, the compound or salt has the structure of Formula Ix:

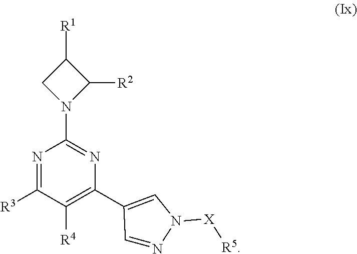

In some cases, X is a bond. In some cases, X is C₁₋₆alkylene-C(O). In some cases, X is CH₂—C(O). In some cases, X—R⁵ is C(O)—C₁₋₆alkylene-R⁵.

In some cases, R⁵ is 4-membered heterocycloalkyl having 1 ring nitrogen atom wherein the heterocycloalkyl is optionally substituted with 1 or 2 C₁₋₆alkyl. In some cases, R⁵ is 5-membered heterocycloalkyl having 1 or 2 ring nitrogen atoms, wherein the heterocycloalkyl is optionally substituted with 1 or 2 C₁₋₆alkyl. In some cases, R⁵ is 6-membered heterocycloalkyl having 1 or 2 ring nitrogen atoms, wherein the heterocycloalkyl is optionally substituted with 1 or 2 C₁₋₆alkyl. In some cases, R⁵ is 4-membered heterocycloalkyl having 1 ring nitrogen atom, wherein the heterocycloalkyl is optionally substituted with 1 or 2 C₁₋₆alkyl. In some cases, R⁵ is 5-membered heterocycloalkyl having 1 ring nitrogen atom, wherein the heterocycloalkyl is optionally substituted with 1 or 2 C₁₋₆alkyl. In some cases, R⁵ is 5-membered heterocycloalkyl having 2 ring nitrogen atoms, wherein the heterocycloalkyl is optionally substituted with 1 or 2 C₁₋₆alkyl. In some cases, R⁵ is 6-membered heterocycloalkyl having 2 ring nitrogen atoms, wherein the heterocycloalkyl is optionally substituted with 1 or 2 C₁₋₆alkyl. In some cases, R⁵ is azetidinyl or piperazinyl, and is optionally substituted with 1 or 2 C₁₋₆alkyl. In some cases, R⁵ is azetidinyl, and is optionally substituted with 1 or 2 C₁₋₆alkyl. In some cases, R⁵ is piperazinyl, and is optionally substituted with 1 or 2 C₁₋₆alkyl. In some cases, R⁵ is unsubstituted. In some cases, R⁵ is substituted with 1 C₁₋₆alkyl. In some cases, R⁵ is substituted with 1 methyl. In some cases, R⁵ is substituted with 2 C₁₋₆alkyl.

In some cases, the compound has the structure of Formula Ia or Ib:

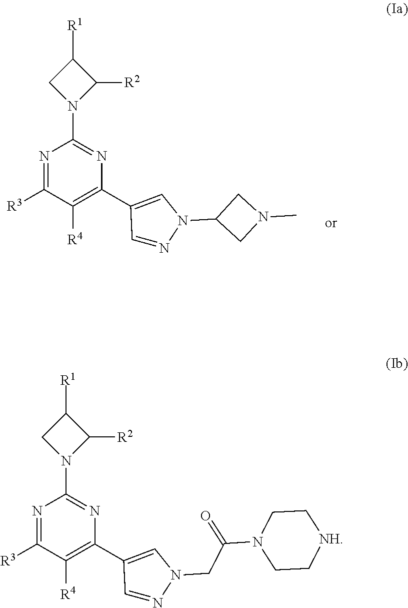

In some cases, the compound has the structure of Formula Ia:

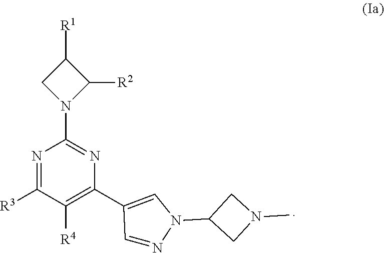

In some cases, the compound has the structure of Ib:

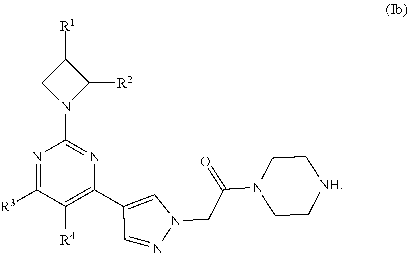

In some cases, the compound of Formula I has the structure of Formula II:

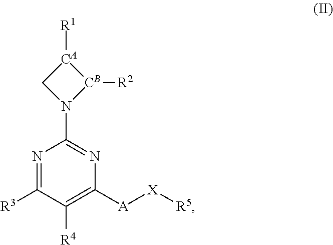

wherein C^{A} and C^{B} represent carbon stereocenters having the same or opposite stereochemistry. For C^{A} to be carbon stereocenter, R¹ cannot be H. As such, for compounds of Formula (II), R¹ is OH. In some cases, the compound has a structure of Formula (IIx):

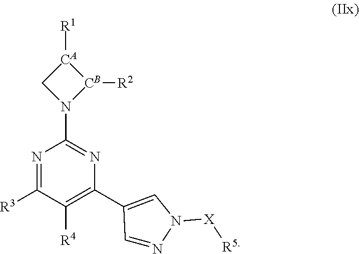

In some cases, the compound of Formula I has the structure of Formula IIa or (IIb):

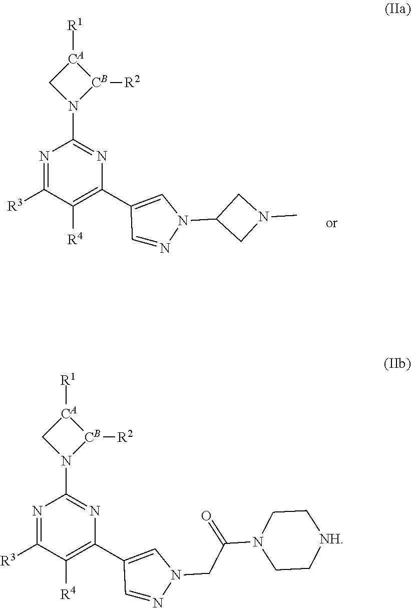

In some cases, C^{A} and C^{B} represent carbon stereocenters having the same stereochemistry. In some cases, C^{A} and C^{B} represent carbon stereocenters having opposite stereochemistry. In some cases, C^{A} is a carbon in the R configuration and C^{B} is a carbon in the S configuration. In some cases, C^{A} is a carbon in the S configuration and C^{B} is a carbon in the R configuration.

In some cases, R¹ is H. In some cases, R¹ is OH.

In some cases, R² is C₁₋₆alkyl. In some cases, R² is methyl. In some cases, R² is C₁₋₆haloalkyl. In some cases, R² is C₁haloalkyl. In some cases, R² is CHF₂ or CF₃. In some cases, R² is CHF₂. In some cases, R² is CF₃. In some cases, R² is methyl, CHF₂, or CF₃.

In some cases, R³ is C₁₋₆alkyl. In some cases, R³ is methyl. In some cases, R³ is C₁₋₆haloalkyl. In some cases, R³ is C₁haloalkyl. In some cases, R³ is CHF₂ or CF₃. In some cases, R³ is CHF₂. In some cases, R³ is CF₃. In some cases, R³ is methyl, CHF₂, or CF₃.

In some cases, R⁴ is H. In some cases, R⁴ is halo. In some cases, R⁴ is F or Cl. In some cases, R⁴ is F. In some cases, R⁴ is Cl. In some cases, R⁴ is C₁₋₆alkyl. In some cases, R⁴ is methyl or ethyl. In some cases, R⁴ is methyl. In some cases, R³ is CHF₂ and R⁴ is methyl. In some cases, R⁴ is ethyl. In some cases, R⁴ is C₁₋₆alkoxy. In some cases, R⁴ is methoxy. In some cases, R⁴ is C₃₋₅cycloalkyl. In some cases, R⁴ is cyclopropyl. In some cases, R⁴ is methyl, ethyl, methoxy, F, Cl, CHF₂, CF₃, or cyclopropyl.

In some cases, R¹ is H or hydroxy, R² is methyl, X is a bond and R⁵ is a 4- to 6-membered heterocycloalkyl having 1 ring nitrogen atom, wherein the heterocycloalkyl is optionally substituted with one C₁₋₂alkyl. In some cases, X is C₁₋₆alkylene-C(O), R⁵ is azetidinyl optionally substituted with 1 or 2 C₁₋₆alkyl, R¹ is H or hydroxy and R² is C₁₋₃alkyl.

Preferred compounds are compounds wherein R³ is CHF₂, R⁴ is methyl, R¹ is hydroxy and R² is methyl.

The proviso that “when R⁴ is H, compounds where both R² is methyl and R³ is trifluoromethyl are excluded” means that any individual compound where both R² is methyl and R³ is trifluoromethyl is not included in the claims.

As used herein, the term “alkyl” or “alkylene” means a saturated straight or branched chain hydrocarbon. The term Cₙ means the alkyl group has “n” carbon atoms. For example, C₄alkyl refers to an alkyl group that has 4 carbon atoms. C₁₋₆alkyl refers to an alkyl group having a number of carbon atoms encompassing the entire range (i.e., 1 to 6 carbon atoms), as well as all subgroups (e.g., 1-6, 2-6, 1-5, 2-6, 1-4, 2-5, 1, 2, 3, 4, 5, and 6 carbon atoms). Specific examples include, but are not limited to, methyl, ethyl, isopropyl, n-propyl, sec-butyl, t-butyl, n-pentyl, and n-hexyl. The claim term C₁₋₆alkylene-C(O) is defined as being attached to R⁵ through the C(O) moiety.

As used herein, the terms “halogen” and “halo” mean F, Cl, Br, or I.

As used herein, the term “haloalkyl” refers to an alkyl group substituted with one or more halogen substituents. For example, C₁-C₆haloalkyl refers to a C₁-C₆ alkyl group substituted with one or more halogen atoms, e.g., 1, 2, 3, 4, 5, or 6 halogen atoms. Non-limiting examples of haloalkyl groups include fluoromethyl, difluoromethyl, trifluoromethyl, chloromethyl, dichloromethyl, and trichloromethyl groups. Similarly, haloalkoxy refers to an alkoxy group substituted with one or more halogen atoms e.g., 1, 2, 3, 4, 5, or 6 halogen atoms.

The term “alkoxy” used herein refers to an —O-alkyl group.

The term “cycloalkyl” refers to a non-aromatic monocyclic, fused, bridged or spiro ring system whose ring atoms are carbon and which can be saturated or have one or more units of unsaturation. The cycloalkyl can have three to five ring carbon atoms. Specific examples include, but are not limited to, cyclopentyl, cyclopropyl, and cyclobutyl. A cycloalkyl ring is unsubstituted or substituted as described herein.

The term “heterocycloalkyl” as used herein refers to a non-aromatic monocyclic, fused, spiro or bridged ring system which can be saturated or contain one or more units of unsaturation, having five to eight ring atoms in which one or more (e.g., one to three, or one, two, or three) ring atoms is a heteroatom selected from, N, S, and O. An “N-heterocyle” indicates that at least one of the ring heteroatoms is a nitrogen atom. In some embodiments, the heterocycloalkyl comprises 4 to 6 ring members. In some embodiments, the heterocyle comprises 4 ring members. In some embodiments, the heterocycloalkyl comprises 6 ring members. Examples of heterocycloalkyls include, but are not limited to, oxetanyl, azetidinyl, thietanyl, piperidinyl, piperazinyl, pyrrolidinyl, pyrazolidinyl, imidazolidinyl, oxazolidinyl, isoxazolidinyl, thiazolidinyl, isothiazolidinyl, tetrahydrofuranyl, tetrahydrothiophenyl, morpholino (including, for example, 3-morpholino, 4-morpholino), 2-thiomorpholino, 3-thiomorpholino, 4-thiomorpholino, 1-pyrrolidinyl, 2-pyrrolidinyl, 3-pyrrolidinyl, pyrrolidin-2-one, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, dihydrofuranyl, 1,3 dioxolanyl, 1,4-dioxanyl, 1,3-oxathiol, oxathianyl, 1,3-dithianyl, 1,4-oxathiolanyl, 1,4-oxathianyl, 1,4-dithianyl, thiomorpholinyl, tetrahydropyranyl, dihydropyranyl, and 1,3-dihydro-imidazol-2-onyl. A heterocycoalkyl ring is unsubstituted or substituted as described herein.

The terms “heteroaryl” refers to a heterocycle that is aromatic, having five members. Heteroaryl groups have two or three ring nitrogen (N) heteroatoms. Examples of heteroaryl groups include imidazolyl, pyrazolyl, and triazolyl (e.g., 1H-1,2,3-triazolyl or 4H-1,2,4-triazolyl). A heteroaryl ring is unsubstituted or substituted as described herein.

As described herein, compounds of the disclosure may optionally be substituted with one or more substituents, such as illustrated generally, or as exemplified by particular classes, subclasses, and species of the disclosure. It will be appreciated that the phrase “optionally substituted” is used interchangeably with the phrase “substituted or unsubstituted.” In general, the term “substituted”, whether preceded by the term “optionally” or not, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position.

Specific compounds contemplated include compounds in the following Tables. Compounds showing particular stereocenters indicate at least a relative stereoisomerism. Compounds having a chiral center without indication of a particular stereoisomerism indicate a mixture of stereocenters at that chiral center.

The compound can be a compound as listed in Table A, or a pharmaceutically acceptable salt thereof. The compounds in Table A were prepared according to methods described in the Examples section and other methods known to those skilled in the art.

TABLE A

| | Name | Structure |
|---|---|---|
| A1 | 2-(4-{2-[(R)-2-(trifluoromethyl)-1- azetidinyl]-5-methyl-6- (trifluoromethyl)-4-pyrimidinyl}- 1-pyrazolyl)-1-(1-piperazinyl)-1- ethanone | 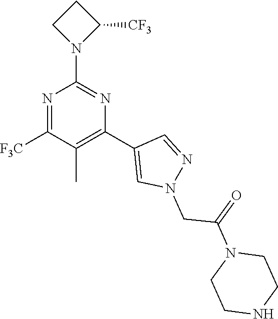 [SMILES: Cc1c(-c2cnn(CC(=O)N3CCNCC3)c2)nc(N2CC[C@@H]2C(F)(F)F)nc1C(F)(F)F] |
| A2 | 2-(4-{2-[(R)-2-(trifluoromethyl)-1- azetidinyl]-6-(trifluoromethyl)-4- pyrimidinyl}-1-pyrazolyl)-1-(1- piperazinyl)-1-ethanone | 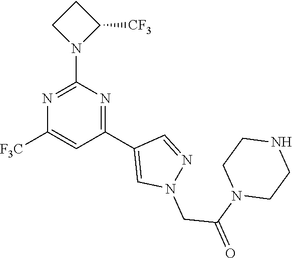 [SMILES: O=C(Cn1cc(-c2cc(C(F)(F)F)nc(N3CC[C@@H]3C(F)(F)F)n2)cn1)N1CCNCC1] |
| A3 | 2-(4-{2-[(S)-2-methyl-1- azetidinyl]-5-methyl-6- (trifluoromethyl)-4-pyrimidinyl}- 1-pyrazolyl)-1-(1-piperazinyl)-1- ethanone | 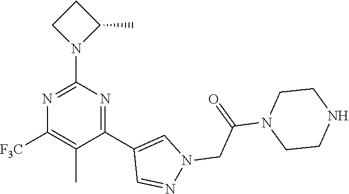 [SMILES: Cc1c(-c2cnn(CC(=O)N3CCNCC3)c2)nc(N2CC[C@@H]2C)nc1C(F)(F)F] |
| A4 | 2-(4-{2-[(S)-2-methyl-1- azetidinyl]-5-chloro-6- (trifluoromethyl)-4-pyrimidinyl}- 1-pyrazolyl)-1-(1-piperazinyl)-1- ethanone | 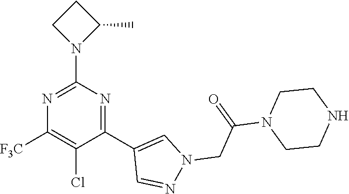 [SMILES: C[C@H]1CCN1c1nc(-c2cnn(CC(=O)N3CCNCC3)c2)c(Cl)c(C(F)(F)F)n1] |
| A5 | 2-(4-{2-[(S)-2-methyl-1- azetidinyl]-5-methoxy-6- (trifluoromethyl)-4-pyrimidinyl}- 1-pyrazolyl)-1-(1-piperazinyl)-1- ethanone | 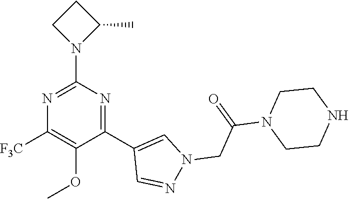 [SMILES: COc1c(-c2cnn(CC(=O)N3CCNCC3)c2)nc(N2CC[C@@H]2C)nc1C(F)(F)F] |
| A6 | 2-(4-{2-[(S)-2-methyl-1- azetidinyl]-5-ethyl-6- (trifluoromethyl)-4-pyrimidinyl}- 1-pyrazolyl)-1-(1-piperazinyl)-1- ethanone | 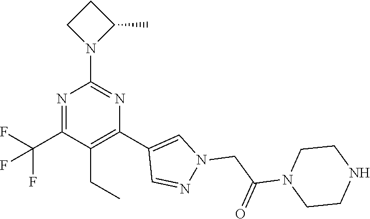 [SMILES: CCc1c(-c2cnn(CC(=O)N3CCNCC3)c2)nc(N2CC[C@@H]2C)nc1C(F)(F)F] |
| A7 | 2-(4-{2-[(S)-2-methyl-1- azetidinyl]-6-(difluoromethyl)-4- pyrimidinyl}-1-pyrazolyl)-1-(1- piperazinyl)-1-ethanone | 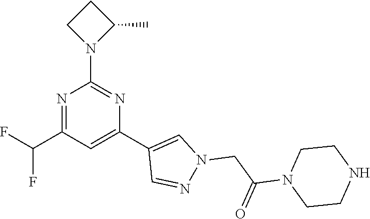 [SMILES: C[C@H]1CCN1c1nc(-c2cnn(CC(=O)N3CCNCC3)c2)cc(C(F)F)n1] |
| A8 | (2S,3R)-1-{5-methoxy-4-[1-(1- methyl-3-azetidinyl)-4-pyrazolyl]- 6-(trifluoromethyl)-2- pyrimidinyl}-2-methyl-3- azetidinol | 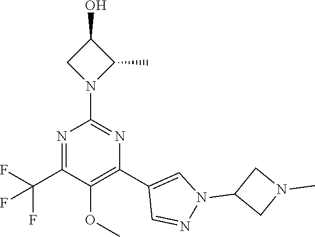 [SMILES: COc1c(-c2cnn(C3CN(C)C3)c2)nc(N2C[C@@H](O)[C@@H]2C)nc1C(F)(F)F] |
| A9 | (2S,3R)-1-{6-(difluoromethyl)-4- [1-(1-methyl-3-azetidinyl)-4- pyrazolyl]-2-pyrimidinyl}-2- methyl-3-azetidinol | 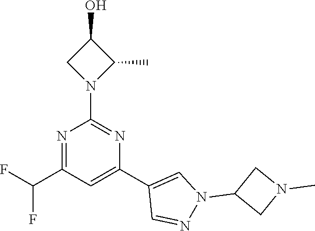 [SMILES: C[C@H]1[C@H](O)CN1c1nc(-c2cnn(C3CN(C)C3)c2)cc(C(F)F)n1] |
| A10 | (2S,3R)-1-{5-chloro-4-[1-(1- methyl-3-azetidinyl)-4-pyrazolyl]- 6-(trifluoromethyl)-2- pyrimidinyl}-2-methyl-3- azetidinol | 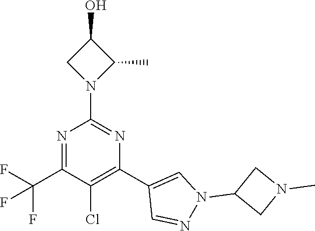 [SMILES: C[C@H]1[C@H](O)CN1c1nc(-c2cnn(C3CN(C)C3)c2)c(Cl)c(C(F)(F)F)n1] |
| A11 | (2S,3R)-1-{5-ethyl-4-[1-(1- methyl-3-azetidinyl)-4-pyrazolyl]- 6-(trifluoromethyl)-2- pyrimidinyl}-2-methyl-3- azetidinol | 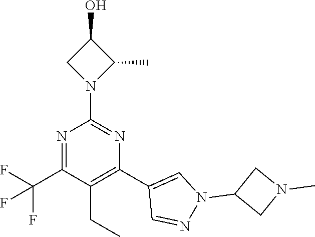 [SMILES: CCc1c(-c2cnn(C3CN(C)C3)c2)nc(N2C[C@@H](O)[C@@H]2C)nc1C(F)(F)F] |
| A12 | (2S,3R)-2-methyl-1-{5-methyl-4- [1-(1-methyl-3-azetidinyl)-4- pyrazolyl]-6-(trifluoromethyl)-2- pyrimidinyl}-3-azetidinol | 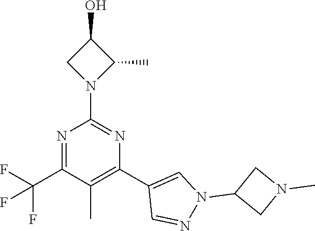 [SMILES: Cc1c(-c2cnn(C3CN(C)C3)c2)nc(N2C[C@@H](O)[C@@H]2C)nc1C(F)(F)F] |
| A13 | 2-(4-{2-[(R)-2-(difluoromethyl)-1- azetidinyl]-5-cyclopropyl-6- (difluoromethyl)-4-pyrimidinyl}- 1-pyrazolyl)-1-(1-piperazinyl)-1- ethanone | 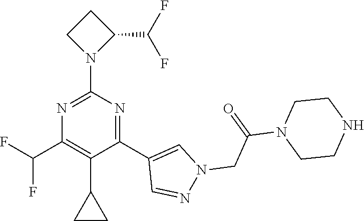 [SMILES: O=C(Cn1cc(-c2nc(N3CC[C@@H]3C(F)F)nc(C(F)F)c2C2CC2)cn1)N1CCNCC1] |
| A14 | 2-(4-{2-[(R)-2-(difluoromethyl)-1- azetidinyl]-6-(difluoromethyl)-4- pyrimidinyl}-1-pyrazolyl)-1-(1- piperazinyl)-1-ethanone | 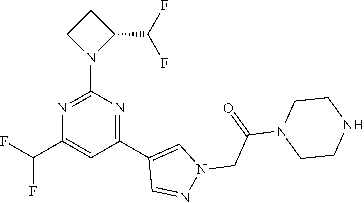 [SMILES: O=C(Cn1cc(-c2cc(C(F)F)nc(N3CC[C@@H]3C(F)F)n2)cn1)N1CCNCC1] |
| A15 | 2-(4-{2-[(R)-2-(trifluoromethyl)-1- azetidinyl]-6-(difluoromethyl)-5- methoxy-4-pyrimidinyl}-1- pyrazolyl)-1-(1-piperazinyl)-1- ethanone | 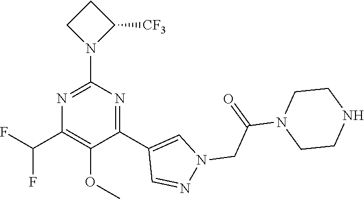 [SMILES: COc1c(-c2cnn(CC(=O)N3CCNCC3)c2)nc(N2CC[C@@H]2C(F)(F)F)nc1C(F)F] |
| A16 | 2-(4-{2-[(R)-2-(difluoromethyl)-1- azetidinyl]-5-fluoro-6- (trifluoromethyl)-4-pyrimidinyl}- 1-pyrazolyl)-1-(1-piperazinyl)-1- ethanone | 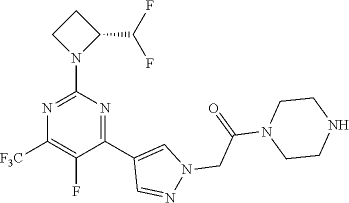 [SMILES: O=C(Cn1cc(-c2nc(N3CC[C@@H]3C(F)F)nc(C(F)(F)F)c2F)cn1)N1CCNCC1] |
| A17 | 2-(4-{2-[(R)-2-(difluoromethyl)-1- azetidinyl]-5-cyclopropyl-6- (trifluoromethyl)-4-pyrimidinyl}- 1-pyrazolyl)-1-(1-piperazinyl)-1- ethanone | 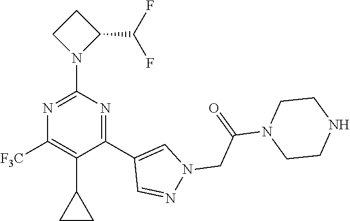 [SMILES: O=C(Cn1cc(-c2nc(N3CC[C@@H]3C(F)F)nc(C(F)(F)F)c2C2CC2)cn1)N1CCNCC1] |
| A18 | 2-(4-{2-[(R)-2-(trifluoromethyl)-1- azetidinyl]-6-(difluoromethyl)-5- methyl-4-pyrimidinyl}-1- pyrazolyl)-1-(1-piperazinyl)-1- ethanone | 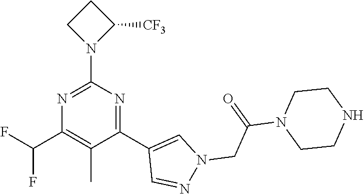 [SMILES: Cc1c(-c2cnn(CC(=O)N3CCNCC3)c2)nc(N2CC[C@@H]2C(F)(F)F)nc1C(F)F] |
| A19 | (2S,3R)-1-{6-(difluoromethyl)-5- methoxy-4-[1-(1-methyl-3- azetidinyl)-4-pyrazolyl]-2- pyrimidinyl}-2-methyl-3- azetidinol | 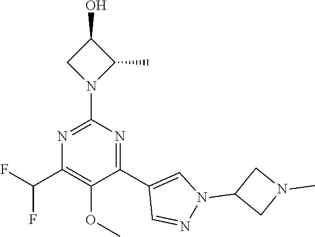 [SMILES: COc1c(-c2cnn(C3CN(C)C3)c2)nc(N2C[C@@H](O)[C@@H]2C)nc1C(F)F] |
| A20 | 2-(4-{2-[(R)-2-(trifluoromethyl)-1- azetidinyl]-6-(difluoromethyl)-4- pyrimidinyl}-1-pyrazolyl)-1-(1- piperazinyl)-1-ethanone | 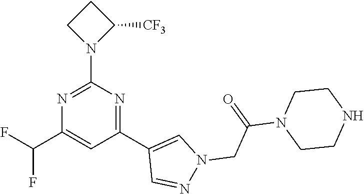 [SMILES: O=C(Cn1cc(-c2cc(C(F)F)nc(N3CC[C@@H]3C(F)(F)F)n2)cn1)N1CCNCC1] |
| A21 | (2S,3R)-1-{6-(difluoromethyl)-5- methyl-4-[1-(1-methyl-3- azetidinyl)-4-pyrazolyl]-2- pyrimidinyl}-2-methyl-3- azetidinol | 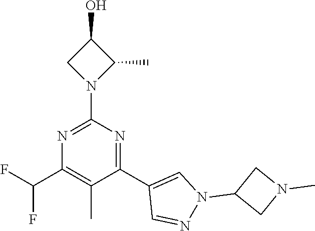 [SMILES: Cc1c(-c2cnn(C3CN(C)C3)c2)nc(N2C[C@@H](O)[C@@H]2C)nc1C(F)F] |
| A22 | 2-(4-{2-[(S)-2-methyl-1- azetidinyl]-6-(difluoromethyl)-5- fluoro-4-pyrimidinyl}-1- pyrazolyl)-1-(1-piperazinyl)-1- ethanone | 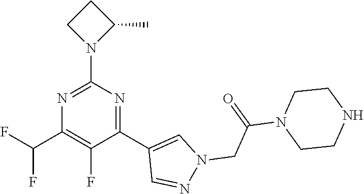 [SMILES: C[C@H]1CCN1c1nc(-c2cnn(CC(=O)N3CCNCC3)c2)c(F)c(C(F)F)n1] |
| A23 | 2-(4-{2-[(S)-2-methyl-1- azetidinyl]-6-(difluoromethyl)-5- methoxy-4-pyrimidinyl}-1- pyrazolyl)-1-(1-piperazinyl)-1- ethanone | 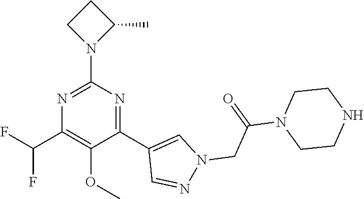 [SMILES: COc1c(-c2cnn(CC(=O)N3CCNCC3)c2)nc(N2CC[C@@H]2C)nc1C(F)F] |
| A24 | 2-(4-{2-[(S)-2-methyl-1- azetidinyl]-6-(difluoromethyl)-5- methyl-4-pyrimidinyl}-1- pyrazolyl)-1-(1-piperazinyl)-1- ethanone | 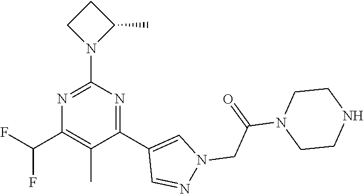 [SMILES: Cc1c(-c2cnn(CC(=O)N3CCNCC3)c2)nc(N2CC[C@@H]2C)nc1C(F)F] |
| A25 | (2R,3R)-1-{4-[1-(1-methyl-3- azetidinyl)-4-pyrazolyl]-6- (trifluoromethyl)-2-pyrimidinyl}- 2-(trifluoromethyl)-3-azetidinol | 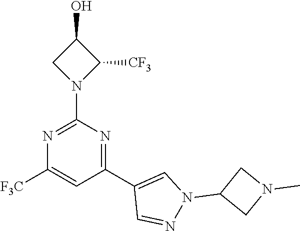 [SMILES: CN1CC(n2cc(-c3cc(C(F)(F)F)nc(N4C[C@@H](O)[C@@H]4C(F)(F)F)n3)cn2)C1] |
| A26 | 2-(4-{2-[(S)-2-methyl-1- azetidinyl]-6-(difluoromethyl)-5- ethyl-4-pyrimidinyl}-1-pyrazolyl)- 1-(1-piperazinyl)-1-ethanone | 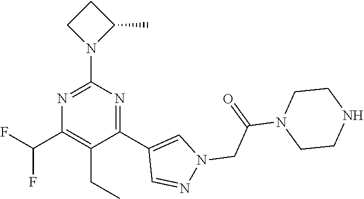 [SMILES: CCc1c(-c2cnn(CC(=O)N3CCNCC3)c2)nc(N2CC[C@@H]2C)nc1C(F)F] |
| A27 | (2S,3R)-1-{5-fluoro-4-[1-(1- methyl-3-azetidinyl)-4-pyrazolyl]- 6-(trifluoromethyl)-2- pyrimidinyl}-2-methyl-3- azetidinol | 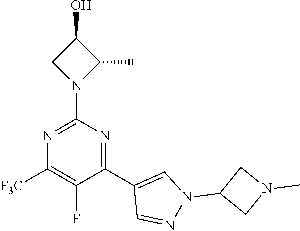 [SMILES: C[C@H]1[C@H](O)CN1c1nc(-c2cnn(C3CN(C)C3)c2)c(F)c(C(F)(F)F)n1] |
| A28 | 2-(4-{2-[(R)-2-(trifluoromethyl)-1- azetidinyl]-5-methoxy-6- (trifluoromethyl)-4-pyrimidinyl}- 1-pyrazolyl)-1-(1-piperazinyl)-1- ethanone | 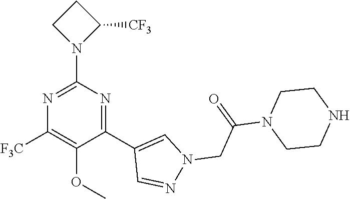 [SMILES: COc1c(-c2cnn(CC(=O)N3CCNCC3)c2)nc(N2CC[C@@H]2C(F)(F)F)nc1C(F)(F)F] |
| A29 | 2-(4-{2-[(R)-2-(trifluoromethyl)-1- azetidinyl]-5-chloro-6- (trifluoromethyl)-4-pyrimidinyl}- 1-pyrazolyl)-1-(1-piperazinyl)-1- ethanone | 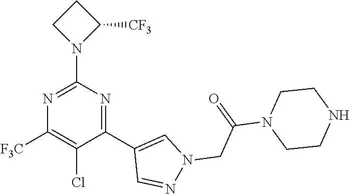 [SMILES: O=C(Cn1cc(-c2nc(N3CC[C@@H]3C(F)(F)F)nc(C(F)(F)F)c2Cl)cn1)N1CCNCC1] |
| A30 | 2-(4-{2-[(S)-2-methyl-1- azetidinyl]-5-cyclopropyl-6- (trifluoromethyl)-4-pyrimidinyl}- 1-pyrazolyl)-1-(1-piperazinyl)-1- ethanone | 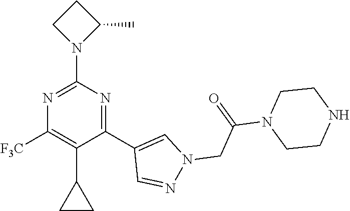 [SMILES: C[C@H]1CCN1c1nc(-c2cnn(CC(=O)N3CCNCC3)c2)c(C2CC2)c(C(F)(F)F)n1] |
| A31 | (2R,3R)-1-{4-[1-(3-azetidinyl)-4- pyrazolyl]-6-(trifluoromethyl)-2- pyrimidinyl}-2-(trifluoromethyl)- 3-azetidinol | 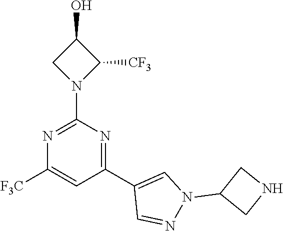 [SMILES: O[C@@H]1CN(c2nc(-c3cnn(C4CNC4)c3)cc(C(F)(F)F)n2)[C@H]1C(F)(F)F] |
| A32 | 2-(4-{2-[(S)-2-methyl-1- azetidinyl]-5-fluoro-6- (trifluoromethyl)-4-pyrimidinyl}- 1-pyrazolyl)-1-(1-piperazinyl)-1- ethanone | 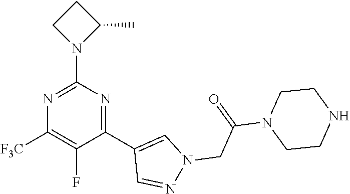 [SMILES: C[C@H]1CCN1c1nc(-c2cnn(CC(=O)N3CCNCC3)c2)c(F)c(C(F)(F)F)n1] |
| A33 | 2-(4-{2-[(R)-2-(difluoromethyl)-1- azetidinyl]-6-(trifluoromethyl)-4- pyrimidinyl}-1-pyrazolyl)-1-(1- piperazinyl)-1-ethanone | 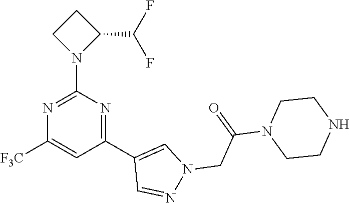 [SMILES: O=C(Cn1cc(-c2cc(C(F)(F)F)nc(N3CC[C@@H]3C(F)F)n2)cn1)N1CCNCC1] |
| A34 | (2S,3R)-1-{5-cyclopropyl-4-[1-(1- methyl-3-azetidinyl)-4-pyrazolyl]- 6-(trifluoromethyl)-2- pyrimidinyl}-2-methyl-3- azetidinol | 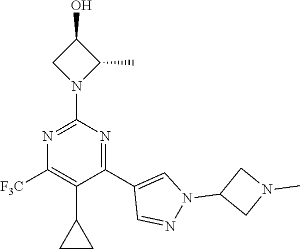 [SMILES: C[C@H]1[C@H](O)CN1c1nc(-c2cnn(C3CN(C)C3)c2)c(C2CC2)c(C(F)(F)F)n1] |
| A35 | (2S,3R)-1-{4-[1-(3-azetidinyl)-4- pyrazolyl]-6-(difluoromethyl)-5- methyl-2-pyrimidinyl}-2-methyl- 3-azetidinol | 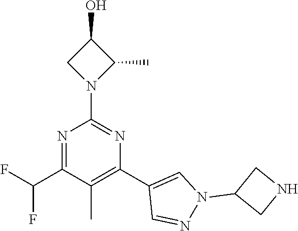 [SMILES: Cc1c(-c2cnn(C3CNC3)c2)nc(N2C[C@@H](O)[C@@H]2C)nc1C(F)F] |
| A36 | 2-(4-{2-[(R)-2-(difluoromethyl)-1- azetidinyl]-5-methoxy-6- (trifluoromethyl)-4-pyrimidinyl}- 1-pyrazolyl)-1-(1-piperazinyl)-1- ethanone | 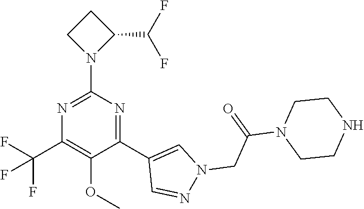 [SMILES: COc1c(-c2cnn(CC(=O)N3CCNCC3)c2)nc(N2CC[C@@H]2C(F)F)nc1C(F)(F)F] |
| A37 | 2-(4-{2-[(R)-2-(difluoromethyl)-1- azetidinyl]-5-chloro-6- (difluoromethyl)-4-pyrimidinyl}- 1-pyrazolyl)-1-(1-piperazinyl)-1- ethanone | 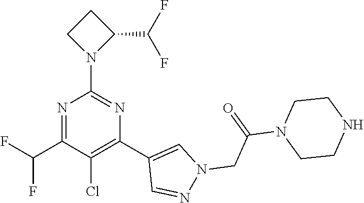 [SMILES: O=C(Cn1cc(-c2nc(N3CC[C@@H]3C(F)F)nc(C(F)F)c2Cl)cn1)N1CCNCC1] |
| A38 | 2-(4-{2-[(R)-2-(difluoromethyl)-1- azetidinyl]-5-chloro-6- (trifluoromethyl)-4-pyrimidinyl}- 1-pyrazolyl)-1-(1-piperazinyl)-1- ethanone | 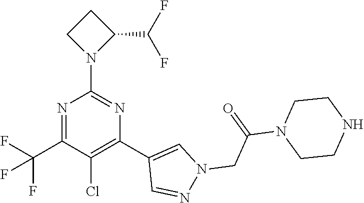 [SMILES: O=C(Cn1cc(-c2nc(N3CC[C@@H]3C(F)F)nc(C(F)(F)F)c2Cl)cn1)N1CCNCC1] |
| A39 | 2-(4-{2-[(R)-2-(difluoromethyl)-1- azetidinyl]-6-(difluoromethyl)-5- fluoro-4-pyrimidinyl}-1- pyrazolyl)-1-(1-piperazinyl)-1- ethanone | 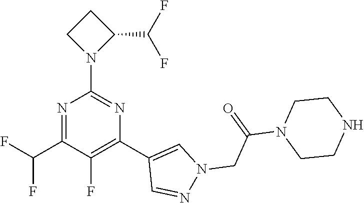 [SMILES: O=C(Cn1cc(-c2nc(N3CC[C@@H]3C(F)F)nc(C(F)F)c2F)cn1)N1CCNCC1] |
| A40 | 2-(4-{2-[(R)-2-(difluoromethyl)-1- azetidinyl]-6-(difluoromethyl)-5- ethyl-4-pyrimidinyl}-1-pyrazolyl)- 1-(1-piperazinyl)-1-ethanone | 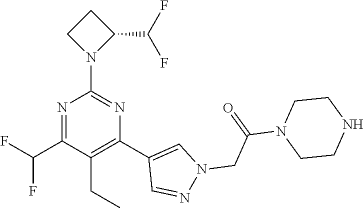 [SMILES: CCc1c(-c2cnn(CC(=O)N3CCNCC3)c2)nc(N2CC[C@@H]2C(F)F)nc1C(F)F] |
| A41 | 2-(4-{2-[(R)-2-(difluoromethyl)-1- azetidinyl]-5-ethyl-6- (trifluoromethyl)-4-pyrimidinyl}- 1-pyrazolyl)-1-(1-piperazinyl)-1- ethanone | 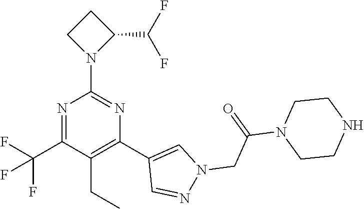 [SMILES: CCc1c(-c2cnn(CC(=O)N3CCNCC3)c2)nc(N2CC[C@@H]2C(F)F)nc1C(F)(F)F] |
| A42 | 2-(4-{2-[(R)-2-(difluoromethyl)-1- azetidinyl]-6-(difluoromethyl)-5- methyl-4-pyrimidinyl}-1- pyrazolyl)-1-(1-piperazinyl)-1- ethanone | 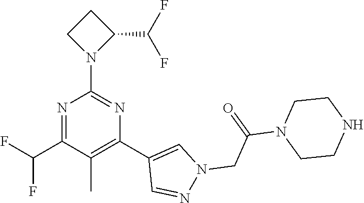 [SMILES: Cc1c(-c2cnn(CC(=O)N3CCNCC3)c2)nc(N2CC[C@@H]2C(F)F)nc1C(F)F] |
| A43 | 2-(4-{2-[(R)-2-(difluoromethyl)-1- azetidinyl]-5-methyl-6- (trifluoromethyl)-4-pyrimidinyl}- 1-pyrazolyl)-1-(1-piperazinyl)-1- ethanone | 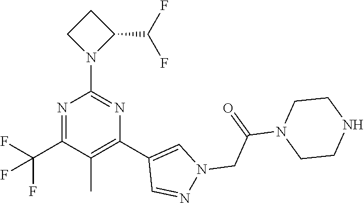 [SMILES: Cc1c(-c2cnn(CC(=O)N3CCNCC3)c2)nc(N2CC[C@@H]2C(F)F)nc1C(F)(F)F] |
| A44 | 2-(4-{2-[(R)-2-(difluoromethyl)-1- azetidinyl]-6-(difluoromethyl)-5- methoxy-4-pyrimidinyl}-1- pyrazolyl)-1-(1-piperazinyl)-1- ethanone | 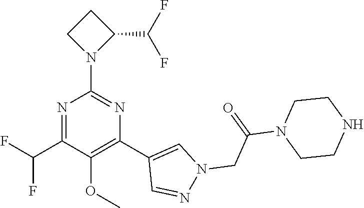 [SMILES: COc1c(-c2cnn(CC(=O)N3CCNCC3)c2)nc(N2CC[C@@H]2C(F)F)nc1C(F)F] |

In some case, the compound is selected from Compound A3, A9, A12, A19, A21, A24, A35, and pharmaceutically acceptable salts thereof. In some cases, the compound is selected from Compound A3, A24, and pharmaceutically acceptable salts thereof. In some cases, the compound is selected from Compound A21, A35, and pharmaceutically acceptable salts thereof. In some cases, the compound is selected from Compound A9, A12, A19, and pharmaceutically acceptable salts thereof.

### Abbreviations

LC/MS=Liquid Chromatography Mass Spectrometry
TFA=Trifluoroacetic acid
min=minutes
mL=milliliters
FA=Formic acid
DTBPF=1,1′-Bis(di-tert-butylphosphino)ferrocene
Oxone=Tetrabutylammonium hydrogen monopersulfate
DMF=Dimethylformamide
DBU=1,8-Diazabicyclo[5.4.0]undec-7-ene
BOP=Benzotriazole-1-yl-oxy-tris-(dimethylamino)-phosphonium hexafluorophosphate
Ts=4-Toluenesulfonate
ACN and MeCN=Acetonitrile
DIEA=N,N-Diisopropylethylamine
DCM=Dichloromethane
TLC=Thin-layer Chromatography
HATU=1-[Bis(dimethylamino)methylene]-1H-1,2,3-triazolo[4,5-b]pyridinium 3-oxid hexafluorophosphate
NMR=Nuclear Magnetic Resonance
NMP=1-Methyl-2-pyrrolidone
RT=Retention Time
TEA=Triethylamine
THF=tetrahydrofuran
TBDMS=tert-butyldimethylsilyl
aq=aqueous
eq=equivalents
dppf=1,1′-Ferrocenediyl-bis(diphenylphosphine)
DAST=(Diethylamino)sulfur trifluoride
FCC=Flash Column Chromatography
HPLC=High-performance liquid chromatography
Bpin=4,4,5,5-tetramethyl-1,3,2-dioxaborolan-2-yl
Tf=trifluoromethylsulfonyl
PMB=4-Methoxybenzyl
Bn=Benzyl
LAH=Lithium aluminum hydride

### General Synthetic Methods

Certain processes for the manufacture of the compounds of this disclosure are provided as further features of the disclosure and are illustrated by the following exemplary reaction schemes. Those skilled in the art will appreciate that other synthetic routes may be used to synthesize the inventive compounds. For a more detailed description of the individual reaction steps; see the Examples section herein. Although specific starting materials and reagents are depicted in the schemes and discussed herein, other starting materials and reagents can be easily substituted to provide a variety of derivatives and/or reaction conditions. In addition, many of the compounds prepared by the methods described herein can be further modified in light of this disclosure using conventional chemistry well known to those skilled in the art. In particular, it is noted that the compounds prepared according to these Schemes may be modified further to provide new Examples within the scope of this disclosure. In addition, it will be evident from the detailed descriptions given in the Experimental section that the modes of preparation employed extend further than the general procedures described herein. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8^{th} Ed., Ed.: Smith, M. B., John Wiley & Sons, New York: 2019, the entire contents of which are hereby incorporated by reference.

The starting materials are generally available from commercial sources such as Merck Sigma-Aldrich Inc. and Enamine Ltd. Aldrich Chemicals (Milwaukee, Wis.) or are readily prepared using methods known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including Supplements (also available via the Beilstein online database).

As an initial note, in the preparation of compounds of the present disclosure, it is noted that some of the preparation methods useful for the preparation of the compounds described herein may require protection of remote functionality (e.g., primary amine, secondary amine, carboxyl in intermediates). The need for such protection will vary depending on the nature of the remote functionality and the conditions of the preparative methods and can be readily determined by one of ordinary skill in the art. The use of such protection/deprotection methods is also within the ordinary skill in the art. For a general description of protecting groups and their use, see T. W. Greene, Protective Groups in Organic Synthesis, John Wiley & Sons, New York, 1991 and Greene's Protective Groups in Organic Synthesis, John Wiley & Sons, New York 2006.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the Formula I compound.

Compounds of Formula I, or salts thereof, may be prepared by a variety of methods known to those skilled in the art Nonlimiting examples of these methods are outlined in the following schemes, preparations and examples. All substituents are as defined herein, unless indicated otherwise. Reagents, solvents and starting materials are commercially available, known in the literature or readily accessible to one skilled in the art. Products of each synthetic procedure can be recovered and isolated by conventional methods well known in the art, including extraction, evaporation, precipitation, chromatography, filtration, trituration and crystallization.

Compounds of Formula I may be isolated as racemates, enantiomers or diastereoisomers using well-known techniques such as crystallization, chiral chromatography, or supercritical fluid chromatography. These techniques may be applied at the appropriate point in the synthesis. The Formula I compound diastereomers/enantiomers can be prepared as racemic mixtures followed by appropriate chiral separation as described herein or by for example reaction with the desired chiral substituted azetidine compound.

One skilled in the art will appreciate that compounds of Formula I, or salts thereof, can also be synthesized following similar methods to those described herein, with modifications, for example: a) the use of appropriate protection/deprotection strategies; b) the use of the appropriately substituted starting materials and reactants; c) appropriate changes to the order of synthetic steps; d) transformation of one functional group to another; e) use of an alternative stereochemical configuration.

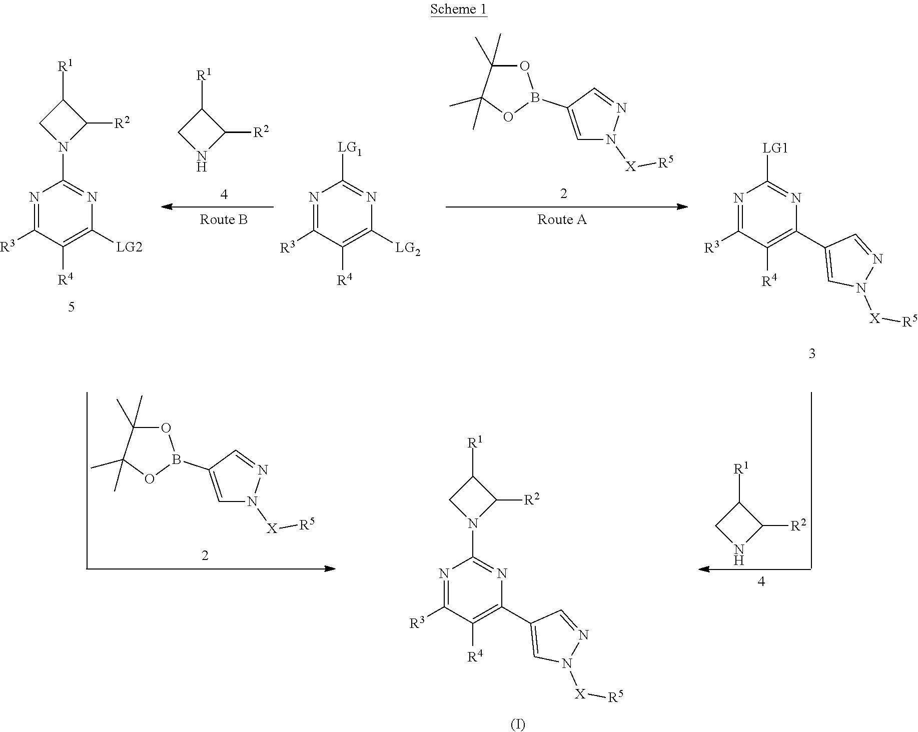

Scheme 1 provides a general strategy for the preparation of compounds of Formula I. LG is defined as a “leaving group”, utilized in certain reactions for the synthesis of compounds of Formula I. Route A shows the conversion of pyrimidine 1 to pyrazole compound 3 by Suzuki coupling between boronate 2 and LG2 of 1. Nucleophilic aromatic substitution (SN_{Ar}) reaction of compound 3 (LG1) with azetidine 4 provides compounds of Formula I. Route B shows an alternative strategy to convert pyrimidine 1 to azetidine compound 5 by SN_{Ar} reaction between LG1 of 1 and azetidine 4. Subsequent Suzuki coupling of boronate 2 with 5 (LG2) provides compounds of Formula I. The synthesis of compounds following these strategies utilises appropriately substituted reactants, appropriate protection/deprotection steps, and other functional group transformations, as necessary.

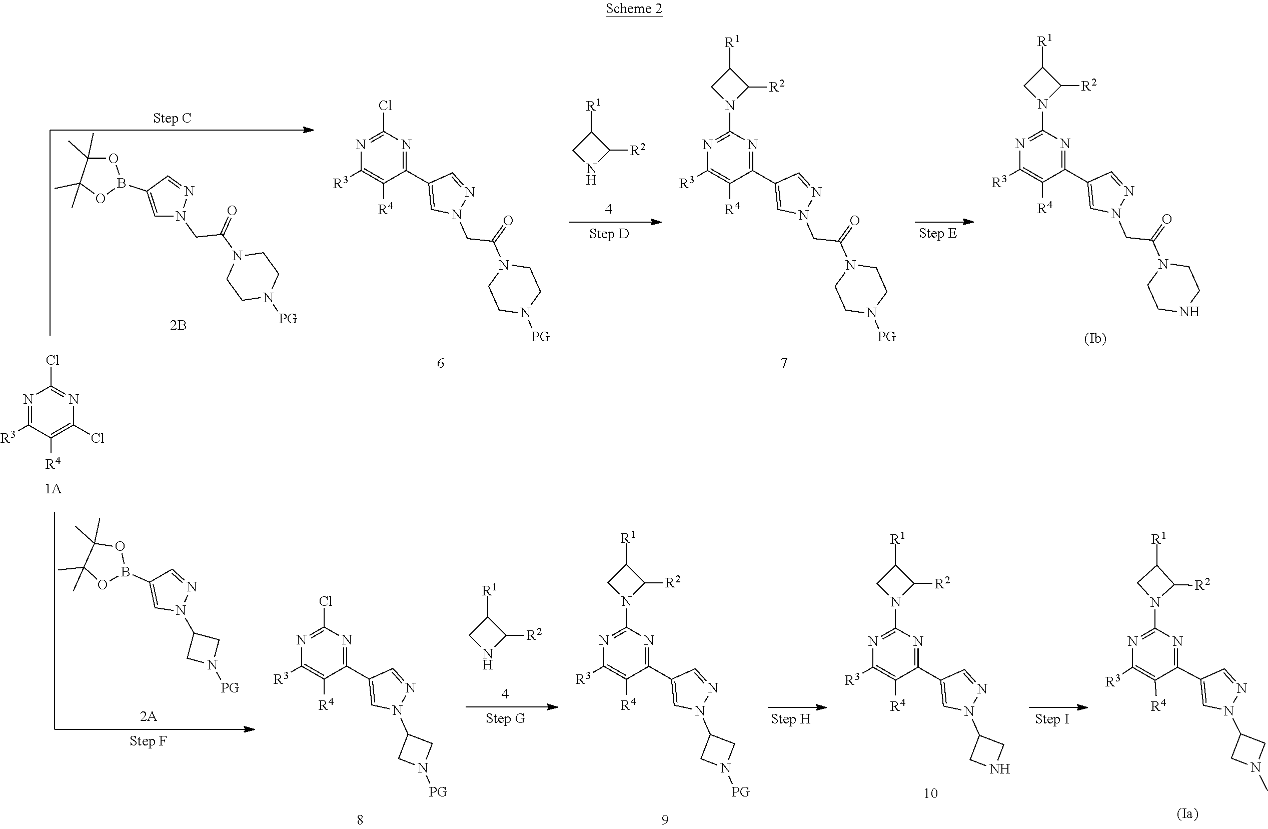

Scheme n shows one strategy to synthesize a subset of compounds of Formula I, namely compounds of Formula Ia and Ib. PG is defined as a nitrogen protecting group, for example BOC, which can be removed an appropriate moment during the synthesis or at the end.

In Step C, dichloro pyrimidine 1A undergoes a Suzuki reaction with boronate 2B to give pyrazole compound 6. This reaction is performed with a base, for example Na₂CO₃, in an organic solvent, for example 1,4-dioxane, in the presence of a palladium catalyst, such as tetrakis(triphenylphosphine)palladium(0), at elevated temperature. Compound 6 then undergoes an SN_{Ar} reaction in Step D with azetidine 4 to give compound 7. Typical reaction conditions include a base, for example K₂CO₃, in an organic solvent such as NMP at elevated temperature. In Step E compound 7 is deprotected, for example using TFA to remove a BOC group, to yield compounds of Formula (1b).

Compound 1A also undergoes a Suzuki reaction with boronate 2A in Step F to afford pyrazole 6. Reaction conditions are similar to those for Step C, using DTBPF PdCl₂ as the palladium catalyst. Compound 8 then undergoes an SN_{M} reaction in Step G with azetidine 4 to give compound 9. Typical reaction conditions include abase, for example triethylamine, in an organic solvent such as THF, at elevated temperature. In Step H, compound 9 is deprotected, for example using TFA to remove a BOC group, to give compound 10, which is then methylated in Step I to yield compounds of Formula (1a). Typical reaction conditions include the use of formaldehyde and a reducing agent, for example NaBH(OAc)₃, in an organic solvent such as dichloromethane at room temperature.

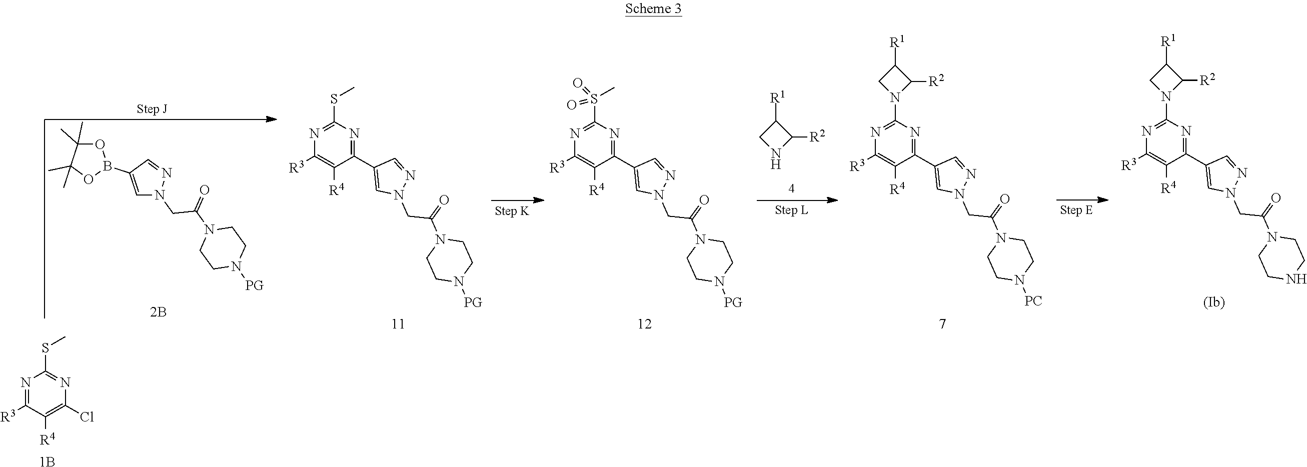

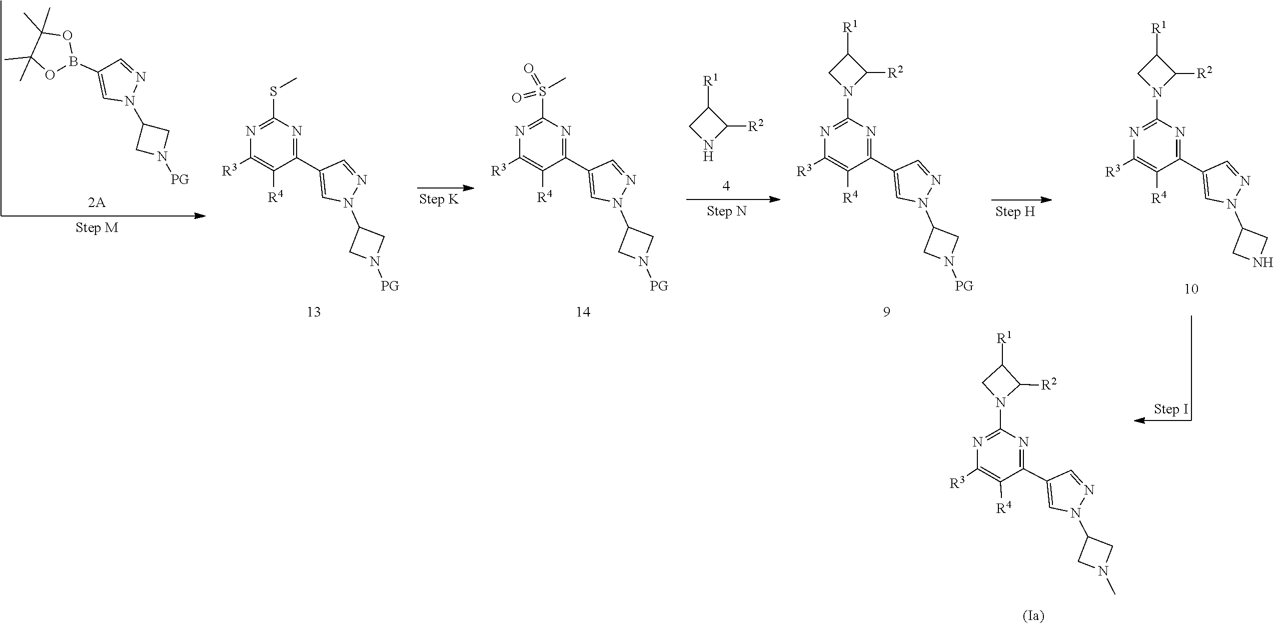

Scheme 3 shows another strategy to synthesize a subset of compounds of Formula I, namely compounds of Formula Ia and Ib. PG is defined as a nitrogen protecting group, for example BOC, which can be removed at an appropriate moment during the synthesis or at the end.

In Step J, using similar conditions to Step C from Scheme 2, chloro pyrimidine 1B undergoes a Suzuki reaction with boronate 2B to give pyrazole compound 11. Compound 11 is then oxidized in Step K to give sulfone 12. Typical reaction conditions include the use of an oxidizing agent, for example Oxone, in an organic solvent such as dimethylformamide at room temperature. In Step L, compound 12 undergoes an SN_{Ar} reaction with azetidine 4 to give compound 7. Typical reaction conditions include a base, for example triethylamine, in an organic solvent such as NMP, at elevated temperature. Compound 7 is deprotected, as described in Step E, Scheme 2, to provide compounds of Formula (1b).

Compound 1B also undergoes a Suzuki reaction with boronate 2A in Step M to afford pyrazole 13. Reaction conditions are similar to those for Step C from Scheme 2, using DTBPF PdCl₂ as the palladium catalyst. Compound 13 is then oxidized in Step K (described above) to give sulfone 14. In Step N, using similar conditions to Step L, compound 14 then undergoes an SN_{Ar} reaction with azetidine 4 to give compound 9, which is converted to compounds of Formula (1a), as describe in Scheme 2.

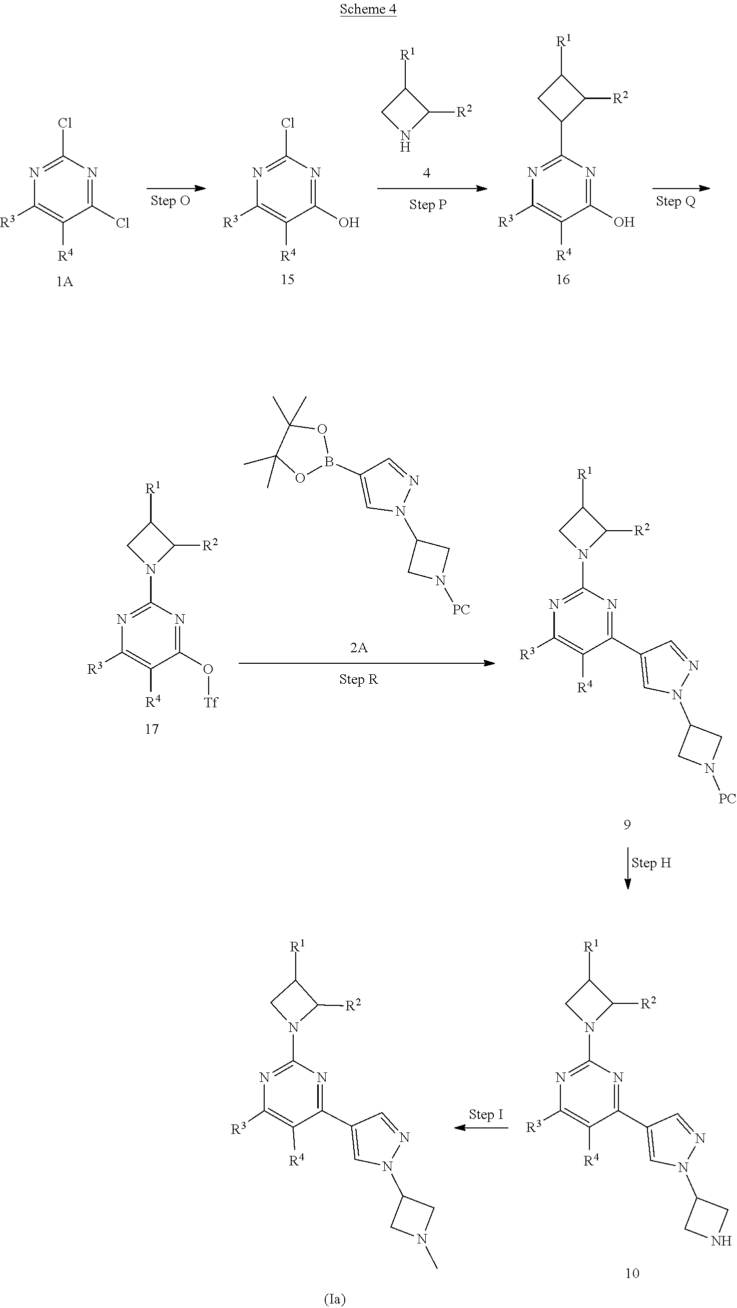

Scheme 4 shows a further strategy to synthesize a subset of compounds of Formula I, namely compounds of Formula Ia. PG is defined as a nitrogen protecting group, for example BOC, which can be removed at an appropriate moment during the synthesis or at the end. Tf is defined as trifluoromethyl sulfonate, which provides —OTf as a reactive group that can be used in Suzuki couplings.

In Step 0, dichloro pyrimidine 1A is hydrolyzed using sodium hydroxide in aqueous THF at elevated temperature to give compound 15. In Step P compound 15 undergoes an SN_{Ar} reaction with azetidine 4 to give compound 16. Typical reaction conditions include a base, for example diisopropylethylamine, in an organic solvent such as acetonitrile, at elevated temperature in a microwave. Compound 16 is then converted to triflate 17 in Step Q, using N-phenyltrifluoromethanesulformamide in the presence of a base, for example diisopropylethylamine, in an organic solvent such as dimethylformamide at room temperature. In Step R, triflate 17 undergoes a Suzuki reaction with boronate 2A to afford compound 9. This reaction is performed with a base, for example K₃PO₄, in an organic solvent, for example 1,4-dioxane, in the presence of a palladium catalyst, such as Pd(dppf)Cl₂, at elevated temperature. Compound 9 is then converted to compounds of Formula (1a), as describe in Scheme 2.

As can be appreciated by the skilled artisan, the foregoing synthetic Schemes and representative examples (herein) are not intended to comprise a comprehensive list of all means by which the compounds described and claimed in this application may be synthesized. Further methods will be evident to those of ordinary skill in the art. Additionally, the various synthetic steps described herein may be performed in an alternate sequence or order to give the desired compounds. The disclosure further encompasses “intermediate” compounds, including structures produced from the synthetic procedures described, whether isolated or generated in-situ and not isolated, prior to obtaining the finally desired compound. These intermediates are included in the scope of this disclosure. Exemplary embodiments of such intermediate compounds are set forth in the Examples herein.

### Pharmaceutically Acceptable Salts

The compounds described herein can exist in free form, or, where appropriate, as salts. Those salts that are pharmaceutically acceptable are of particular interest since they are useful in administering the compounds described herein for medical purposes. Salts that are not pharmaceutically acceptable are useful in manufacturing processes, for isolation and purification purposes, and in some instances, for use in separating stereoisomeric forms of the compounds of the disclosure or intermediates thereof.

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, pyroglutamate, salicylate, stearate, succinate, sulfate, tartrate, thiocyanate, p-toluenesulfonate, undecanoate, valerate salts, and the like.

Other acids and bases, while not in themselves pharmaceutically acceptable, may be employed in the preparation of salts useful as intermediates in obtaining the compounds described herein and their pharmaceutically acceptable acid or base addition salts.

It should be understood that a compound disclosed herein can be present as a mixture/combination of different pharmaceutically acceptable salts. Also contemplated are mixtures/combinations of compounds in free form and pharmaceutically acceptable salts.

### Prodrugs

The present disclosure also includes the prodrugs of compounds of Formula I (or any of the embodiments thereof described herein) and/or a pharmaceutically acceptable salt thereof. The term prodrug is intended to represent covalently bonded carriers, which are capable of releasing the active ingredient of Formula I (or any of the embodiments thereof described herein) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups, however, regenerate original functional groups in vivo or by routine manipulation. Prodrugs of compounds of Formula I (or any of the embodiments thereof described herein) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula I), amides (e.g., trifluoroacetylamino, acetylamino, and the like), and the like. Prodrugs of compounds of Formula I (or any of the embodiments thereof described herein) and/or a pharmaceutically acceptable salt thereof are also within the scope of this disclosure.

### Metabolites

Also included within the scope of the disclosure are metabolites of compounds of Formula I, that is, compounds formed in vivo upon administration of the drug. Some examples of metabolites in accordance with the disclosure include:
(i) where the compound of Formula I contains a methyl group, an hydroxymethyl derivative thereof (—CH₃->—CH₂OH);
(ii) where the compound of Formula I contains an alkoxy group, an hydroxy derivative thereof (—OR->—OH);
(iii) where the compound of Formula I contains a tertiary amino group, a secondary amino derivative thereof (—NRR->—NHR or —NRH);
(iv) where the compound of Formula I contains a secondary amino group, a primary derivative thereof (—NHR->—NH₂);
(v) where the compound of Formula I contains a phenyl moiety, a phenol derivative thereof (—Ph->—PhOH); and
(vi) where the compound of Formula I contains an amide group, a carboxylic acid derivative thereof (—CONH₂->COOH).

### Hydrates and Solvates

The compounds described herein include hydrates and solvates of the compounds or pharmaceutically acceptable salts thereof. The term solvate is used herein to describe a molecular complex comprising the compound of the disclosure and one or more pharmaceutically acceptable solvent molecules, for example, ethanol. Such solvent molecules are those commonly used in the pharmaceutical art, which are known to be innocuous to the recipient, e.g., water, ethanol, and the like. Other solvents may be used as intermediate solvates in the preparation of more desirable solvates, such as methanol, methyl t-butyl ether, ethyl acetate, methyl acetate, (S)-propylene glycol, (R)-propylene glycol, 1,4-butyne-diol, and the like.

The term hydrate is employed when said solvent is water. Pharmaceutically acceptable solvates include hydrates and other solvates wherein the solvent of crystallization may be isotopically substituted, e.g., D₂O. d-acetone, d-DMSO. The solvates and/or hydrates preferably exist in crystalline form. A classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

Also included within the scope of the disclosure are multi-component complexes (other than salts and solvates) wherein the drug and at least one other component are present in stoichiometric or non-stoichiometric amounts. The compounds of the disclosure may also exist as complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized. For a review of such complexes, see J Pharm Sci, 64 (8), 1269-1288 by Haleblian (August 1975).

The compounds of the disclosure may be combined with soluble macromolecular entities, such as cyclodextrin and suitable derivatives thereof or polyethylene glycol-containing polymers, in order to improve their pharmacokinetic profile, solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-infusion complexes may be used. As an alternative to direct complexation with the drug, the cyclodextrin may be used as an auxiliary additive, i.e. as a carrier, diluent, or solubilizer. Most commonly used for these purposes are alpha-, beta- and gamma-cyclodextrins.

### Polymorphs

The present disclosure also includes polymorphic forms (amorphous as well as crystalline).

The compounds of the disclosure may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

Certain compounds of the present disclosure or combination agents may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the present disclosure followed by gradual or fast cooling. The presence of polymorphs may be determined by solid probe NMR spectroscopy, IR spectroscopy, differential scanning calorimetry, powder X-ray diffraction or such other techniques.

### Pharmaceutical Compositions

The compounds described herein can be formulated into pharmaceutical compositions that further comprise a pharmaceutically acceptable carrier, diluent, adjuvant or vehicle. In embodiments, the present disclosure relates to a pharmaceutical composition comprising a compound described herein or salt thereof, and a pharmaceutically acceptable carrier, diluent, adjuvant or vehicle. In embodiments, the pharmaceutical composition comprises a safe and effective amount of a compound as disclosed herein or a pharmaceutically acceptable salt thereof and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

An “effective amount” includes a “therapeutically effective amount” and a “prophylactically effective amount”. The term “therapeutically effective amount” refers to an amount effective in treating and/or ameliorating diseases or disorders associated with KHK dysregulation in a patient. The term “prophylactically effective amount” refers to an amount effective in preventing and/or substantially lessening the chances of developing diseases or disorders associated with KHK dysregulation.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon their administration to a subject. Standard pharmaceutical formulation techniques can be employed.

The pharmaceutically acceptable carrier, adjuvant, or vehicle, as used herein, includes any solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, its use is contemplated to be within the scope of this disclosure. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky. Side effects include, but are not limited to fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

Some examples of materials which can serve as pharmaceutically acceptable carriers include, but are not limited to, ion exchangers, alumina, aluminum stearate, lecithin, serum proteins (such as human serum albumin), buffer substances (such as tween 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In some embodiments, the pharmaceutical compositions disclosed herein can be formulated with supplementary active ingredients.

The carrier can be a solvent or dispersion medium containing, for example, water, ethanol, polyol (for example, glycerol, propylene glycol and liquid polyethylene glycol, and the like), suitable mixtures thereof, and/or vegetable oils. The proper fluidity can be maintained, for example, by the use of a coating such as, for example, lecithin, by the maintenance of the required particle size in the case of dispersion and by the use of surfactants. Preventing the action of microorganisms in the compositions disclosed herein is achieved by adding antibacterial and/or antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, a pharmaceutical composition can be within a matrix which controls the release of the composition. In some embodiments, the matrix can comprise lipid, polyvinyl alcohol, polyvinyl acetate, polycaprolactone, poly(glycolic)acid, poly(lactic)acid, polycaprolactone, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SAIB), and combinations thereof and other polymers such as those disclosed, for example, in U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, each of which is expressly incorporated by reference herein in its entirety. In these embodiments, the matrix sustainedly releases the drug.

Pharmaceutically acceptable carriers and/or diluents may also include any solvents, dispersion media, coatings, antibacterials and/or antifungals, isotonic and absorption delaying agents and the like. The use of such media and agents for pharmaceutically active substances is well known in the art. Except insofar as any conventional medium or agent is incompatible with the active ingredient, use thereof in the pharmaceutical compositions is contemplated.

In some embodiments, the pharmaceutical composition is in the form of an aqueous suspension, which can be prepared from solutions or suspensions. With respect to solutions or suspensions, dosage forms can be comprised of micelles of lipophilic substances, liposomes (phospholipid vesicles/membranes) and/or a fatty acid (e.g., palmitic acid). In particular embodiments, the pharmaceutical composition is a solution or suspension that is capable of dissolving in the fluid secreted by mucous membranes of the epthelium of the tissue to which it is administered, applied and/or delivered, which can advantageously enhance absorption.

The pharmaceutical composition can be an aqueous solution, a nonaqueous solution or a combination of an aqueous and nonaqueous solution. Suitable aqueous solutions include, but are not limited to, aqueous gels, aqueous suspensions, aqueous microsphere suspensions, aqueous microsphere dispersions, aqueous liposomal dispersions, aqueous micelles of liposomes, aqueous microemulsions, and any combination of the foregoing, or any other aqueous solution that can dissolve in the fluid secreted by the mucosal membranes of the nasal cavity. Exemplary nonaqueous solutions include, but are not limited to, nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing, or any other nonaqueous solution that can dissolve or mix in the fluid secreted by mucosal membranes.

Examples of powder formulations include, without limitation, simple powder mixtures, micronized powders, freeze dried powder, lyophilized powder, powder microspheres, coated powder microspheres, liposomal dispersions, and any combination of the foregoing. Powder microspheres can be formed from various polysaccharides and celluloses, which include without limitation starch, methylcellulose, xanthan gum, carboxymethylcellulose, hydroxypropyl cellulose, carbomer, alginate polyvinyl alcohol, acacia, chitosans, and any combination thereof.

The pharmaceutical composition can also optionally include an absorption enhancer, such as an agent that inhibits enzyme activity, reduces mucous viscosity or elasticity, decreases mucociliary clearance effects, opens tight junctions, and/or solubilizes the active compound. Chemical enhancers are known in the art and include chelating agents (e.g., EDTA), fatty acids, bile acid salts, surfactants, and/or preservatives. Enhancers for penetration can be particularly useful when formulating compounds that exhibit poor membrane permeability, lack of lipophilicity, and/or are degraded by aminopeptidases. The concentration of the absorption enhancer in the pharmaceutical composition will vary depending upon the agent selected and the formulation.

To extend shelf life, preservatives can optionally be added to the pharmaceutical composition. Suitable preservatives include but are not limited to benzyl alcohol, parabens, thimerosal, chlorobutanol and benzalkonium chloride, and combinations of the foregoing. The concentration of the preservative will vary depending upon the preservative used, the compound being formulated, the formulation, and the like. In representative embodiments, the preservative is present in an amount of about 2% by weight or less.

As another option, the composition can comprise a flavoring agent, e.g., to enhance the taste and/or acceptability of the composition to the subject.

### Routes of Administration and Dosages

The compounds and pharmaceutically acceptable compositions described herein can be administered to humans and other animals orally, rectally, parenterally, intracistemally, intravaginally, intraperitoneally, topically (as by powders, ointments, or drops) or bucally. In some embodiments, the compound or composition disclosed herein is administered orally, via inhalation, or intravenously.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsulated matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are specifically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include buccal films, capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite day, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. In the case of buccal films, a film can employ a water-dissolving polymer, which allows the film to quickly hydrate, adhere, and dissolve when placed on the tongue, or in the oral cavity, which results in systemic drug delivery.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in microencapsulated form with one or more excipients as noted herein. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions described herein may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions described herein may be administered in the form of suppositories for rectal administration. These can be prepared by mixing the agent with a suitable non-irritating excipient which is solid at room temperature but liquid at rectal temperature and therefore will melt in the rectum to release the drug. Such materials include, but are not limited to, cocoa butter, beeswax and polyethylene glycols.

Topical application for the lower intestinal tract can be effected in a rectal suppository formulation (as described herein) or in a suitable enema formulation. Topical application also includes the use of transdermal patches.

For topical applications, the pharmaceutical compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH adjusted sterile saline, or, specifically, as solutions in isotonic, pH adjusted sterile saline, either with or without a preservative such as benzalkonium chloride. Alternatively, for ophthalmic uses, the pharmaceutical compositions may be formulated in an ointment such as petrolatum.

The pharmaceutical compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds for use in the methods of the disclosure can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

### Methods of Treatment

Provided herein are uses of a compound described herein as a therapeutic agent. The compounds described herein or pharmaceutically acceptable salts thereof can be used to inhibit ketohexokinase (KHK), and to treat or prevent diseases or disorders (e.g., wherein the diseases or disorders are associated with KHK dysregulation or fructose metabolism, including secondary to excessive consumption of fructose and/or alcohol) in a biological sample (e.g., a cell culture) or in humans (e.g., in a subject). The compounds described herein or pharmaceutically acceptable salts thereof can be used in methods of treating or preventing diseases or disorders associated with KHK dysregulation or fructose metabolism, including secondary to excessive consumption of fructose and/or alcohol. The compounds, pharmaceutical compositions, and methods of the present disclosure can be useful for treating a subject such as, but not limited to, a mammal, a human, a non-human mammal, a domesticated animal (e.g., laboratory animals, household pets, or livestock), a non-domesticated animal (e.g., wildlife), a dog, a cat, a rodent, a mouse, a hamster, a cow, a bird, a chicken, a fish, a pig, a horse, a goat, a sheep, or a rabbit, preferably a human. Thus, the compounds can be used to treat a disease, disorder, condition, or associated co-morbidity (referred to generally herein as a disease) selected from any one or more of the following: type 1 diabetes mellitus (T1D), type 2 diabetes mellitus (T2D), idiopathic T1D, latent autoimmune diabetes of adults (LADA), early-onset diabetes (EOD), atypical diabetes, maturity-onset diabetes of the young (MODY), malnutrition-related diabetes, gestational diabetes, hyper-glycemia, insulin resistance, hepatic insulin resistance, impaired glucose tolerance, diabetic neuropathy, diabetic nephropathy, diabetic kidney disease (DKD), kidney disease, acute kidney disorder, tubular dysfunction, proinflammatory changes to the proximal tubules, diabetic retinopathy, adipocyte dysfunction, visceral adipose deposition, obesity, eating disorders, excessive sugar craving, excessive alcohol consumption, dyslipidemia, hyperlipidemia, hypertriglyceridemia, increased total cholesterol, high LDL cholesterol, high non HDL cholesterol, low HDL cholesterol, hyperinsulinemia, nonalcoholic fatty liver disease (NAFLD), metabolic dysfunction-associated steatotic liver disease (MASLD), non-alcoholic steatohepatitis (NASH), metabolic dysfunction-associated steatohepatitis (MASH), MASLD with increased alcohol intake (MetALD), liver steatosis, fibrosis, cirrhosis, hepatocellular carcinoma, hereditary fructose intolerance (HFI), alcoholic steatohepatitis (ASH), viral liver disease, diseases associated with liver fibrosis or cirrhosis e.g. alpha-1 antitrypsin deficiency, hemochromatosis, pancreatic disorders including pancreatic cancer; gall bladder disease (PBC, PSC), coronary artery disease, peripheral vascular disease, hypertension, endothelial dysfunction, impaired vascular compliance, congestive heart failure, myocardial infarction, stroke, hemorrhagic stroke, ischemic stroke, pulmonary hypertension, restenosis after angioplasty, intermittent claudication, post-prandial lipemialeft ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, foot ulcerations, ulcerative colitis, hyperapobetalipoproteinemia, Alzheimer's Disease, schizophrenia, impaired cognition, inflammatory bowel disease, ulcerative colitis, Crohn's disease, and irritable bowel syndrome.

In another embodiment, the disclosure provides a method of treating a disease selected from any one or combination of the following: T1D, T2D, insulin resistance, kidney disease, acute kidney disorder, tubular dysfunction, proinflammatory changes to the proximal tubules, adipocyte dysfunction, visceral adipose deposition, obesity, eating disorders, excessive sugar craving, excessive alcohol consumption, dyslipidemia, hyperlipidemmia, hypertriglycerdemia, increased total cholesterol, high LDL cholesterol, high non HDL cholesterol, low HDL cholesterol, NAFLD, MASLD, MetALD, liver steatosis, NASH, MASH, liver fibrosis, cirrhosis, hepatocellular carcinoma, HFK, hypertension, endothelial dysfunction, metabolic syndrome, hyperuricemia, and gout.

Preferred examples of diseases or disorders associated with KHK dysregulation include metabolic syndrome, NAFLD, NASH, MASLD, MASH, MetALD, T2D, hypertriglyceridemia, hypercholesterolemia, DKD, ASH, liver disease arising from hepatocyte stress (e.g. alpha-1 antitrypsin deficiency [AATD], viral hepatitis, or hemochromatosis), viral disease, addictive craving, alcohol use disorder, hyperuricemia, gout, a neurodegenerative disease, and cancer. In some cases, the disease or disorder is NASH or MASH.

KHK or fructokinase catalyzes the first step in fructose metabolism, phosphorylating fructose to fructose-1-phophate (F1P) and depleting intracellular ATP and adenine nucleotide pool. There is no negative feedback mechanism by which F1P inhibits KHK metabolism of fructose, therefore accumulation of F1P is directly related to the amount of fructose either (1) transported into the cell via the GLUT transporters or (2) formed intracellularly from glucose via the polyol pathway, and metabolized via KHK. The accumulation of F1P and depletion of ATP and adenine nucleotide pool cause deleterious consequences in cells, tissues and organs, including oxidative stress, endothelial dysfunction, and metabolic dysregulation. Responses to these insults include lipogenesis and gluconeogenesis, which drive metabolic diseases. A patient experiencing KHK metabolism of fructose that occurs outside of normal parameters risks developing a disease or disorder resulting from the dysregulated state of KHK-mediated fructose metabolism, i.e., a disease or disorder associated with KHK dysregulation.

Non-limiting examples of diseases or disorders associated with excessive fructose intake, increased formation of fructose in hepatocytes via the polyol pathway (e.g. upon osmolar stress as with exposure to alcohol) or KHK dysregulation include metabolic syndrome and metabolic diseases (including type 2 diabetes mellitus (T2D) or hypertriglycerdemia), diseases of the liver [including non-alcoholic fatty liver disease (NAFLD), non-alcoholic steatohepatitis (NASH), alcoholic steatohepatitis (ASH) and arising from hepatocyte stress (e.g. alpha-1 antitrypsin deficiency or hemocrhomatosis)], kidney diseases and disorders including diabetic kidney disease (DKD), addictive craving, alcohol use disorder, hyperuricemia, gout, a neurodegenerative disease (e.g., Parkinson's disease or Alzheimer's disease), and cancer. In some cases, the disease or disorder is NASH.

The terms, “disease”, “disorder”, and “condition” may be used interchangeably here to refer to medical or pathological condition associated with KHK dysregulation.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. The human may be a male or female. In one embodiment, the subject is a non-human animal such as a farm animal (e.g., a horse, cow, pig or sheep), or a pet (e.g., a dog, cat, guinea pig or rabbit). In a preferred embodiment, the subject is a human.

The term “biological sample”, as used herein, includes, without limitation, cell cultures or extracts thereof; biopsied material obtained from a mammal or extracts thereof; blood, saliva, urine, feces, semen, tears, or other body fluids or extracts thereof.

KHK inhibition can be measured by any suitable method known in the art. For example, KHK inhibition in a biological sample (e.g. a cell culture or cell free isolated enzyme) or in humans (e.g. in a subject) can be measured. More specifically, for cell-based assays, in each case cells are cultured in vitro, a test agent is added to the culture, and after a suitable length of time an endpoint is evaluated. Such assays are known in the art.

As used herein, the terms “treat”, “treatment” and “treating” refer to both therapeutic and prophylactic treatments. For example, therapeutic treatments include the reduction or mitigation of the progression, severity and/or duration of diseases or disorders associated with KHK dysregulation, or the amelioration of one or more symptoms (specifically, one or more discernible symptoms) of diseases or disorders associated with KHK dysregulation, resulting from the administration of one or more therapies (e.g., one or more therapeutic agents such as a compound or composition of the disclosure). In specific embodiments, the therapeutic treatment includes the amelioration of at least one measurable physical parameter of diseases or disorders associated with KHK dysregulation. In other embodiments the therapeutic treatment includes the inhibition of the progression of diseases or disorders associated with KHK dysregulation, either physically by, e.g., stabilization of a discernible symptom, physiologically by, e.g., stabilization of a physical parameter, or both. In other embodiments the therapeutic treatment includes the reduction or stabilization of diseases or disorders associated with KHK dysregulation.

The term “chemotherapy” refers to the use of medications, e.g., small molecule drugs (rather than “vaccines”) for treating a disorder or disease.

The terms “prophylaxis” or “prophylactic use” and “prophylactic treatment” as used herein, refer to any medical or public health procedure whose purpose is to prevent, rather than treat or cure a disease. As used herein, the terms “prevent”, “prevention” and “preventing” refer to the reduction in the risk of acquiring or developing a given condition, or the reduction or inhibition of the recurrence of said condition in a subject who is not ill. The term “chemoprophylaxis” refers to the use of medications, e.g. small molecule drugs (rather than “vaccines”) for the prevention of a disorder or disease.

As used herein, prophylactic use includes the use in situations in which the presence of diseases or disorders associated with KHK dysregulation or fructose metabolism, including secondary to excessive consumption of fructose and/or alcohol has been detected. Prophylactic use may also include treating a person who is not ill with diseases or disorders associated with KHK dysregulation or not considered at high risk for complications, in order to reduce the chances of developing diseases or disorders associated with KHK dysregulation.

In some embodiments, the methods of the disclosure are a preventative or “prophylactic” measure to a patient, specifically a human, having a predisposition to complications resulting from diseases or disorders associated with KHK dysregulation.

As used herein, an “effective amount” refers to an amount sufficient to elicit the desired biological response. In the present disclosure the desired biological response is to inhibit KHK in a biological sample or a subject, or to reduce or ameliorate the severity, duration, progression, or onset of a disease or disorder associated with KHK dysregulation, prevent the advancement of a disease or disorder associated with KHK dysregulation, prevent the recurrence, development, onset or progression of a symptom associated with a disease or disorder associated with KHK dysregulation, or enhance or improve the prophylactic or therapeutic effect(s) of another therapy used against diseases or disorders associated with KHK dysregulation. The precise amount of compound administered to a subject will depend on the mode of administration, the type and severity of disease or disorder and on the characteristics of the subject, such as general health, age, sex, body weight and tolerance to drugs. The skilled artisan will be able to determine appropriate dosages depending on these and other factors. When co-administered with other agents, e.g., when co-administered with another medication, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, a safe and effective amount should be assumed. For example, compounds described herein can be administered to a subject in a dosage range from between approximately 0.01 to 100 mg/kg body weight/day for therapeutic or prophylactic treatment.

Generally, dosage regimens can be selected in accordance with a variety of factors including the disease or disorder being treated and the severity of the disease or disorder; the activity of the specific compound employed; the specific composition employed; the age, body weight, general health, sex and diet of the patient; the time of administration, route of administration, and rate of excretion of the specific compound employed; the renal and hepatic function of the subject and the particular compound or salt thereof employed, the duration of the treatment; drugs used in combination or coincidental with the specific compound employed, and like factors well known in the medical arts. The skilled artisan can readily determine and prescribe the effective amount of the compounds described herein required to treat, to prevent, inhibit (fully or partially) or arrest the progress of the disease or disorder.

Dosages of the compounds for uses described herein can range from between about 0.01 to about 100 mg/kg body weight/day, about 0.01 to about 50 mg/kg body weight/day, about 0.1 to about 50 mg/kg body weight/day, or about 1 to about 25 mg/kg body weight/day. It is understood that the total amount per day can be administered in a single dose or can be administered in multiple dosing, such as twice a day (e.g., every 12 hours), three times a day (e.g., every 8 hours), or four times a day (e.g., every 6 hours).

For therapeutic treatment, the compounds described herein can be administered to a patient within, for example, 48 hours (or within 40 hours, or less than 2 days, or less than 1.5 days, or within 24 hours) of onset of symptoms. The compounds described herein can be also administered to a patient beyond this timeline, for example, within two weeks, six months, one year, five years, or ten years of onset of symptoms. The therapeutic treatment can last for any suitable duration, for example, for 5 days, 7 days, 10 days, 14 days, etc. For prophylactic treatment, the compounds described herein can be administered to a patient for any suitable duration, for example, for 7 days, 10 days, 14 days, 20 days, 28 days, 35 days, 42 days, etc.

### Combination Therapy

The compounds described herein can be used in combination therapy, i.e., in conjunction with drugs, or in conjunction with a vaccine.

A safe and effective amount can be achieved in the method or pharmaceutical composition of the disclosure employing a compound of Formula I or Table A, or a pharmaceutically acceptable salt thereof alone or in combination with an additional suitable therapeutic agent, for example, a drug or a vaccine. When “combination therapy” is employed, a safe and effective amount can be achieved using a first amount of a compound of Formula I or Table A, or a pharmaceutically acceptable salt thereof, and a second amount of an additional suitable therapeutic agent (e.g., a drug or vaccine).

In embodiments, the compound of Formula I or Table A, or a pharmaceutically acceptable salt, and the additional therapeutic agent, are each administered in a safe and effective amount (i.e., each in an amount which would be therapeutically effective if administered alone). In other embodiments, the compound of Formula I or Table A, or a pharmaceutically acceptable salt thereof, and the additional therapeutic agent, are each administered in an amount which alone does not provide a therapeutic effect (a sub-therapeutic dose). In yet other embodiments, the compound of Formula I or Table A, or a pharmaceutically acceptable salt thereof can be administered in a safe and effective amount, while the additional therapeutic agent is administered in a sub-therapeutic dose. In still other embodiments, the compound of Formula I or Table A, a pharmaceutically acceptable salt thereof can be administered in a sub-therapeutic dose, while the additional therapeutic agent, is administered in a safe and effective amount.

Nonlimiting examples of additional therapeutic agents that can be administered to a subject comprise anti-diabetic agents, anti-obesity agents, anti-hypertensive agents, anxiolytic agents, antidepressants, agents to treat diabetic nephropathy, agents to treat diabetic neuropathy, cholesterol/lipid modifying agents, calcium channel blockers, cardiac glycosides, diuretics, anti-platelet agents, anti-coagulants, anti-osteoporosis agents, anti-inflammatory agents, mineralocorticoid receptor antagonists, phosphodiesterase inhibitors, anti-ulcer and gastroesophageal reflux disease agents, hormone replacement therapies, fructose transporter inhibitors, aldose reductase inhibitors, xanthine oxidase inhibitors, drugs for treating bile duct or gallbladder diseases (e.g., primary biliary cholangitis or primary sclerosing cholangitis) and viral liver diseases, therapeutics for treating AATD and hemochromatosis, drugs for treating heart failure particularly preserved ejection heart failure, and agents for the treatment of MASH or NASH. Other nonlimiting examples of additional therapeutic agents that can be administered with compounds disclosed herein (e.g., compounds of Formula I or Table A) include those disclosed in U.S. Pat. No. 10,174,007, incorporated herein by reference.

In some cases, compounds of Formula I or Table A, or a pharmaceutically acceptable salt, may be co-administered with one or more anti-diabetic agent selected from the group consisting of metformin, sulfonylureas (e.g., glipizide, glimepiride, glipentide, and tolbutamide), thiazolidinediones or peroxisome proliferator activating receptor gamma (PPARγ) agonists (e.g., pioglitazone), DPP4 inhibitors (e.g., sitagliptin, linagliptin, vildagliptin, and saxagliptin), meglitinides, insulin and insulin analogs or mimetics, and inhibitors of SGLT1 and/or SGLT2 (e.g., dapaglifozin, empaglifiozin, tofoglifiozin, canaglifiozin, ertugliflozin, and sotagliflozin). In some cases, compounds of Formula I or Table A, or a pharmaceutically acceptable salt, may be co-administered with one or more anti-obesity agent selected from the group consisting of amylin analogs (e.g., cagrilintide, pramlintide, AZD6234, LY3841136, amycretin, petrelintide, NN9487, and LY3541105), incretin hormone receptor agonists or modulators (e.g., semaglutide, liraglutide, tirzepatide, survodutide, retatrutide, pemvidutide, VK2735, RGT-075, cagrilintide/semaglutide, danuglipron, PF-0695422, NN9487, NN9541, CT-388, CT-868, CT-996, orforglipron, efinopegdutide, efocipegtrutide, AZD9550, DR10624, NLY01, maridebart cafraglutide, ECC5004, mazdutide, exenatide, dulaglutide, TERN-601, ecnoglutide, and XW-004), melanocortin 4 receptor agonists (e.g., setmelanotide), leptin receptor agonists (e.g., metreleptin and mibavademab), anti-GIPR mAbs, and activin type II receptor antagonists or ligand traps (e.g., bimagrumab, taldefgrobep alfa, trevogrumab, garetosmab, apitegromab, and SRK-439).

In some cases, compounds of Formula I or Table A or a pharmaceutically acceptable salt, may be co-administered with one or more cholesterol or lipid modifying agent selected from the group consisting of HMG-CoA reductase inhibitors (e.g., pravastatin, lovastatin, atorvastatin, rosuvastatin, simvastatin, and fluvastatin), cholesteryl ester transfer protein inhibitors (e.g., obicetrapib and dalcetrapib), ezetirribe, and PCSK9 inhibitors or modulators (e.g., alirocumab, evolocumab, inclisiran, tafolecimab, recaticimab, AZD-0780, VERVE-102, MK-0616). In some cases, compounds of Formula I or Table A, or a pharmaceutically acceptable salt, may be co-administered with one or more agents for the treatment of NAFLD, MASLD, NASH, or MASH selected from the group consisting of FGF21 analogs (e.g. efruxifermin, pegozafermin, BOS-580, B1344, B13006337, NN9500, NN9499, and HEC8843), thyroid hormone beta-receptor agonists (e.g., resmetirom, VK2809, ASC41, TERN-501, and ALG-055009), incretin hormone receptor agonists or modulators, PPAR agonists (e.g., pioglitazone, lanifibranor, PXL-065, and saroglitazaar), FASN inhibitors (e.g., denifanstat), acetyl CoA carboxylase inhibitors (e.g., firsocostat and clesacostat), inhibitors or modulators of DGAT1 and/or DGAT2 (e.g., ervogostat, SNP-610, SNP-630, 10N224, and PF-07202954), inhibitors or modulators of PNPLA3 (e.g., ALN-PNP, AZD-2693, PF-07853578, LY3849891, JNJ-75220795, and AMG609), alpha-1 antitrypsin enzyme replacement therapies, base editing or siRNA or RNAi or antisense therapies for the treatment of alpha-1 antitrypsin deficiency (e.g., fazirsiran, belossiran, ALN-AAT, NTLA-2003, WVE-006, BEAM-302, and KRRO-110), anti-retroviral therapies for the treatment of HCV or HBV, and inhibitors or modulators of HSD17B13 (e.g., rapirosiran, INI-822, ARO-HSD, and AZD-7503).

In some cases, compounds of Formula I or Table A or a pharmaceutically acceptable salt, may be co-administered with the fructose transporter inhibitor which is an inhibitor of GLUT2, GLUT5, or both. In some cases, compounds of Formula I or Table A or a pharmaceutically acceptable salt, may be co-administered with the aldose reductase inhibitor AT-001, AT-003, gavorestat, ranirestat, epalrestat, fidarestat, imirestat, tolrestat, or risarestat.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Coadministration encompasses administration of the first and second amounts of the compounds of the coadministration in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order.

In embodiments, the present disclosure is directed to methods of combination therapy for inhibiting KHK in biological samples or patients, or for treating or preventing diseases or disorders associated with KHK dysregulation in patients using the compounds or pharmaceutical compositions described herein, e.g., a compound of Formula I or Table A, or a pharmaceutically acceptable salt thereof. Accordingly, pharmaceutical compositions also include those comprising a compound as disclosed herein in combination with one or more additional therapeutic or prophylactic agents for treating or preventing a disease or disorder associated with KHK dysregulation.

Methods of use of the compounds and compositions disclosed herein also include combination of chemotherapy with a compound or composition of Formula I or Table A, or a pharmaceutically acceptable salt thereof or with a combination of a compound or composition of this disclosure with another therapeutic or prophylactic agent.

When co-administration involves the separate administration of the first amount of Formula I or Table A, or a pharmaceutically acceptable salt thereof and a second amount of an additional therapeutic agent, the compounds are administered sufficiently close in time to have the desired therapeutic effect For example, the period of time between each administration which can result in the desired therapeutic effect, can range from minutes to hours and can be determined taking into account the properties of each compound such as potency, solubility, bioavailability, plasma half-life and kinetic profile. For example, a compound of Formula I or Table A, or a pharmaceutically acceptable salt thereof and the second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as a compound of the disclosure) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to a subject.

It is understood that the method of co-administration of a first amount of a compound of Formula I or Table A, or a pharmaceutically acceptable salt thereof and a second amount of an additional therapeutic agent can result in an enhanced or synergistic therapeutic effect, wherein the combined effect is greater than the additive effect that would result from separate administration of the first amount of the compound of Formula I or Table A, or a pharmaceutically acceptable salt thereof and the second amount of the additional therapeutic agent.

As used herein, the term “synergistic” refers to a combination of a compound disclosed herein and another therapy (e.g., a prophylactic or therapeutic agent), which is more effective than presumed additive effects of the therapies. A synergistic effect of a combination of therapies (e.g., a combination of prophylactic or therapeutic agents) can permit the use of lower dosages of one or more of the therapies and/or less frequent administration of said therapies to a subject The ability to utilize lower dosages of a therapy (e.g., a prophylactic or therapeutic agent) and/or to administer said therapy less frequently can reduce the toxicity associated with the administration of said therapy to a subject without reducing the efficacy of said therapy in the prevention, management or treatment of a disorder. In addition, a synergistic effect can result in improved efficacy of agents in the prevention, management or treatment of a disorder. Finally, a synergistic effect of a combination of therapies (e.g., a combination of prophylactic or therapeutic agents) may avoid or reduce adverse or unwanted side effects associated with the use of either therapy alone.

The presence of a synergistic effect can be determined using suitable methods for assessing drug interaction. Suitable methods include, for example, the Sigmoid-Emax equation (Holford, N. H. G. and Scheiner, L. B., Clin. Pharmacokinet 6: 429-453 (1981)), the equation of Loewe additivity (Loewe, S, and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied with experimental data to generate a corresponding graph to aid in assessing the effects of the drug combination. The corresponding graphs associated with the equations referred to above are the concentration-effect curve, isobologram curve and combination index curve, respectively.

### Chiral Separations

The compounds described herein can have asymmetric centers and occur as racemates, racemic mixtures, individual diastereomers or enantiomers, with all isomeric forms being included in the present disclosure. Compounds of the present disclosure having a chiral center can exist in and be isolated in optically active and racemic forms. Some compounds can exhibit polymorphism. The present disclosure encompasses racemic, optically-active, polymorphic, or stereoisomeric forms, or mixtures thereof, of a compound of the disclosure, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution. One can either purify the respective compound, then derivatize the compound to form the compounds described herein, or purify the compound themselves.

The stereospecific stereoisomers or diastereomers are typically conveniently prepared by combination of a methylsulfonylpyrimidine compound with a stereospecific substituted azetidine compound resulting in the desired azetidinylpyrimidiine compound. The methylsulfonylpyrimidine and stereospecific azetidine compounds are selected to achieve the desired stereospecific Formula I compound or the azetidinylpyrimidiyl compound can be further derivatized to achieve alternative Formula I compounds. The hydroxymethyl azetidine precursor stereochemistry preparation is known from the literature (e.g., J. Med. Chem. 2020, 63, 13546-13560). The methyl azetidine precursor enantiomer may be obtained from commercial sources. The stereochemistry of the azetidine stereocenters is retained during combination with the methylsulfonylpyrimidine and during further derivatization. Accordingly, the stereochemistry of the desired Formula I stereoisomers and diastereomers is known. Alternative substituted pyrimidine compounds may also be combined with the stereospecific substituted azetidine compounds to achieve the desired Formula I compounds (or further derivatized) as described in the examples (e.g., Ex. 1).

Optically active forms of the compounds can be prepared using any method known in the art, including but not limited to by resolution of the racemic form by recrystallization techniques, by synthesis from optically-active starting materials, by chiral synthesis, or by chromatographic separation using a chiral stationary phase.

Examples of methods to obtain optically active materials include at least the following.
i) physical separation of crystals: a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization: a technique whereby the individual enantiomers are separately crystallized from a solution of the racemate, possible only if the latter is a conglomerate in the solid state;
iii) cocrystallization: a technique whereby the individual enantiomers are crystallized together from a solution of the racemate;
iv) enzymatic resolutions: a technique whereby partial or complete separation of a racemate by virtue of differing rates of reaction for the enantiomers with an enzyme;
v) enzymatic asymmetric synthesis: a synthetic technique whereby at least one step of the synthesis uses an enzymatic reaction to obtain an enantiomerically pure or enriched synthetic precursor of the desired enantiomer;
vi) chemical asymmetric synthesis: a synthetic technique whereby the desired enantiomer is synthesized from an achiral precursor under conditions that produce asymmetry (i.e., chirality) in the product, which can be achieved using chiral catalysts or chiral auxiliaries;
vii) diastereomer separations: a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
viii) first- and second-order asymmetric transformations: a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
ix) kinetic resolutions: this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
x) enantiospecific synthesis from non-racemic precursors a synthetic technique whereby the desired enantiomer is obtained from non-chiral starting materials and where the stereochemical integrity is not or is only minimally compromised over the course of the synthesis;
xi) chiral liquid chromatography: a technique whereby the enantiomers of a racemate are separated in a liquid mobile phase by virtue of their differing interactions with a stationary phase (including but not limited to via chiral HPLC). The stationary phase can be made of chiral material or the mobile phase can contain an additional chiral material to provoke the differing interactions;
xii) chiral gas chromatography: a technique whereby the racemate is volatilized and enantiomers are separated by virtue of their differing interactions in the gaseous mobile phase with a column containing a fixed non-racemic chiral adsorbent phase;
xiii) extraction with chiral solvents: a technique whereby the enantiomers are separated by virtue of preferential dissolution of one enantiomer into a particular chiral solvent;
xiv) transport across chiral membranes: a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including but not limited to simulated moving bed chromatography, is used in one embodiment. A wide variety of chiral stationary phases are commercially available.

The present disclosure will be better understood with reference to the following non-limiting examples.

### Compound Synthesis

The following preparations of compounds of Formula I and intermediates are given to enable those skilled in the art to more dearly understand and to practice the present disclosure. They should not be considered as limiting the scope of the disclosure, but merely as being illustrative and representative thereof.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art having referred to this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

The following LC/MS conditions are referred to in the synthetic examples herein.

Method A: 5-95AB_3.5 min: LC/MS The column used for chromatography was a 5 μm C18 90A, 30×3.0 mm. Detection method was diode array (DAD). MS mode was positive electrospray ionization. MS range was 50-2000. Mobile phase A was 0.04% TFA in water, and mobile phase B was 0.02% TFA in HPLC grade acetonitrile. The gradient was 5-95% B in 3.50 min. 5% B in 0.01 min, 5-95% B (0.01-2.50 min) with a hold at 95% B for 0.50 min, 95-5% B (3.00-3.01 min) with a hold at 5% B for 0.49 min. The flow rate was 1 mL/min (0.01-3.00 min)-1.2 mL/min (3.01-3.50 min).

Method B: 10-100AB_2 min: LC/MS The column used for chromatography was a C18 5 μm, 3.0×30 mm (Sum particles). Detection method was diode array (DAD). MS mode was positive electrospray ionization. MS range was 100-1000. Mobile phase A was 0.04% TFA in water, and mobile phase B was 0.02% TFA in HPLC grade acetonitrile. The gradient was 10-100% B in 1.30 min. 10% B in 0.01 min, 10-100% B (0.01-0.70 min) with a hold at 100% B for 0.60 min. The flow rate was 1.5 mL/min (0.00-1.30 min).

Method C: 5-95AB_2 min: LC/MS The column used for chromatography was a 5 μm C18 90A, 30×3.0 mm. Detection method was diode array (DAD). MS mode was positive electrospray ionization. MS range was 50-2000. Mobile phase A was 0.04% Trifluoroacetic acid in water, and mobile phase B was 0.02% Trifluoroacetic acid in HPLC grade acetonitrile. The gradient was 5-95% B in 1.50 min. 5% B in 0.01 min, 5-95% B (0.01-0.70 min), 95% B for 0.46 min. 95-5% B (1.61-1.50 min) with a hold at 5% B for 0.11 min. The flow rate was 1.5 mL/min.

Method D: 10-100AB_1 min: LC/MS The column used for chromatography was a C18 3.0×30 mm, (Sum particles). Detection method was diode array (DAD). MS mode was positive electrospray ionization. MS range was 50-2000. Mobile phase A was 0.04% TFA in water, and mobile phase B was 0.02% TFA in HPLC grade acetonitrile. The gradient was 10-100% B in 0.90 min. 10% B in 0.01 min, 10-100% B (0.01-0.50 min) with a hold at 100% B for 0.40 min. The flow rate was 2.0 mL/min.

Method E: 5_95AB_6 min-220-254-ELSD: LC/MS The gradient: 5% B in 0.01 min, 5-95% B (0.01-1.60 min), 95-100% B (1.60-2.50 min), 100-5% (2.50-2.52 min) with a hold at 5% B for 0.48 min. The flow rate was 0.8 mL/min. Mobile phase A was 0.037% Trifluoroacetic Acid in water, mobile phase B was 0.018% Trifluoroacetic Acid in acetonitrile. The column used for chromatography was a C18 3.0×30 mm, 2.5 um column (2.5 um particles). Detection methods were diode array (DAD) and evaporative light scattering (ELSD) detection as well as positive electrospray ionization. MS range was 100-1000.

Method F: 5_95CD_6 min-220-254-ELSD: LC/MS The gradient was 5% B in 0.40 min and 5-95% B at 0.40-3.40 min, hold on 95% B for 0.45 min, and then 95-5% B in 0.01 min, the flow rate was 0.8 mL/min. Mobile phase A was H2O+10 mM NH4HCO3, mobile phase B was acetonitrile. The column used for chromatography was a C18 2.1×50 mm column (5 um particles). Detection method was diode array (DAD) and evaporative light scattering (ELSD) detection .MS mode was positive electrospray ionization. MS range was 100-1000.

Method G: 5-95CD_2 min: LC/MS The column used for chromatography was C18 2.1×50 mm, (5 um particles). Detection method was diode array (DAD). MS mode was positive electrospray ionization. MS range was 100-1000. Mobile phase A was 10 mM ammonium bicarbonate in water, and mobile phase B was HPLC grade acetonitrile. The gradient was 5-95% B in 1.50 min. 5% B in 0.01 min, 5-95% B (0.01-0.70 min), 95% B for 0.46 min. 95-5% B (1.61-1.50 min) with a hold at 5% B for 0.11 min. The flow rate was 1.5 mL/min.

Method H: 5-95AB_0.8 min: Mobile phase: Ramp from 5% acetonitrile (0.01875% trifluoroacetic acid) in water (0.0375% trifluoroacetic acid) to 95% acetonitrile in water in 0.60 min, flow rate is set at 2.0 mL/min; then hold at 95% acetonitrile for 0.18 minutes flow rate was set at 2.0 mL/min; returned back to 5% acetonitrile in water and held for 0.02 min. Flow rate was set at 2.0 mL/min. The column temperature was 50° C., and the column was a C18 reverse-phase column of dimensions 2.1×30 mm (5 μm particles).

Method P: 5_95AB_6 min 220-254-ELSD: LC/MS The gradient was 5% B in 0.40 min and 5-95% B in 2.60 min, hold at 95% B for 1.00 min, and then 95-5% B in 0.01 min, the flow rate was 1.0 mL/min. Mobile phase A was 0.04% Trifluoroacetic Acid in water, mobile phase B was 0.02% Trifluoroacetic Acid in acetonitrile. The column used for chromatography was a Luna C18 50′2.0 mm column (5 um particles). Detection methods were diode array (DAD) and evaporative light scattering (ELSD) detection. MS mode was positive electrospray ionization. MS range was 100-1000.

Method R: 10-80AB_10 min: LC/MS The gradient was 10-80% B in 8.00 min with a hold at 80% B for 2.00 min, 80-10% B in 0.01 min, and then held at 10% for 2.99 min (0.5 mL/min flow rate). Mobile phase A was 0.04% Trifluoroacetic Acid in water, mobile phase B was 0.02% Trifluoroacetic Acid in acetonitrile. The column used for chromatography was a Halo AQ-C18 3.0*100 mm column (2.7 um particles). Detection methods are diode array (DAD). MS mode was positive electrospray ionization. MS range was 100-1000.

### Method T:

| Instrument | | Agilent 1260 LC & Agilent 6125C MSD | | |
|---|---|---|---|---|
| Software | | Agilent Open Lab CDS 2.6 | | |
| HPLC | Column | Agilent poroshell 120 EC-C18 | | |
| | | 3.0 * 50 mm, 2.7 μm | | |
| | Mobile Phase | A: 0.0375% TFA in water (v/v) | | |
| | | B: 0.01875% TFA in Acetonitrile (v/v) | | |
| | Gradient | Time (min) | B (%) | Flow (mL/min) |
| | | 0.00 | 05 | 1.0 |
| | | 0.40 | 05 | 1.0 |
| | | 3.00 | 99 | 1.0 |
| | | 4.00 | 99 | 1.0 |
| | | 4.10 | 5 | 1.0 |
| | | 4.50 | 5 | 1.0 |

| | | |
|---|---|---|
| | Post time (min) | Off |
| | Column Temp | 45° C. |
| | Detector | DAD |
| MS | Ionization source | ESI |
| | Drying Gas | N₂ |
| | Drying Gas Flow | 10 (L/min) |
| | Nebulizer Pressure | 40 (psi) |
| | Drying Gas | 350° C. |
| | Temperature | |
| | Capillary Voltage | 3500 (V) Positive |
| | MS Polarity | Positive |
| | MS Mode | Scan |
| | Mass Range | 100-2000 |

### Example 1: Synthesis of Compound A1

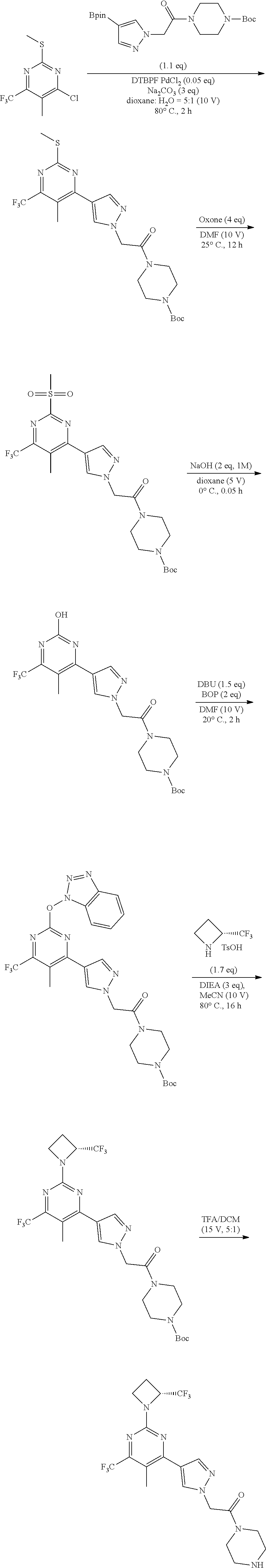

### Synthesis of tert-butyl 4-(2-(4-(4,4,5,5-tetramethyl-1,3,2-dioxaborolan-2-yl)-1H-pyrazol-1-yl)acetyl)piperazine-1-carboxylate

To a solution of 2-[4-(4,4,5,5-tetramethyl-1,3,2-dioxaborolan-2-yl)pyrazol-1-yl]acetic acid (8.50 g, 33.72 mmol, 1 eq) in DMF (85 mL) was added tert-butyl piperazine-1-carboxylate (6.28 g, 33.72 mmol, 1 eq), DIEA (13.07 g, 101.16 mml, 17.62 mL, 3 eq) and HATU (19.23 g, 50.58 mmol, 1.5 eq). The mixture was exchanged with N₂ for 3 times and was stirred at 25° C. for 1 h under N₂ protection. TLC and LCMS showed all starting material was consumed and new spot was formed. The reaction mixture was quenched by addition water (50 mL). The mixture was extracted with ethyl acetate (3×20 mL). The combined organic layers were washed with brine (50 mL), dried over Na₂SO₄, filtered and concentrated under reduced pressure to give a residue. The residue was purified by column on silica gel, eluted with petroleum ether ethyl acetate=3:1 to 0:1, (TLC: petroleum ether:ethyl acetate=0:1, Rf=0.7) to give tert-butyl 4-[2-[4-(4,4,5,5-tetramethyl-1,3,2-dioxaborolan-2-yl)pyrazol-1-yl]acetyl]piperazine-1-carboxylate.

LCMS (ESI+): m/z 421.2 (M+H)⁺, RT: 0.689 min (Method B)

¹H NMR (400 MHz, DMSO-d6) δ ppm 7.84 (s, 1H) 7.56 (s, 1H) 5.16 (s, 2H) 3.35-3.54 (m, 8H) 1.42 (s, 9H) 1.26 (s, 12H)

### Synthesis of tert-butyl 4-[2-[4-[5-methyl-2-methylsulfanyl-6-(trifluoromethyl)pyrimidin-4-yl]pyrazol-1-yl]acetyl]piperazine-1-carboxylate

A solution of 4-chloro-5-methyl-2-methylsulfanyl-6-(trifluoromethyl)pyrimidine (0.5 g, 2.06 mmol, 1 eq) in a mixture of dioxane (4.2 mL) and H₂O (0.8 mL) was added tert-butyl 4-[2-[4-(4,4,5,5-tetramethyl-1,3,2-dioxaborolan-2-yl)pyrazol-1-yl]acetyl]piperazine-1-carboxylate (952.69 mg, 2.27 mmol, 1.1 eq) and then Na₂CO₃ (655.20 mg, 6.18 mmol, 3 eq). The resulting mixture was degassed and then was added DTBPF PdCl₂ (67.15 mg, 103.03 μmol, 0.05 eq). The mixture was exchanged with N₂ for 3 times and was stirred at 80° C. for 2 h under N₂ protection. TLC and LCMS showed all starting material was consumed and new spot was formed. The reaction mixture was quenched with H₂O (10 mL) and then was extracted with ethyl acetate (3×10 mL). The organic layers were combined and concentrated under reduced pressure to give a residue. The residue was purified by column on silica gel, eluted with petroleum ether ethyl acetate=10:1 to 1:1, (TLC: petroleum ether:ethyl acetate=1:2, Rf=0.4) to give tert-butyl 4-[2-[4-[5-methyl-2-methylsulfanyl-6-(trifluoromethyl)pyrimidin-4-yl]pyrazol-1-yl]acetyl]piperazine-1-carboxylate.

LCMS (ESI+): m/z 445.3 (M−55)⁺, RT: 2.077 min (Method A)

¹H NMR (400 MHz, CHLOROFORM-d) δ=8.11 (s, 1H), 8.06 (s, 1H), 5.01 (s, 2H), 3.60-3.45 (m, 4H), 3.43-3.32 (m, 4H), 2.53 (s, 3H), 2.43 (d, J=1.4 Hz, 3H), 1.40 (s, 9H)

### Synthesis of tert-butyl 4-(2-(4-(5-methyl-2-(methylsulfonyl)-6-(trifluoromethyl)pyrimidin-4-yl)-1H-pyrazol-1-yl)acetyl)piperazine-1-carboxylate

A solution of tert-butyl 4-[2-[4-[5-methyl-2-methylsulfanyl-6-(trifluoromethyl)pyrimidin-4-yl]pyrazol-1-yl]acetyl]piperazine-1-carboxylate (0.86 g, 1.72 mmol, 1 eq) in DMF (8.6 mL) was added Oxone (4.23 g, 6.87 mmol, 4 eq). The mixture was stirred at 25° C. for 12 h. LCMS showed the reaction completed. The reaction was poured into sat.aq NaCl (20 mL) and then extracted with ethyl acetate (3×10 mL). The combined organic layers were washed with sat.aq Na₂SO₃ (20 mL) and then the organic layer was dried over Na₂SO₄, concentrated to give tert-butyl 4-[2-[4-[5-methyl-2-methylsulfonyl-6-(trifluoromethyl)pyrimidin-4-yl]pyrazol-1-yl]acetyl]piperazine-1-carboxylate (which was used directly without further purification.

LCMS (ESI+): m/z 555.2 (M+Na)⁺, RT: 0.569 min (Method C)

¹H NMR (400 MHz, CHLOROFORM-d) δ=8.32 (s, 1H), 8.13 (s, 1H), 5.04 (s, 2H), 3.62-3.52 (m, 2H), 3.47 (br d, J=6.9 Hz, 4H), 3.42-3.36 (m, 2H), 3.33 (s, 3H), 2.62 (d, J=1.0 Hz, 3H), 1.41 (s, 9H)

### Synthesis of tert-butyl 4-[2-[4-[2-hydroxy-5-methyl-6-(trifluoromethyl)pyrimidin-4-yl]pyrazol-1-yl]acetyl]piperazine-1-carboxylate

A solution of tert-butyl 4-[2-[4-[5-methyl-2-methylsulfonyl-6-(trifluoromethyl)pyridin-4-yl]pyrazol-1-yl]acetyl]piperazine-1-carboxylate (0.8 g, 1.50 mmol, 1 eq) in dioxane (8 mL) was cooled to 0° C. and then was added NaOH (1 M, 3.00 mL, 2 eq). The mixture was stirred at 0° C. for 30 mins LCMS showed 8% of starting material remained and a major peak with desired product. (TLC: dichlormethane:methanol=3:1, Rf=0.3). The reaction was acidified to pH=3 with HCl (1 M) and then extracted with EtOAc (3×10 mL). The organic was combined and concentrated under reduced pressure to give tert-butyl 4-[2-[4-[2-hydroxy-5-methyl-6-(trifluoromethyl)pyrimidin-4-yl]pyrazol-1-yl]acetyl]piperazine-1-carboxylate which was used directly without further purification.

LCMS (ESI+): m/z 415.1 (M−55)⁺, RT: 0.510 min (Method C)

¹H NMR (400 MHz, CHLOROFORM-d) δ=11.20-10.27 (m, 1H), 8.40 (s, 1H), 7.94-7.87 (m, 1H), 5.13 (s, 2H), 3.63 (s, 4H), 3.54 (br d, J=5.1 Hz, 2H), 3.47 (s, 4H), 3.40 (br d, J=5.1 Hz, 2H), 2.28 (s, 2H), 2.26 (br s, 1H), 1.41 (s, 9H)

### Synthesis of tert-butyl 4-(2-(4-(2-((1H-benzo[d][1,2,3]triazol-1-yl)oxy)-5-methyl-6-(trifluoromethyl)pyrimidin-4-yl)-1H-pyrazol-1-yl)acetyl)piperazine-1-carboxylate

A solution of tert-butyl 4-[2-[4-[2-hydroxy-5-methyl-6-(trifluoromethyl)pyrimidin-4-yl]pyrazol-1-yl]acetyl]piperazine-1-carboxylate (0.5 g, 1.06 mmol, 1 eq) in DMF (5 mL) was added DBU (242.71 mg, 1.59 mmol, 240 μL, 1.5 eq) and BOP (940.13 mg, 2.13 mmol, 2 eq). The mixture was stirred at 20° C. for 2 h. TLC and LCMS showed all starting material was consumed and new spot was formed. The reaction mixture was quenched with H₂O (10 mL). The mixture was extracted with ethyl acetate (3×10 mL). The organic layers was combined and concentrated under reduced pressure to give a residue. The residue was purified by prep-TLC (SiO2, petroleum ether:ethyl acetate=1:5, Rf=0.3) to give tert-butyl 4-[2-[4-[2-(benzotriazol-1-yloxy)-5-methyl-6-(trifluoromethyl)pyrimidin-4-yl]pyrazol-1-yl]acetyl]piperazine-1-carboxylate.

LCMS (ESI+): m/z 610.2 (M+Na)⁺, RT: 0.651 min (Method C)

¹H NMR (400 MHz, CHLOROFORM-d) δ=8.15 (d, J=8.4 Hz, 1H), 7.91 (s, 1H), 7.77 (s, 1H), 7.62-7.43 (m, 3H), 3.69-3.56 (m, 2H), 3.54-3.41 (m, 6H), 2.57 (d, J=1.1 Hz, 3H), 1.50 (s, 9H)

### Synthesis of tert-butyl 4-[2-[4-[5-methyl-6-(trifluoromethyl)-2-[(2R)-2-(trifluoromethyl)azetidin-1-yl]pyrimidin-4-yl]pyrazol-1-yl]acetyl]piperazine-1-carboxylate

To solution of tert-butyl 4-[2-[4-[2-(benzotriazol-1-yloxy)-5-methyl-6-(trifluoromethyl) pyrimidin-4-yl]pyrazol-1-yl]acetyl]piperazine-1-carboxylate (300 mg, 510.59 μmol, 1 eq) in acetonitrile (3 mL) was added DIEA (197.97 mg, 1.53 mmol, 266.81 μL, 3 eq) and (2R)-2-(trifluoromethyl)azetidine (CAS #2554776-09-7; 258.05 mg, 868.01 μmol, 1.7 eq, TsOH). The reaction was stirred at 80° C. for 16 h. LCMS showed around 24% of tert-butyl 4-[2-[4-[2-(benzotriazol-1-yloxy)-5-methyl-6-(trifluoromethyl) pyrimidin-4-yl]pyrazol-1-yl]acetyl]piperazine-1-carboxylate remained and 30% desired product was detected. The reaction was poured into water (5 mL) and extracted with ethyl acetate (2×5 mL). The organic layers were combined and concentrated under reduced pressure to give a residue which was purified by prep-TLC (SiO2, petroleum ether:ethyl acetate=1:5, Rf=0.5) to give tert-butyl 4-[2-[4-[5-methyl-6-(trifluoromethyl)-2-[(2R)-2-(trifluoromethyl)azetidin-1-yl]pyrimidin-4-yl]pyrazol-1-yl]acetyl]piperazine-1-carboxylate (0.04 g, 69.26 μmol, 13.56% yield) as yellowish solid.

LCMS (ESI+): m/z 600.3 (M+Na)⁺, RT: 2.100 min (Method A)

### Synthesis of 2-[4-[5-methyl-6-(trifluoromethyl)-2-[(2R)-2-(trifluoromethyl)azetidin-1-yl]pyrimidin-4-yl]pyrazol-1-yl]-1-piperazin-1-yl-ethanone

A solution of tert-butyl 4-[2-[4-[5-methyl-6-(trifluoromethyl)-2-[(2R)-2-(trifluoromethyl)azetidin-1-yl]pyrimidin-4-yl]pyrazol-1-yl]acetyl]piperazine-1-carboxylate (40 mg, 69.26 μmol, 1 eq) in a mixture of DCM (0.5 mL) and TFA (0.1 ml) was stirred at 25° C. for 1 h. LCMS showed all starting material was consumed and a new peak with desired Ms. The reaction mixture was purified by prep-HPLC, column: Phenomenex Luna C18 75×30 mm (3 um particles), mobile phase: [H₂O (0.1% TFA)-ACN]; gradient 15%-45% B over 8.0 min.) to give 2-[4-[5-methyl-6-(trifluoromethyl)-2-[(2R)-2-(trifluoromethyl)azetidin-1-yl]pyrimidin-4-yl]pyrazol-1-yl]-1-piperazin-1-yl-ethanone.

LCMS (ESI+): m/z 478.2 (M+H)⁺, RT: 2.218 min (Method E)

¹H NMR (400 MHz, METHANOL-d4) δ=8.31 (s, 1H), 8.18 (s, 1H), 5.32 (s, 2H), 4.95-4.90 (m, 1H), 5.00-4.90 (m, 1H), 4.18 (td, J=6.0, 8.4 Hz, 2H), 3.89 (br s, 4H), 3.37 (br s, 2H), 3.29 (br s, 2H), 2.74-2.61 (m, 1H), 2.58-2.51 (m, 1H), 2.48 (d, J=1.5 Hz, 3H)

### Example 2: Synthesis of Compound A2

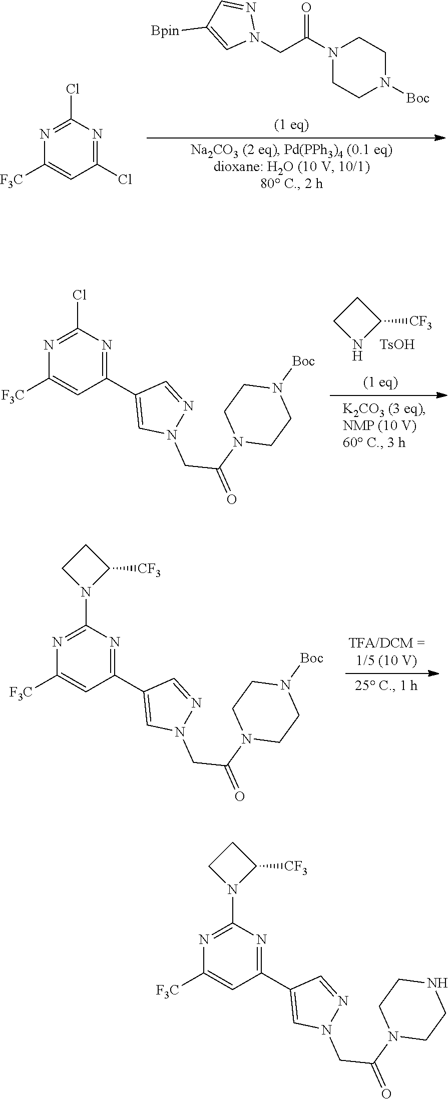

### Synthesis of tert-butyl 4-(2-(4-(2-chloro-6-(trifluoromethyl)pyrimidin-4-yl)-1H-pyrazol-1-yl)acetyl)piperazine-1-carboxylate

To a solution of tert-butyl 4-[2-[4-(4,4,5,5-tetramethyl-1,3,2-dioxaborolan-2-yl)pyrazol-1-yl]acetyl]piperazine-1-carboxylate (5 g, 11.90 mmol, 1 eq) in dioxane (45 mL) and H₂O (4.5 mL) was added 2,4-dichloro-6-(trifluoromethyl)pyrimidine (2.58 g, 11.90 mmol, 2.58 mL, 1 eq), Na₂CO₃ (2.52 g, 23.79 mmol, 2 eq) and Pd(PPh₃)₄ (1.37 g, 1.19 mmol, 0.1 eq). The mixture was exchanged with N₂ for 3 times and was stirred at 80° C. for 2 h under N₂ protection. TLC and LCMS showed all starting material was consumed and new spot formed. The reaction mixture was quenched with water (25 mL). The mixture was extracted with ethyl acetate (3×25 mL). The combined organic layers were washed with brine (30 mL), dried over Na₂SO₄, filtered and concentrated under reduced pressure to give a residue. The residue was purified by column on silica gel, eluted with petroleum ether ethyl acetate=1:0 to 1:5, (TLC: petroleum ether:ethyl acetate=1:1, Rf=0.6) to give tert-butyl 4-(2-(4-(2-chloro-6-(trifluoromethyl)pyrimidin-4-yl)-1H-pyrazol-1-yl)acetyl)piperazine-1-carboxylate.

LCMS (ESI+): m/z 419.1 (M+H)⁺, RT: 0.757 min (Method B)

¹H NMR (400 MHz, CHLOROFORM-d) δ ppm 8.34 (s, 1H) 8.16 (s, 1H) 7.59 (s, 1H) 5.09 (s, 2H) 3.37-3.69 (m, 8H) 1.48 (s, 9H)

### Synthesis of tert-butyl (R)-4-(2-(4-(6-(trifluoromethyl)-2-(2-(trifluoromethyl)azetidin-1-yl)pyrimidin-4-yl)-1H-pyrazol-1-yl)acetyl)piperazine-1-carboxylate

To a solution of tert-butyl 4-(2-(4-(2-chloro-6-(trifluoromethyl)pyrimidin-4-yl)-1H-pyrazol-1-yl)acetyl)piperazine-1-carboxylate (290 mg, 610.69 μmol, 1 eq) in NMP (2.9 mL) was added (R)-2-(trifluoromethyl)azetidine 4-methylbenzenesulfonate (181.55 mg, 619.69 μmol, 1 eq) and K₂CO₃ (253.20 mg, 1.83 mmol, 3 eq). The mixture was stirred at 60° C. for 3 h. LCMS showed all starting material was consumed and a new peak with desired Ms. The reaction mixture was quenched with H₂O (10 mL) and extracted with ethyl acetate (3×5 mL). The combined organic layers were washed with brine (5 mL), dried over Na₂SO₄, filtered and concentrated under reduced pressure to give a residue. The residue was purified by prep-TLC(SiO₂, petroleum ether ethyl acetate=1:1, Rf=0.43) to give tert-butyl (R)-4-(2-(4-(6-(trifluoromethyl)-2-(2-(trifluoromethyl)azetidin-1-yl)pyrimidin-4-yl)-1H-pyrazol-1-yl)acetyl)piperazine-1-carboxylate.

¹H NMR (400 MHz, DMSO-d6) δ=8.45 (s, 1H), 8.23 (s, 1H), 7.59 (s, 1H), 5.27 (s, 2H), 5.12-4.98 (m, 1H), 4.11 (br t, J=7.6 Hz, 2H), 3.55-3.33 (m, 8H), 2.72-2.59 (m, 1H), 2.47-2.36 (m, 1H), 1.42 (s, 9H)

### Synthesis of 1-piperazin-1-yl-2-[4-[6-(trifluoromethyl)-2-[(2R)-2-(trifluoromethyl)azetidin-1-yl]pyrimidin-4-yl]pyrazol-1-yl]ethanone

To a solution of tert-butyl 4-[2-[4-[6-(trifluoromethyl)-2-[(2R)-2-(trifluoromethyl)azetidin-1-yl]pyrimidin-4-yl]pyrazol-1-yl]acetyl]piperazine-1-carboxylate (60 mg, 106.48 μmol, 1 eq) in DCM (0.5 mL) and TFA (0.1 mL). The mixture was stirred at 25° C. for 1 h. LCMS showed all starting material was consumed and a new peak with desired Ms. The reaction was alkalization to pH=7 with saturated sodium bicarbonate aqueous solution and then extracted with ethyl acetate (3×5 mL). The combined organic layers were washed with brine (5 mL), dried over Na₂SO₄, filtered and concentrated under reduced pressure to give a residue. The residue was purified by Prep-HPLC (column: Waters Xbridge BEH C18 100*30 mm*10 um; mobile phase: [water (NH₄HCO₃)-ACN]; gradient: 35%-65% B over 8 min) to give 1-piperazin-1-yl-2-[4-[6-(trifluoromethyl)-2-[(2R)-2-(trifluoromethyl)azetidin-1-yl]pyrimidin-4-yl]pyrazol-1-yl]ethanone.

LCMS (ESI+): m/z 464.1 (M+H)⁺, RT: 2.178 min (Method E)

¹H NMR (400 MHz, METHANOL-d4) δ=8.36 (s, 1H), 8.17 (s, 1H), 7.36 (s, 1H), 5.23 (s, 2H), 5.03-4.91 (m, 1H), 4.19 (td, J=6.4, 8.8 Hz, 2H), 3.63-3.53 (m, 4H), 2.93-2.78 (m, 4H), 2.67 (dtd, J=6.1, 9.1, 11.8 Hz, 1H), 2.51 (tdd, J=6.1, 8.3, 11.9 Hz, 1H)

### Example 3: Synthesis of Compound A3

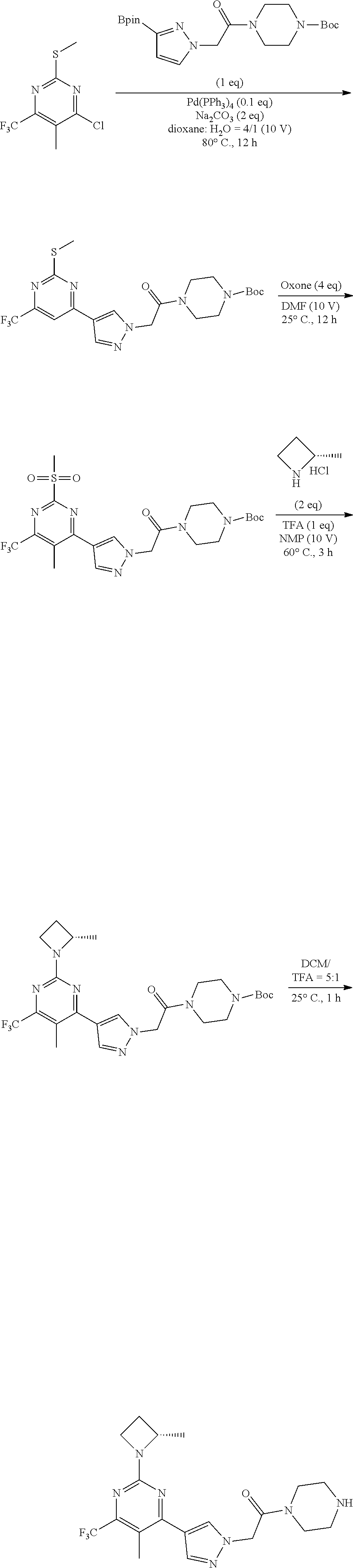

### Synthesis of tert-butyl 4-[2-[4-[5-methyl-2-methylsulfanyl-6-(trifluoromethyl)pyrimidin-4-yl]pyrazol-1-yl]acetyl]piperazine-1-carboxylate

A mixture of tert-butyl 4-[2-[4-(4,4,5,5-tetramethyl-1,3,2-dioxaborolan-2-yl)pyrazol-1-yl]acetyl]piperazine-1-carboxylate (242.50 mg, 576.97 μmol, 1 eq), 4-chloro-5-methyl-2-methylsulfanyl-6-(trifluoromethyl)pyrimidine (140 mg, 576.97 μmol, 1 eq), Pd(PPh₃)₄ (66.67 mg, 57.70 μmol, 0.1 eq), Na₂CO₃ (244.61 mg, 2.31 mmol, 4 eq) in dioxane (1.6 mL) and H₂O (0.4 mL) was degassed and purged with N₂ for 3 times, and then the mixture was stirred at 80° C. for 12 hr under N₂ atmosphere. LCMS showed tert-butyl 4-[2-[4-(4,4,5,5-tetramethyl-1,3,2-dioxaborolan-2-yl)pyrazol-1-yl]acetyl]piperazine-1-carboxylate was consumed completely and one main peak with desired Ms. The reaction mixture was quenched with water (3 mL), and extracted with ethyl acetate (4 mL×3). The combined organic layers were washed with brine (4 mL×2), dried over Na₂SO₄, filtered and concentrated under reduced pressure to give a residue. The residue was purified by column chromatography (SiO₂, petroleum ether/ethyl acetate=1:3, Rf=0.6) to give tert-butyl 4-[2-[4-[5-methyl-2-methylsulfanyl-6-(trifluoromethyl)pyrimidin-4-yl]pyrazol-1-yl]acetyl]piperazine-1-carboxylate.

LCMS (ESI+): m/z 445.1 (M−55)⁺, RT: 0.837 min (Method B)

¹H NMR (400 MHz, DMSO-d6) δ ppm 8.48 (s, 1H) 8.16 (s, 1H) 5.29 (s, 2H) 3.37-3.59 (m, 8H) 2.59 (s, 3H) 2.46 (d, J=1.31 Hz, 3H) 1.42 (s, 9H).

### Synthesis of tert-butyl 4-[2-[4-[5-methyl-2-methylsulfonyl-6-(trifluoromethyl)pyrimidin-4-yl]pyrazol-1-yl]acetyl]piperazine-1-carboxylate

To a solution of tert-butyl 4-[2-[4-[5-methyl-2-methylsulfanyl-6-(trifluoromethyl)pyrimidin-4-yl]pyrazol-1-yl]acetyl]piperazine-1-carboxylate (200 mg, 399.57 μmol, 1 eq) in DMF (2 mL) was added Oxone (982.57 mg, 1.60 mmol, 4 eq). The mixture was stirred at 25° C. for 12 hr. LCMS showed tert-butyl 4-[2-[4-[5-methyl-2-methylsulfanyl-6-(trifluoromethyl)pyrimidin-4-yl]pyrazol-1-yl]acetyl]piperazine-1-carboxylate was consumed completely and one main peak with desired Ms. The reaction solution was filtered and the filtrate was quenched with ice water (3 mL) and extracted with ethyl acetate (6 mL×3). The combined organic layers were washed with brine (10 mL×2), dried over Na₂SO₄, filtered and concentrated under reduced pressure to give a residue which was purified by prep-TLC (SiO₂, petroleum ether/ethyl acetate=1:3, Rf=0.3) to give tert-butyl 4-[2-[4-[5-methyl-2-methylsulfonyl-6-(trifluoromethyl)pyrimidin-4-yl]pyrazol-1-yl]acetyl]piperazine-1-carboxylate.

LCMS (ESI+): m/z 433.1 (M−100)⁺, RT: 0.706 min (Method B)

### Synthesis of tert-butyl 4-[2-[4-[5-methyl-2-[(2S)-2-methylazetidin-1-yl]-6-(trifluoromethyl)pyrimidin-4-yl]pyrazol-1-yl]acetyl]piperazine-1-carboxylate

To a solution of (2S)-2-methylazetidine (CAS #935669-67-3; 80 mg, 743.61 μmol, 2 eq. HCl salt), tert-butyl 4-[2-[4-[5-methyl-2-methylsulfonyl-6-(trifluoromethyl)pyrimidin-4-yl]pyrazol-1-yl]acetyl]piperazine-1-carboxylate (198 mg, 371.81 μmol, 1 eq) in NMP (2 mL) was added TEA (150.49 mg, 1.49 mmol, 207.00 μL, 4 eq). The mixture was stirred at 60° C. for 3 hr. LCMS showed tert-butyl 4-[2-[4-[5-methyl-2-methylsulfonyl-6-(trifluoromethyl) pyrimidin-4-yl]pyrazol-1-yl]acetyl]piperazine-1-carboxylate was consumed completely and one main peak with desired Ms. The reaction mixture was quenched with water (1.5 mL), and extracted with ethyl acetate (2 mL×3). The combined organic layers were washed with brine (2 mL×3), dried over Na₂SO₄, filtered and concentrated under reduced pressure to give a residue which was purified by Prep-HPLC (neutral condition: H₂O (0.05% NH₃H₂O+10 mM NH₄HCO₃); B: ACN) to give tert-butyl 4-[2-[4-[5-methyl-2-[(2S)-2-methylazetidin-1-yl]-6-(trifluoromethyl)pyrimidin-4-yl]pyrazol-1-yl]acetyl]piperazine-1-carboxylate.

LCMS (ESI+): m/z 524.2 (M+H)⁺, RT: 0.898 min (Method B)

### Synthesis of 2-[4-[5-methyl-2-[(2S)-2-methylazetidin-1-yl]-6-(trifluoromethyl)pyrimidin-4-yl]pyrazol-1-yl]-1-piperazin-1-yl-ethanone

A solution of tert-butyl 4-[2-[4-[5-methyl-2-[(2S)-2-methylazetidin-1-yl]-6-(trifluoromethyl)pyrimidin-4-yl]pyrazol-1-yl]acetyl]piperazine-1-carboxylate (50 mg, 95.50 μmol, 1 eq) in DCM (0.42 mL) was added TFA (0.08 mL). The mixture was stirred at 25° C. for 1 hr. LCMS showed tert-butyl 4-[2-[4-[5-methyl-2-[(2S)-2-methylazetidin-1-yl]-6-(trifluoromethyl)pyrimidin-4-yl]pyrazol-1-yl]acetyl]piperazine-1-carboxylate was consumed completely and one main peak with desired Ms. The pH of reaction mixture was adjusted to 7-8 with saturated aq. NaHCO₃. The reaction mixture was quenched with water (1 mL), and extracted with ethyl acetate (1.5 mL×3). The combined organic layers were washed with brine (1.5 mL×3), dried over Na₂SO₄, filtered and concentrated under reduced pressure to give 2-[4-[5-methyl-2-[(2S)-2-methylazetidin-1-yl]-6-(trifluoromethyl)pyrimidin-4-yl]pyrazol-1-yl]-1-piperazin-1-yl-ethanone.

LC/MS (ESI+): m/z 424.2 (M+H)+, RT: 2.806 min (Method F)

¹H NMR (400 MHz, METHANOL-d4) δ ppm 8.25 (s, 1H) 8.09 (s, 1H) 5.25 (s, 2H) 4.45-4.55 (m, 1H) 3.96-4.10 (m, 2H) 3.63 (br s, 4H) 2.85-3.01 (m, 4H) 2.42 (br s, 1H) 2.40 (d, J=1.31 Hz, 3H) 1.98-2.08 (m, 1H) 1.54 (d, J=6.20 Hz, 3H)

The following compounds were made using similar methods described for compounds A1-A3.

| | Calculated | | |
|---|---|---|---|
| Compound | MW | LC/MS | ¹H NMR |
| A4 | 443.86 | m/z 444.00 (M + H)⁺, | 400 MHz, METHANOL-d4 δ = 8.58 (s, |
| | | RT: 2.922 min | 1H), 8.30 (s, 1H), 5.25 (s, 2H), 4.61- |
| | | (Method F) | 4.48 (m, 1H), 4.15-3.99 (m, 2H), 3.63- |
| | | | 3.53 (m, 4H), 2.94-2.77 (m, 4H), 2.57- |
| | | | 2.46 (m, 1H), 2.05 (tdd, J = 6.7, 9.1, 10.8 |
| | | | Hz, 1H), 1.55 (d, J = 6.2 Hz, 3H) |
| A5 | 439.44 | m/z 440.2 (M + 1)⁺, | 400 MHz, METHANOL-d4 δ ppm 8.39 (s, |
| | | RT: 2.798 min | 1 H) 8.18 (s, 1 H) 5.25 (s, 2 H) 4.44- |
| | | (Method F) | 4.54 (m, 1 H) 3.95-4.09 (m, 2 H) 3.72 (s, |
| | | | 3 H) 3.54-3.62 (m, 4 H) 2.79-2.93 (m, |
| | | | 4 H) 2.40-2.51 (m, 1 H) 2.03 (ddt, |
| | | | J = 10.80, 8.87, 7.03, 7.03 Hz, 1 H) 1.54 |
| | | | (d, J = 6.32 Hz, 3 H) |
| A6 | 437.47 | m/z 438.1 (M + H)⁺, | 400 MHz, METHANOL-d4 δ = 8.26 (s, |
| | | RT: 2.922 min | 1H), 8.09 (s, 1H), 5.25 (s, 2H), 4.56- |
| | | (Method F) | 4.43 (m, 1H), 4.12-3.94 (m, 2H), 3.67- |
| | | | 3.54 (m, 4H), 2.96-2.78 (m, 6H), 2.47 |
| | | | (dtd, J = 4.9, 8.5, 10.9 Hz, 1H), 2.03 (tdd, |
| | | | J = 7.0, 9.0, 10.7 Hz, 1H), 1.54 (d, J = 6.2 |
| | | | Hz, 3H), 1.19 (t, J = 7.3 Hz, 3H) |
| A7 | 391.43 | (ESI+): m/z 392.2 | METHANOL-d4, 400 MHz δ 8.31 (s, 1H), |
| | | (M + 1)⁺, | 8.13 (s, 1H), 7.06 (s, 1H), 6.3-6.6 (m, |
| | | RT: 1.634 min | 1H), 5.27 (s, 2H), 4.5-4.6 (m, 1H), 4.0-4.1 |
| | | (Method B) | (m, 2H), 3.86 (br s, 4H), 3.34 (br s, 2H), |
| | | | 3.26 (br s, 2H), 2.4-2.5 (m, 1H), 2.0-2.1 |
| | | | (m, 1H), 1.56 (d, 3H, J = 6.1 Hz) |

### Example 4: Synthesis of Compound A

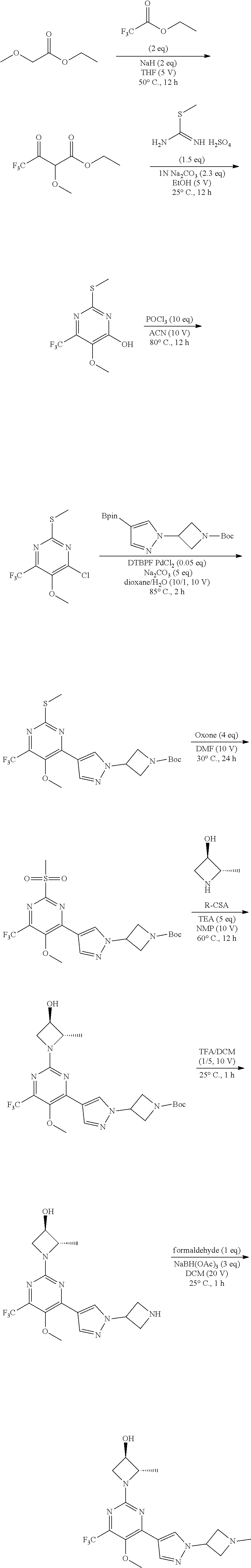

### Synthesis of ethyl 4,4,4-trifluoro-2-methoxy-3-oxo-butanoate

To a solution of ethyl 2-methoxyacetate (35 g, 296.28 mmol, 1 eq) and ethyl 2,2,2-trifluoroacetate (84.19 g, 592.56 mmol, 81.34 mL, 2 eq) in THF (175 mL) was added NaH (23.70 g, 592.56 mmol, 60% purity, 2 eq) at 0° C. The mixture was stirred at 50° C. for 12 h. TLC showed all starting material was consumed and new spot was formed. The reaction mixture was poured into a solution of H₂SO₄/H₂O=1/10 (11 V) and extracted with ethyl acetate (3×150 mL). The combined organic layers were dried over Na₂SO₄, filtered and concentrated under reduced pressure to give a residue. The residue was purified by column on silica gel, eluted with petroleum ether:ethyl acetate=3:1 to 1:1 (TLC: petroleum ether:ethyl acetate=3:1, Rf=0.4) to give ethyl 4,4,4-trifluoro-2-methoxy-3-oxo-butanoate.

¹H NMR (400 MHz, DMSO-d6) δ ppm 4.12-4.20 (m, 2H) 3.91 (s, 1H) 3.31 (s, 3H) 1.20-1.24 (m, 3H)

### Synthesis of 5-methoxy-2-methylsulfanyl-6-(trifluoromethyl)pyrimidin-4-ol

To a solution of ethyl 4,4,4-trifluoro-2-methoxy-3-oxo-butanoate (10 g, 46.70 mmol, 1 eq) in EtOH (50 mL) was added 2-methylisothiourea; sulfuric acid (19.50 g, 70.05 mmol, 1.5 eq) and a solution of Na₂CO₃ (1 M, 107.40 mL, 2.3 eq). The mixture was stirred at 25° C. for 12 h. TLC and LC/MS showed all starting material was consumed and new spot was formed. The reaction was quenched by water (100 mL) and extracted with ethyl acetate (3×150 mL), the combined organic layer was dried over Na₂SO₄, filtered and concentrated under reduced progress to give 5-methoxy-2-methylsulfanyl-6-(trifluoromethyl)pyrimidin-4-ol which was used directly without further purification.

LC/MS (ESI+): m/z 240.9 (M+1)⁺, RT: 0.429 min (Method D)

### Synthesis of 4-chloro-5-methoxy-2-methylsulfanyl-6-(trifluoromethyl)pyrimidine

To a solution of 5-methoxy-2-methylsulfanyl-6-(trifluoromethyl)pyrimidin-4-d (10 g, 41.60 mmol, 1 eq) in ACN (100 mL) was added POCl₃ (63.80 g, 416.3 mmol, 38.80 mL, 10 eq). The mixture was stirred at 80° C. for 12 h. LC/MS showed the reaction completed. The reaction mixture was quenched by addition water (50 mL), and extracted with ethyl acetate (3×100 mL). The combined organic layers were dried over Na₂SO₄, filtered and concentrated under reduced pressure to give a residue. The residue was purified by column on silica gel, eluted with petroleum ether:ethyl acetate=1:1 (TLC: petroleum ether ethyl acetate=1:1, Rf=0.4) to 4-chloro-5-methoxy-2-methylsulfanyl-6-(trifluoromethyl)pyrimidine.

LC/MS (ESI+): m/z 258.8 (M+1)⁺, RT: 0.583 min (Method D)

¹H NMR (400 MHz, DMSO-d6) δ ppm 3.91 (s, 3H) 2.55 (s, 3H)

### Synthesis of tert-butyl 3-[4-[5-methoxy-2-methylsulfanyl-6-(trifluoromethyl)pyrimidin-4-yl]pyrazol-1-yl]azetidine-1-carboxylate

To a solution of 4-chloro-5-methoxy-2-methylsulfanyl-6-(trifluoromethyl)pyrimidine (400 mg, 1546.50 μmol, 1 eq) and tert-butyl 3-[4-(4,4,5,5-tetramethyl-1,3,2-dioxaborolan-2-yl)pyrazol-1-yl]azetidine-1-carboxylate (540.08 mg, 1546.50 μmol, 1 eq) (877399-35-4) in dioxane (4 mL) and H₂O (0.4 mL) was added Na₂CO₃ (819.56 mg, 7.74 mmol, 5 eq) and ditert-butyl(cyclopentyl)phosphane; dichloropalladium; iron (50.40 mg, 77.32 μmol, 0.05 eq). The mixture was stirred at 85° C. for 2 h. TLC and LC/MS showed all starting material was consumed and new spot was formed. The reaction mixture was extracted with ethyl acetate (3×20 mL) and a solution of NaCl (20 mL). The combined organic layers was dried over Na₂SO₄, filtered and concentrated under reduced pressure to give a residue. The residue was purified by column on silica gel, eluted with petroleum ether:ethyl acetate=1:1 (TLC: petroleum ether:ethyl acetate=1:1, Rf=0.4) to give tert-butyl 3-[4-[5-methoxy-2-methylsulfanyl-6-(trifluoromethyl)pyrimidin-4-yl]pyrazol-1-yl]azetidine-1-carboxylate.

LCMS (ESI+): m/z 390 (M−55)⁺, RT: 0.995 min (Method G)

### Synthesis of tert-butyl 3-[4-[5-methoxy-2-methylsulfonyl-6-(trifluoromethyl)pyrimidin-4-yl]pyrazol-1-yl]azetidine-1-carboxylate

To a solution of tert-butyl 3-[4-[5-methoxy-2-methylsulfanyl-6-(trifluoromethyl)pyrimidin-4-yl]pyrazol-1-yl]azetidine-1-carboxylate (300 mg, 673.46 μmol, 1 eq) in DMF (3 mL) was added Oxone (1.66 g, 2.69 mmol, 4 eq). The mixture was stirred at 30° C. for 24 h. TLC and LCMS showed all starting material was consumed and new spot was formed. The reaction mixture was extracted with ethyl acetate (3×20 mL) and a solution of NaCl (50 mL). The combined organic layers was dried over Na₂SO₄, filtered and concentrated under reduced pressure to give tert-butyl 3-[4-[5-methoxy-2-methylsulfonyl-6-(trifluoromethyl)pyrimidin-4-yl]pyrazol-1-yl]azetidine-1-carboxylate which was used directly without further purification.

LCMS (ESI+): m/z 422.0 (M−55)⁺, RT: 0.847 min (Method C)

### Synthesis of tert-butyl 3-[4-[2-[(2S,3R)-3-hydroxy-2-methyl-azetidin-1-yl]-5-methoxy-6-(trifluoromethyl)pyrimidin-4-yl]pyrazol-1-yl]azetidine-1-carboxylate

To a solution of tert-butyl 3-[4-[5-methoxy-2-methylsulfonyl-6-(trifluoromethyl)pyrimidin-4-yl]pyrazol-1-yl]azetidine-1-carboxylate (300 mg, 628.33 μmol, 1 eq) and (2S,3R)-2-methylazetidin-3-ol (200.7 mg, 628.33 mmol, 1 eq, R-CSA salt; synthesized as described in J. Med. Chem. 2020, 63, 13546-13560, intermediate 13/(R)-CSA salt) in NMP (3 mL) was added TEA (317.90 mg, 3.14 mmol, 437.28 μL, 5 eq). The mixture was stirred at 60° C. for 12 h. TLC and LCMS showed all starting material was consumed and new spot formed. The reaction mixture was extracted with ethyl acetate (3×10 mL) and a solution of NaCl (20 mL). The residue was purified by column on silica gel, eluted with petroleum ether:ethyl acetate=1:1 (TLC: petroleum ether:ethyl acetate=1:1, Rf=0.4) to give tert-butyl 3-[4-[2-[(2S,3R)-3-hydroxy-2-methyl-azetidin-1-yl]-5-methoxy-6-(trifluoromethyl)pyrimidin-4-yl]pyrazol-1-yl]azetidine-1-carboxylate.

LCMS (ESI+): m/z 485 (M+1)+, RT: 0.553 min (Method D)

### Synthesis of (2S,3R)-1-[4-[1-(azetidin-3-yl)pyrazol-4-yl]-5-methoxy-6-(trifluoromethyl)pyrimidin-2-yl]-2-methyl-azetidin-3-ol

A solution of tert-butyl 3-[4-[2-[(2S,3R)-3-hydroxy-2-methyl-azetidin-1-yl]-5-methoxy-6-(trifluoromethyl)pyrimidin-4-yl]pyrazol-1-yl]azetidine-1-carboxylate (260 mg, 536.67 μmol, 1 eq) in TFA (0.5 mL) and DCM (2.6 mL) was stirred at 25° C. for 1 h. TLC and LCMS showed all starting material was consumed and new spot was formed. The reaction mixture was concentrated under reduced pressure to give (2S,3R)-1-[4-[1-(azetidin-3-yl)pyrazol-4-yl]-5-methoxy-6-(trifluoromethyl)pyrimidin-2-yl]-2-methyl-azetidin-3-ol.

LCMS (ESI+): m/z 385 (M+1)⁺, RT: 0.353 min (Method D)

### Synthesis of (2S,3R)-1-[5-methoxy-4-[1-(1-methylazetidin-3-yl)pyrazol-4-yl]-6-(trifluoromethyl)pyrimidin-2-yl]-2-methyl-azetidin-3-ol

To a solution of (2S,3R)-1-[4-[l-(azetidin-3-yl)pyrazol-4-yl]-5-methoxy-6-(trifluoromethyl)pyrimidin-2-yl]-2-methyl-azetidin-3-ol (200 mg, 520.35 μmol, 1 eq) in DCM (4 mL) was added formaldehyde (42.23 mg, 520.35 prol, 38.74 μL, 37% purity, 1 eq) and NaBH(OAc)₃ (330.85 mg, 1.56 mmol, 3 eq). The mixture was stirred at 25° C. for 1 h. The reaction mixture was concentrated under reduced pressure to give a residue. The crude product was purified by reversed-phase HPLC (column: Phenomenex Gemini NX-C18 75×30 mm (3 um particles); mobile phase: [H₂O (0.05% NH₃H₂O+10 mM NH₄HCO₃)-ACN]; gradient (25%-50% B over 8.0 min) to give (2S,3R)-1-[5-methoxy-4-[1-(1-methylazetidin-3-yl)pyrazol-4-yl]-6-(trifluoromethyl)pyrimidin-2-yl]-2-methyl-azetidin-3-ol.

LC/MS (ESI+): m/z 399 (M+1)⁺, RT: 2.636 min (Method E)

¹H NMR (400 MHz, METHANOL-d4) δ ppm 8.46 (s, 1H) 8.24 (s, 1H) 5.14 (quin, J=7.12 Hz, 1H) 4.28 (dd, J=9.23, 6.05 Hz, 1H) 4.07-4.22 (m, 2H) 3.88 (t, J=8.07 Hz, 2H) 3.62-3.78 (in. 6H) 2.49 (s, 3H) 1.55 (d. J=6.11 Hz, 3H)

The following compounds were made using similar methods described for compound A8.

| Compound | Calculated MW | LC/MS | ¹H NMR |
|---|---|---|---|
| A9 | 350.37 | (ESI+): m/z 351(M + H)⁺, | 400 MHz, METHANOL-d4 δ ppm |
| | | RT: 2.006 min | 1.54-1.58 (m, 3 H) 2.48 (s, 3 H) 3.62- |
| | | (Method E) | 3.69 (m, 2 H) 3.74-3.79 (m, 1 H) |
| | | | 3.84-3.90 (m, 2 H) 4.16-4.24 (m, 2 |
| | | | H) 4.28-4.35 (m, 1 H) 5.08 (quin, J = |
| | | | 7.07 Hz, 1 H) 6.28-6.58 (m, 1 H) |
| | | | 7.11 (s, 1 H) 8.15 (s, 1 H) 8.44 (s, |
| | | | 1 H) |
| A10 | 402.81 | (ESI+): m/z 403.0 | 400 MHz, METHANOL-d4 δ ppm |
| | | (M + H)⁺, | 8.64 (s, 1 H) 8.35 (s, 1 H) 5.08-5.19 |
| | | RT: 2.810 min | (m, 1 H) 4.29-4.36 (m, 1 H) 4.16- |
| | | (Method F) | 4.25 (m, 2 H) 3.83-3.93 (m, 2 H) |
| | | | 3.76-3.81 (m, 1 H) 3.65-3.72 (m, 2 |
| | | | H) 2.49 (s, 3 H) 1.56 (d, J = 5.96 Hz, |
| | | | 3 H) |
| A11 | 396.42 | (ESI+): m/z 397.1 | 400 MHz, METHANOL-d4 δ = 8.32 |
| | | (M + H)⁺, | (s, 1H), 8.13 (s, 1H), 5.13 (quin, J = |
| | | RT: 2.737 min | 7.1 Hz, 1H), 4.29 (dd, J = 6.0, 9.1 Hz, |
| | | (Method F) | 1H), 4.23-4.11 (m, 2H), 3.92-3.84 |
| | | | (m, 2H), 3.74 (dd, J = 4.8, 9.2 Hz, |
| | | | 1H), 3.71-3.63 (m, 2H), 2.87 (q, J = |
| | | | 7.3 Hz, 2H), 2.49 (s, 3H), 1.55 (d, J = |
| | | | 6.2 Hz, 3H), 1.17 (t, J = 7.3 Hz, 3H) |
| A12 | 382.39 | m/z 383.0 (M + H)⁺, | 400 MHz, METHANOL-d4 δ ppm |
| | | RT: 2.653 min | 8.33 (s, 1 H) 8.13 (s, 1 H) 5.11 (quin, |
| | | (Method F) | J = 7.06 Hz, 1 H) 4.28 (dd, J = 9.06, |
| | | | 6.08 Hz, 1 H) 4.09-4.23 (m, 2 H) |
| | | | 3.82-3.92 (m, 2 H) 3.74 (dd, J = 9.12, |
| | | | 4.83 Hz, 1 H) 3.65-3.71 (m, 2 H) |
| | | | 2.49 (s, 3 H) 2.40 (d, J = 1.31 Hz, 3 H) |
| | | | 1.55 (d, J = 6.08 Hz, 3 H) |

### Example 5: Synthesis of Compound A21 and 3

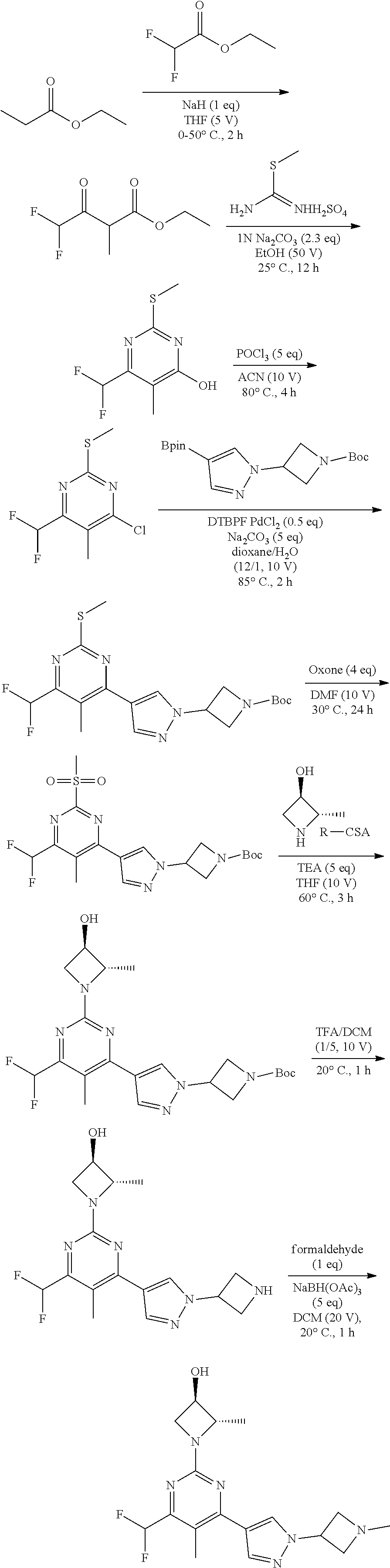

### Synthesis of ethyl 4,4-difluoro-2-methyl-3-oxobutanoate

To a solution of ethyl propanoate (5 g, 48.96 mmol, 5.62 mL, 1 eq) in tetrahydrofuran (25 mL) was added sodium hydride (1.96 g, 48.96 mmol, 60% purity, 1 eq) at 0° C. After addition, the mixture was stirred at this temperature for 5 min, and then ethyl 2,2-difluoroacetate (12.15 g, 97.91 mmol, 2 eq) was added dropwise at 25° C. The resulting mixture was stirred at 50° C. for 2 hrs. TLC indicated starting material was consumed and one major new spot was generated. The reaction mixture was quenched by addition of saturated ammonium chloride solution (50 mL) at 0° C., and then diluted with water (50 mL) and extracted with ethyl acetate (50 mL×3). The combined organic layers were washed with brine (30 mL×2), dried over anhydrous sodium sulfate, filtered and concentrated under reduced pressure to give ethyl 4,4-difluoro-2-methyl-3-oxo-butanoate (7.8 g, 88% yield) as a yellow oil which was used to the next step directly without purification.

¹H NMR (400 MHz, CHLOROFORM-d) δ ppm 5.75-6.08 (m, 1H), 4.10 (q, J=7.13 Hz, 2H), 3.88 (q, J=7.09 Hz, 1H), 1.41 (d, J=7.25 Hz, 3H), 1.27-1.35 (m, 3H).

### Synthesis of 6-(difluoromethyl)-5-methyl-2-(methylthio)pyrimidin-4-ol

To a solution of ethyl 4,4-difluoro-2-methyl-3-oxo-butanoate (5 g, 27.75 mmol, 1 eq) in ethyl alcohol (250 mL) was added 2-methylisothiourea sulfate (1.73 g, 27.75 mmol, 1 eq) and sodium carbonate solution (1 M, 63.84 mL, 2.3 eq) at 25° C. The resulting mixture was stirred at 25° C. for 12 h. LCMS showed starting material was consumed and desired compound was detected. The reaction mixture was concentrated under vacuum to give an oil, which was extracted with ethyl acetate (60 mL×3). The combined organic layers were washed with brine (20 mL×2), dried over anhydrous sodium sulfate, filtered and concentrated under reduced pressure to give a residue. The residue was purified by column chromatography on silica gel, eluting with petroleum ether:ethyl acetate=0:1 to 3:1, (TLC: petroleum ether:ethyl acetate=3:1, R_{f}=0.45) to give 6-(difluoromethyl)-5-methyl-2-methylsulfanyl-pyrimidin-4-ol (3.7 g, 65% yield) as a white solid.

LCMS (ESI+): m/z 207.0 (M+H)⁺, RT: 0.318 min (Method H)

¹H NMR (400 MHz, DMSO-d6) δ ppm 9.85-10.59 (m, 1H), 6.59-6.94 (m, 1H), 2.41 (s, 3H) 1.95 (t, J=2.25 Hz, 3H).

LCMS (ESI+): m/z 207.0 (M+H)⁺, RT: 0.318 min (Method H)

### Synthesis of 4-chloro-6-(difluoromethyl)-5-methyl-2-(methylthio)pyrimidine

To a solution of 6-(difluoromethyl)-5-methyl-2-methylsulfanyl-pyrimidin-4-ol (3.5 g, 16.97 mmol, 1 eq) in acetonitrile (35 mL) was added phosphorus oxychloride (13.01 g, 84.86 mmol, 7.91 mL, 5 eq) at 20° C. The mixture was stirred at 80° C. for 4 h. LCMS showed starting material was consumed completely and desired mass was detected. The reaction mixture was concentrated under vacuum to give an oil, which was extracted with ethyl acetate (60 mL×3). The combined organic layers were washed with brine (20 mL×2), dried over anhydrous sodium sulfate, filtered and concentrated under reduced pressure to give compound 4-chloro-6-(difluoromethyl)-5-methyl-2-methylsulfanyl-pyrimidine (2.5 g, 66% yield) as a grey solid. It was used in the next step directly without purification.

LCMS (ESI+): m/z 225.0 (M+H)⁺, RT: 0.439 min (Method H)

¹H NMR (400 MHz, DMSO-d6) δ ppm 6.96-7.25 (m, 1H), 2.54 (d, J=1.38 Hz, 3H), 2.33 (d, J=1.50 Hz, 3H).

### Synthesis of tert-butyl 3-[4-[6-(difluoromethyl)-5-methyl-2-methylsulfanyl-pyrimidin-4-yl]pyrazol-1-yl]azetidine-1-carboxylate

To a solution of 4-chloro-6-(difluoromethyl)-5-methyl-2-methylsulfanyl-pyrimidine (500 mg, 2.19 mmol, 1 eq) and tert-butyl 3-[4-(4,4,5,5-tetramethyl-1,3,2-dioxaborolan-2-yl)pyrazol-1-yl]azetidine-1-carboxylate (914.05 mg, 2.62 mmol, 1.2 eq) in dioxane (5 mL) and H₂O (0.5 mL) was added Na₂CO₃ (935.4 mg, 10.95 mmol, 5 eq) and DTPBF PdCl₂ (49.8 mg, 111.28 μmol, 0.05 eq) at 25° C. The mixture was stirred at 85° C. for 2 h. LCMS showed starting material was consumed completely and one main peak with desired mass was detected. The reaction mixture was diluted with water (20 mL), extracted with ethyl acetate (40 mL×3). The combined organic layers were washed with brine (100 mL), dried over anhydrous sodium sulfate, filtered and concentrated under reduced pressure. The residue was purified by column chromatography on silica gel, eluted with petroleum ether:ethyl acetate=1:0 to 10:1, (TLC: petroleum ether ethyl acetate=10:1, Rf=0.6) to afford tert-butyl 3-[4-[6-(difluoromethyl)-5-methyl-2-methylsulfanyl-pyrimidin-4-yl]pyrazol-1-yl]azetidine-1-carboxylate (600 mg, 37% yield) as a yellow oil.

LCMS (ESI+): m/z 412.0 (M+H)⁺, RT: 0.495 min (Method H)

¹H NMR (400 MHz, DMSO-d6) δ ppm 8.61 (s, 1H), 8.26 (s, 1H), 7.11 (t, J=53.28 Hz, 1H), 5.30-5.39 (m, 1H), 4.28-4.37 (m, 2H), 4.20 (br s, 2H), 2.57 (s, 3H), 2.44 (s, 3H), 1.41 (s, 9H).

### Synthesis of tert-butyl 3-[4-[6-(difluoromethyl)-5-methyl-2-methylsulfonyl-pyrimidin-4-yl]pyrazol-1-yl]azetidine-1-carboxylate

To a solution of tert-butyl 3-[4-[6-(difluoromethyl)-5-methyl-2-methylsulfanyl-pyrimidin-4-yl]pyrazol-1-yl]azetidine-1-carboxylate (500 mg, 1.22 mmol, 1 eq) in DMF (5 mL) was added Oxone (820 mg, 7.29 mmol, 4 eq) at 20° C. The mixture was stirred at 30° C. for 24 h. LCMS showed reactant was consumed completely and one main peak with desired mass was detected. The reaction mixture was poured into water (15 mL). Solid precipitated out and was collected by filtration. The filter cake was dried by high vacuum to give tert-butyl 3-[4-[6-(difluoromethyl)-5-methyl-2-methylsulfonyl-pyrimidin-4-yl]pyrazol-1-yl]azetidine-1-carboxylate (400 mg, 74% yield) as a yellow solid. It was used for the next step directly without purification.

LCMS (ESI+): m/z 387.9 (M−55)⁺, RT: 0.422 min (Method H)

¹H NMR (400 MHz, DMSO-d6) δ ppm 8.79 (s, 1H), 8.40 (s, 1H), 7.35 (t, J=52.84 Hz, 1H), 5.32-5.42 (m, 1H), 4.29-4.46 (m, 2H), 4.21 (br s, 2H), 3.49 (s, 3H), 2.61 (s, 3H), 1.42 (s, 9H).

### Synthesis of tert-butyl 3-[4-[6-(difluoromethyl)-2-[(2S,3R)-3-hydroxy-2-methyl-azetidin-1-yl]-5-methyl-pyrimidin-4-yl]pyrazol-1-yl]azetidine-1-carboxylate

To a solution of tert-butyl 3-[4-[6-(difluoromethyl)-5-methyl-2-methylsulfonyl-pyrimidin-4-yl]pyrazol-1-yl]azetidine-1-carboxylate (450 mg, 1.01 mmol, 1 eq) in THF (5 mL) was added TEA (513.40 mg, 5.07 mmol, 706.19 μL, 5 eq) and (2S,3R)-2-methylazetidin-3-ol (176.81 mg, 2.03 mml, 2 eq, R-CSA salt synthesized as described in J. Med. Chem. 2020, 63, 13546-13560, intermediate 13/(R)-CSA salt) at 20° C. The mixture was stirred at 60° C. for 3 h. LCMS showed starting material was consumed completely and one main peak with desired mass was detected. The reaction mixture was diluted with water (20 mL) and extracted with ethyl acetate (20 mL×3). The combined organic layers were washed with brine (50 mL), dried over anhydrous sodium sulfate, filtered and concentrated under reduced pressure to give tert-butyl 3-[4-[6-(difluoromethyl)-2-[(2S,3R)-3-hydroxy-2-methyl-azetidin-1-yl]-5-methyl-pyrimidin-4-yl]pyrazol-1-yl]azetidine--carboxylate (400 mg, 887.94 μmol, 87.50% yield) as a white solid. It was used for the next step directly without purification.

LCMS (ESI+): m/z 451.1 (M+H)⁺, RT: 0.463 min (Method H)

¹H NMR (400 MHz, DMSO-d6) δ ppm 8.45 (s, 1H), 8.15 (s, 1H), 6.90 (t, J=53.72 Hz, 1H), 5.28-5.47 (m, 1H), 4.31 (br t, J=8.50 Hz, 2H), 4.16 (br dd, J=8.50, 6.38 Hz, 3H), 4.05 (td, J=10.98, 5.07 Hz, 2H), 3.61 (dd, J=8.76, 4.88 Hz, 1H), 3.49 (s, 1H), 2.33 (s, 3H), 1.44-1.49 (m, 3H), 1.41 (s, 9H).

### Synthesis of (2S,3R)-1-[4-[1-(azetidin-3-yl)pyrazol-4-yl]-6-(difluoromethyl)-5-methyl-pyrimidin-2-yl]-2-methyl-azetidin-3-ol A35

To a solution of tert-butyl 3-[4-[6-(difluoromethyl)-2-[(2S, 3R)-3-hydroxy-2-methyl-azetidin-1-yl]-5-methyl-pyrimidin-4-yl]pyrazol-1-yl]azetidine-1-carboxylate (380 mg, 843.54 μmol, 1 eq) in DCM (5 mL) and TFA (1 mL) at 0° C. The mixture was stirred at 20° C. for 1 hr. LCMS showed starting material was consumed completely and one main peak with desired mass was detected. The reaction mixture was concentrated in a vacuum to give (2S, 3R)-1-[4-[1-(azetidin-3-yl)pyrazol-4-yl]-6-(difluoromethyl)-5-methyl-pyrimidin-2-yl]-2-methyl-azetidin-3-ol (250 mg, 85% yield) as a colorless oil.

LCMS (ESI+): m/z 351.0 (M+H)⁺, RT: 0.281 min (Method H)

¹H NMR (400 MHz, METHANOL-d4) δ 8.31 (s, 1H), 8.14 (s, 1H), 6.58 (t, J=54.3 Hz, 1H), 5.36 (quin, J=7.5 Hz, 1H), 4.28 (dd, J=6.1, 8.9 Hz, 1H), 4.21-4.11 (m, 4H), 4.01-3.93 (m, 2H), 3.73 (dd, J=4.9, 9.0 Hz, 1H), 2.42 (s, 3H), 1.55 (d, J=6.1 Hz, 3H)

### Synthesis of (2S,3R)-1-[4-(difluoromethyl)-5-methyl-6-[1-(1-methylazetidin-3-yl)pyrazol-4-yl]pyrimidin-2-yl]-2-methyl-azetidin-3-ol A21

To a solution of (2S,3R)-1-[4-[1-(azetidin-3-yl)pyrazol-4-yl]-6-(difluoromethyl)-5-methyl-pyrimidin-2-yl]-2-methyl-azetidin-3-ol (250 mg, 710.48 μmol, 1 eq) in DCM (10 mL) was added NaBH(OAc)₃ (752.81 mg, 3.55 mmol, 5 eq) and formaldehyde (61.02 mg, 2.13 mmol, 30%, 1 eq). The mixture was stirred at 20° C. for 1 h. LCMS showed starting material was consumed completely and one main peak with desired mass was detected. The reaction was concentrated in a vacuum. The residue was purified by prep-HPLC (TFA condition; column: Welch Xtimate C18150×25 mm×5 μm; mobile phase: [water (TFA)-ACN]; gradient: 15%-35% B over 2 min) to give (2S,3R)-1-[4-(difluoromethyl)-5-methyl-6-[1-(1-methylazetidin-3-yl)pyrazol-4-yl]pyrimidin-2-yl]-2-methyl-azetidin-3-ol (104 mg, 32% yield) as a white solid.

LCMS (ESI+): m/z 365.3 (M+H)⁺, RT: 1.783 min (Method F)

¹H NMR (400 MHz, METHANOL-d4) δ ppm 8.28 (d, J=12.38 Hz, 2H), 6.58 (t, J=54.34 Hz, 1H), 5.47 (br t, J=6.75 Hz, 1H), 4.35-4.84 (m, 4H), 4.27 (dd, J=8.82, 6.44 Hz, 1H), 4.09-4.23 (m, 2H), 3.72 (dd, J=8.94, 4.94 Hz, 1H), 3.13 (br d, J=1.50 Hz, 3H), 2.41 (s, 3H), 1.55 (d, J=6.25 Hz, 3H).

### Example 6: Synthesis of HCl Salt of A21

A21 free base was dissolved at 50° C. (methanol 2.1 mL (10 volumes), THF 2.1 mL (10 volumes) and acetone 4.2 mL (20 volumes) and then was split into 6 HPLC vials each containing approximately 30 mg of A21 in solution. To approximately 30 mg A21 at 50° C. in solution, 1.1 mol equivalence of HCl (91 μL of 1 M in THF) was added. The sample was left to stir at 50° C. for 1 hour, before cooling to 5° C. at 0.1° C./min overnight. After cooling to 5° C., the resulting clear solution was left uncapped to evaporate at room temperature. The A21 HCl salt suspension was filtered using a PE frit and positive pressure, and the solid was dried under suction. From 1 g of A21 free base, the A21 HCl salt was isolated from 5 volumes methanol using 0.45 μm PTFE filter paper which provided a 86.1% yield.

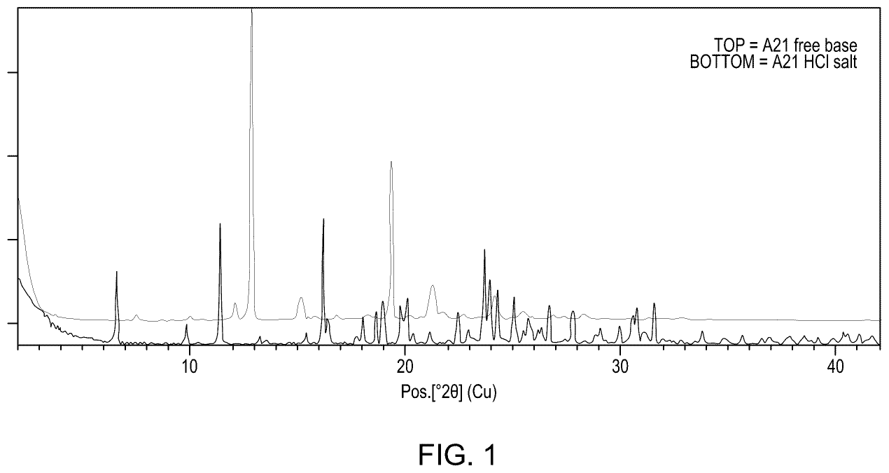

FIG. 1 shows an XPRD diffractogram comparison between A21 Free Base and A21 HCl salt.

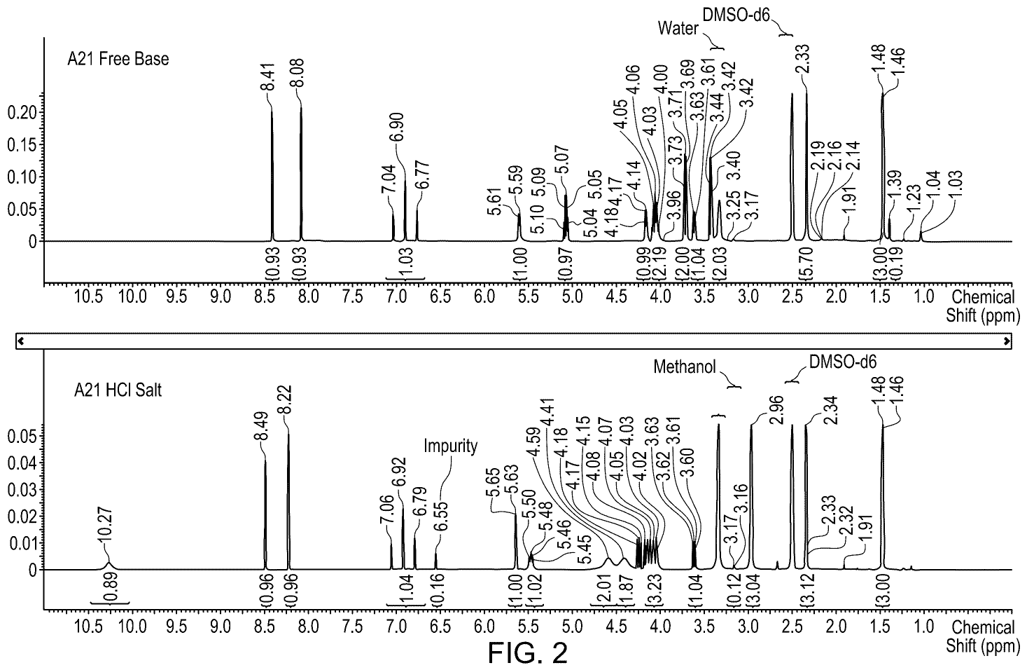

FIG. 2 shows ¹H NMR spectrum comparison between A21 Free Base and HCl salt (DMSO-d6). A summary of peaks is listed below.

A21 free base: 6H peak at 2.33 ppm; A21 HCl salt 2×3H peaks at 2.34 and 2.96 ppm.

A21 free base: 2H peaks at 3.42 and 3.71 ppm; A21 HCl salt: 2H peaks at 4.41 and 4.59 ppm.

A21 free base: 1H peak at 5.07 ppm; A21 HCl salt 1H peak 5.48 ppm.

### Example 7: Synthesis of Compound A41

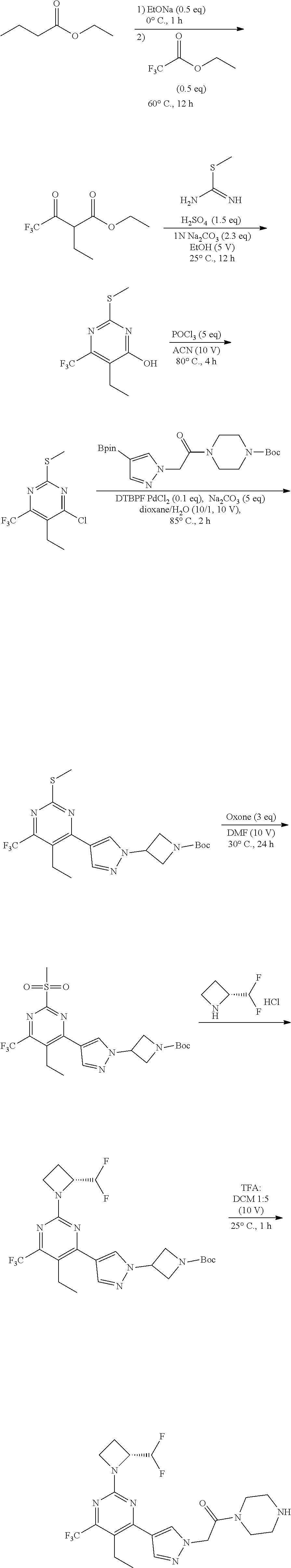

### Synthesis of ethyl 2-ethyl-4,4,4-trifluoro-3-oxo-butanoate

To a dry three-necked flask was added ethyl butanoate (10.79 g, 92.91 mmol, 12.40 mL, 2.2 eq), cooled to 0° C., then EtONa (3.16 g, 46.45 mmol, 1.1 eq) was added. The mixture was stirred for 1 hour at 0-5° C. Then CF₃COOEt (6 g, 42.23 mmol, 5.80 mL, 1 eq) was added dropwise. The mixture was stirred for 12 hours at 65° C. TLC indicated almost of starting material was consumed and one major new spot with larger polarity was detected. The reaction mixture was adjusted to pH=2 with 3N HCl (100 mL) and extracted with DCM (100 mL×3). The combined organic layers were dried over Na₂SO₄, filtered and concentrated under reduced pressure to give ethyl 2-ethyl-4,4,4-trifluoro-3-oxo-butanoate (3.5 g, yield 39%) as a colorless oil.

¹H NMR (400 MHz, CHLOROFORM-d) δ=4.27-4.18 (m, 2H), 3.80-3.69 (m, 1H), 2.02 (quin, J=7.4 Hz, 2H), 1.30-1.23 (m, 3H), 1.02-0.97 (m, 3H)

### Synthesis of 5-ethyl-2-methylsulfanyl-6-(trifluoromethyl)pyrimidin-4-ol

To a solution of 2-methylisothiourea sulfate (6.56 g, 23.57 mmol, 1 eq) in EtOH (25 mL) was added Na₂CO₃ (1 M, 54.20 mL, 2.3 eq). The mixture was stirred at 25° C. for 30 minutes. Then ethyl 2-ethyl-4,4,4-trifluoro-3-oxo-butanoate (5 g, 23.57 mmol, 1 eq) was added. The mixture was stirred at 25° C. for 12 hours. LC-MS showed the starting material was consumed completely and desired mass was detected. The reaction mixture was extracted with ethyl acetate (50 mL×3). The combined organic layers were washed with aqueous NaCl (100 mL), dried over Na₂SO₄, filtered and concentrated under reduced pressure to give 5-ethyl-2-methylsulfanyl-6-(trifluoromethyl)pyrimidin-4-ol (4.5 g, yield 80%) as a yellow oil.

LCMS (ESI+): m/z 238.9 (M+H)⁺, RT: 0.477 min (Method D)

### Synthesis of 4-chloro-5-ethyl-2-methylsulfanyl-6-(trifluoromethyl)pyrimidine

To a solution of 5-ethyl-2-methylsulfanyl-6-(trifluoromethyl)pyrimidin-4-ol (25 g, 104.94 mmol, 1 eq) in ACN (250 mL) was added POCl₃ (160.91 g, 1.05 mol, 97.82 mL, 10 eq). The mixture was stirred at 80° C. for 5 hours. LC-MS showed the starting material was consumed completely and desired mass was detected. The reaction mixture was concentrated under reduced pressure to give a residue. The residue was diluted with H₂O (500 mL) and extracted with ethyl acetate (200 mL×3). The combined organic layers were dried over Na₂SO₄, filtered and concentrated under reduced pressure to give a residue. The residue was purified by flash silica gel chromatography (ISCO®; 120 g SepaFlash® Silica Flash Column, Eluent of 0-5% Ethyl acetate/Petroleum ether, gradient 80 mL/min) to give 4-chloro-5-ethyl-2-methylsulfanyl-6-(trifluoromethyl)pyrimidine (25 g, yield 93%) as a colorless oil.

LCMS (ESI+): m/z 257.0 (M+H)⁺, RT: 0.645 min (Method D)

¹H NMR (400 MHz, CHLOROFORM-d) δ=2.85 (q, J=7.3 Hz, 2H), 2.59 (s, 3H), 1.24 (t, J=7.5 Hz, 3H)

### Synthesis of tert-butyl 4-(2-(4-(5-ethyl-2-(methylthio)-6-(trifluoromethyl)pyrimidin-4-yl)-1H-pyrazol-1-yl)acetyl)iperazine-1-carboxylate

A mixture of 4-chloro-5-ethyl-2-methylsulfanyl-6-(trifluoromethyl)pyrimidine (1 g, 3.90 mmol, 1 eq), tert-butyl 4-[2-[4-(4,4,5,5-tetramethyl-1,3,2-dioxaborolan-2-yl)pyrazol-1-yl]acetyl]piperazine-1-carboxylate (3.28 g, 7.79 mmol, 2 eq), DTBPF PdCl₂ (126.96 mg, 194.80 μmol, 0.05 eq), Na₂CO₃ (2.06 g, 19.48 mmol, 5 eq) in dioxane (10 ml) and H₂O (1 ml) was degassed and purged with N₂ three times, and then the mixture was stirred at 80° C. for 2 hours under an N₂ atmosphere. LCMS showed starting material was consumed and desired mass was detected. The reaction mixture was quenched by addition of water (50 mL), and extracted with ethyl acetate (20 mL×3). The combined organic layers were washed with brine (10 mL×3), dried over Na₂SO₄, filtered and concentrated under reduced pressure to give a residue. The residue was purified by column chromatography (SiO₂, petroleum ether:ethyl acetate=3:1) to give tert-butyl 4-(2-(4-(5-ethyl-2-(methylthio)-6-(trifluoromethyl)pyrimidin-4-yl)-1H-pyrazol-1-yl)acetyl)piperazine-1-carboxylate (800 mg, 39% yield) as a white solid.

¹H NMR (400 MHz, DMSO-d6) δ 7.91 (s, 1H), 7.59 (s, 1H), 5.16 (s, 2H), 3.54-3.32 (m, 8H), 2.82-2.66 (m, 2H), 2.46 (s, 3H), 1.42 (s, 9H), 1.25-1.15 (m, 3H).

### Synthesis of tert-butyl 4-(2-(4-(5-ethyl-2-(methylsulfonyl)-6-(trifluoromethyl)pyrimidin-4-yl)-1H-pyrazol-1-yl)acetyl)piperazine-1-carboxylate

To a solution of tert-butyl 4-(2-(4-(5-ethyl-2-(methylthio)-6-(trifluoromethyl)pyrimidin-4-yl)-1H-pyrazol-1-yl)acetyl)piperazine-1-carboxylate (800 mg, 1.02 mmol, 1 eq) in DMF (8 mL) was added Oxone (1.88 g, 3.05 mmol, 3 eq). The mixture was stirred at 25° C. for 12 hours. LC-MS showed starting material was consumed completely and desired mass was detected. The reaction mixture was quenched by addition of water (10 mL), and extracted with ethyl acetate (12 mL×3). The combined organic layers were washed with brine (10 mL×7), dried over Na₂SO₄, filtered and concentrated under reduced pressure to give a residue. The residue was purified by flash silica gel chromatography (ISCO®; 4 g SepaFlash® Silica Flash Column, Eluent of 70-80% Ethyl acetate/Petroleum ether, gradient 40 mL/min) to give tert-butyl 4-(2-(4-(5-ethyl-2-(methylsulfonyl)-6-(trifluoromethyl)pyrimidin-4-yl)-1H-pyrazol-1-yl)acetyl)piperazine-1-carboxylate (600 mg, yield 71%) as a white solid.

¹H NMR (400 MHz, DMSO-d6) δ 8.66 (s, 1H), 8.29 (s, 1H), 5.35 (s, 2H), 3.44 (br s, 11H), 3.15-2.98 (m, 2H), 1.44-1.42 (m, 9H), 1.28-1.21 (m, 3H)

### tert-butyl-4-[2-[4-[2-[(2R)-2-(difluoromethyl)azetidin-1-yl]-5-ethyl-6-(trifluoromethyl)pyrimidin-4-yl]pyrazol-1-yl]acetyl]piperazine-1-carboxylate

To a solution of (2R)-2-(difluoromethyl)azetidine (CAS #2231665-58-8; 15.20 mg, 105.88 μmol, 1 eq. HCl salt), tert-butyl 4-(2-(4-(5-ethyl-2-(methylsulfonyl)-6-(trifluoromethyl)pyrimidin-4-yl)-1H-pyrazol-1-yl)acetyl)piperazine-1-carboxylate (50 mg, 105.88 μmol, 1 eq) in ACN (1 mL) was added DIEA (68.42 mg, 529.38 μmol, 92.21 μL, 5 eq) and CsF (32.17 mg, 211.75 μmol, 7.82 μL, 2 eq). The mixture was stirred at 130° C. for 12 hours. Five additional vials in 50 mg scale were set up as described above. LC-MS showed starting material was consumed completely and one main peak with desired m/z was detected. The reaction mixture was quenched by addition of water (30 mL), and extracted with ethyl acetate (10 mL×3). The combined organic layers were washed with brine (10 mL), dried over Na₂SO₄, filtered and concentrated under reduced pressure to give tert-butyl 4-[2-[4-[2-[(2R)-2-(difluoromethyl)azetidin-1-yl]-5-ethyl-6-(trifluoromethyl)pyrimidin-4-yl]pyrazol-1-yl]acetyl]piperazine-1-carboxylate (155 mg, 49% yield) which was used in the next step directly.

¹H NMR (400 MHz, DMSO-d6) δ 8.39-8.35 (m, 1H), 8.08 (s, 1H), 6.70-6.18 (m, 1H), 5.28 (s, 2H), 4.08-3.96 (m, 2H), 3.54-3.34 (m, 8H), 2.87-2.76 (m, 2H), 2.45-2.37 (m, 3H), 1.42 (s, 9H), 1.28-1.22 (m, 3H)

### Synthesis of (R)-2-(4-(2-(2-(difluoromethyl)azetidin-1-yl)-5-ethyl-6-(trifluoromethyl)pyrimidin-4-yl)-1H-pyrazol-1-yl)-1-(piperazin-1-yl)ethan-1-one

A solution of tert-butyl 4-[2-[4-[2-[(2R)-2-(difluoromethyl)azetidin-1-yl]-5-ethyl-6-(trifluoromethyl)pyrimidin-4-yl]pyrazol-1-yl]acetyl]piperazine-1-carboxylate (106 mg, 184.81 μmol, 1 eq) in dichloromethane (1 mL) and TFA (0.2 mL) was stirred at 25° C. for 1 hour. LC-MS showed starting material was consumed completely and one main peak with desired m/z was detected. The reaction was adjusted to pH 7-8 with aqueous NaHCO and extracted with ethyl acetate (5 mL×3). The combined organic layers were washed with brine (5 mL), dried over Na₂SO₄, filtered and concentrated under reduced pressure to give a residue. The residue was purified by prep-HPLC (neutral condition: column: Waters Xbridge BEH C18 100*30 mm*10 μm; mobile phase: [H₂O (10 mM NH4HCO₃)-ACN]; gradient 35%-65% B over 8.0 min) to give 2-[4-[2-[(2R)-2-(difluoromethyl)azetidin-1-yl]-5-ethyl-6-(trifluoromethyl)pyrimidin-4-yl]pyrazol-1-yl]-1-piperazin-1-yl-ethanone (40.1 mg, yield 45%) as a white solid.

1H NMR (400 MHz, CD3OD) δ 8.30 (s, 1H), 8.12 (s, 1H), 6.49-6.16 (m, 1H), 5.25 (s, 2H), 4.76-4.58 (m, 1H), 4.17-4.02 (m, 2H), 3.65-3.51 (m, 4H), 2.98-2.86 (m, 4H), 2.85-2.80 (m, 2H), 2.65-2.51 (m, 1H), 2.49-2.37 (m, 1H), 1.21 (t, J=7.4 Hz, 3H)

### Example 8: Synthesis of Compound A46

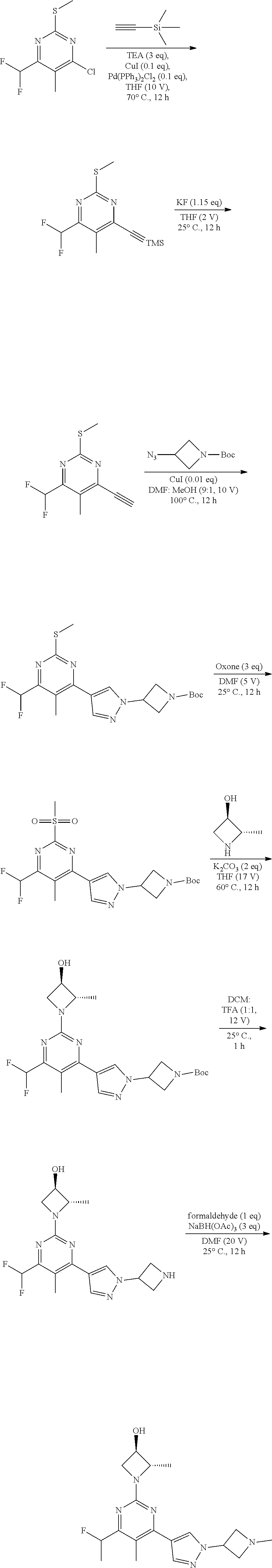

### Synthesis of 4-(difluoromethyl)-5-methyl-2-(methylthio)-6-((trimethylsilyl)ethynyl)pyrimidine

To a solution of 4-chloro-6-(difluoromethyl)-5-methyl-2-methylsulfanyl-pyrimidine (1.4 g, 1 eq) in THF (14 mL) was added ethynyl(trimethyl)silane (1.35 g, 2.2 eq) and TEA (1.89 g, 3 eq) and Pd(PPh₃)₂C₂ (62.49 mg, 89.02 μmol, 0.1 eq) and CuI (16.95 mg, 89.02 μmol, 0.1 eq). The mixture was stirred at 25° C. for 12 h. Several new peaks were shown on LC-MS and 51% of desired compound was detected. The reaction mixture was diluted with water (10 mL) and extracted with EA (5 mL×2). The combined organic layers were washed with aqueous NaCl (5 mL×2), dried over Na₂SO₄, filtered and concentrated under reduced pressure to give a residue. The residue was purified by column chromatography (SiO₂, Petroleum ether/Ethyl acetate=100/0 to 95/5) to give 4-(difluoromethyl)-5-methyl-2-(methylthio)-6-((trimethylsilyl)ethynyl)pyrimidine (0.7 g, 32% yield) as a black oil.

LCMS (ESI+): m/z 287.2 (M+H)⁺, RT: 2.629 min (Method F)

### Synthesis of 4-(difluoromethyl)-6-ethynyl-5-methyl-2-(methylthio)pyrimidine

To a solution of 4-(difluoromethyl)-5-methyl-2-(methylthio)-6-((trimethylsilyl)ethynyl)pyrimidine (0.5 g, 1.75 mmol, 1 eq) in THF (1 mL) was added KF (116.63 mg, 2.01 mmol, 47.03 μL, 1.15 eq). The mixture was stirred at 25° C. for 12 h. Several new peaks were shown on LC-MS and 24% of desired compound was detected. The reaction mixture was diluted with water (5 mL) and extracted with EA (5 mL×2). The combined organic layers were washed with aqueous NaCl (5 mL×2), dried over Na₂SO₄, filtered and concentrated under reduced pressure to give a residue. The residue was purified by prep-TLC (SiO₂, PE:MTBE=3:1) to give 4-(difluoromethyl)-6-ethynyl-5-methyl-2-(methylthio)pyrimidine (0.12 g, 32% yield) as a black-brown solid.

LCMS (ESI+): m/z 215.1 (M+H)⁺, RT: 0.483 min (Method D)

### Synthesis of tert-butyl 3-(4-(6-(difluoromethyl)-5-methyl-2-(methylthio)pyrimidin-4-yl)-1H-1,2,3-triazol-1-yl)azetidine-1-carboxylate

To a solution of 4-(difluoromethyl)-6-ethynyl-5-methyl-2-(methylthio)pyrimidine (0.1 g, 466.78 μmol, 1 eq) in DMF (0.9 mL) and MeOH (0.1 mL) was added CuI (888.98 μg, 4.67 μmol, 0.01 eq) and tert-butyl 3-azido-azetidine-1-carboxylate (92.53 mg, 466.78 μmol, 1 eq). The mixture was stirred at 100° C. for 12 h. Several new peaks were shown on LC-MS and 80% of desired compound was detected. The reaction mixture was diluted with H₂O (3 mL) and extracted with EA 6 mL (3 mL×2). The combined organic layers were washed with aqueous NaCl (5 mL×2), dried over Na₂SO₄, filtered and concentrated under reduced pressure to give a residue. The residue was purified by prep-TLC (SiO₂, PE:EA=1:1) to give tert-butyl 3-(4-(6-(difluoromethyl)-5-methyl-2-(methylthio)pyrimidin-4-yl)-1H-1,2,3-triazol-1-yl)azetidine-1-carboxylate (0.14 g, 73% yield) as a brown solid.

LCMS (ESI+): m/z 413.4 (M+H)+, RT: 0.550 min (Method R)

### Synthesis of tert-butyl 3-(4-(6-(difluoromethyl)-5-methyl-2-(methylsulfonyl)pyrimidin-4-yl)-1H-1,2,3-triazol-1-yl)azetidine-1-carboxylate

To a solution of tert-butyl 3-(4-(6-(difluoromethyl)-5-methyl-2-(methyltho)pyrimidin-4-yl)-1H-1,2,3-triazol-1-yl)azetidine-1-carboxylate (0.09 g, 218.20 μmol, 1 eq) in DMF (0.5 mL) was added Oxone (402.43 mg, 654.61 μmol, 3 eq). The mixture was stirred at 25° C. for 12 h. LC-MS showed 90% of desired compound was detected. The reaction mixture was filtered and the filtrate was concentrated under high vacuum at 40° C. to give tert-butyl 3-(4-(6-(difluoromethyl)-5-methyl-2-(methylsulfonyl)pyrimidin-4-yl)-1H-1,2,3-triazol-1-yl)azetidine-1-carboxylate (0.07 g, 72% yield), which was used in the next step without further purification.

LCMS (ESI+): m/z 467.2 (M+Na)+, RT: 0.456 min (Method D)

### Synthesis of tert-butyl 3-(4-(6-(difluoromethyl)-2-((2S,3R)-3-hydroxy-2-methylazetidin-1-yl)-5-methylpyrimidin-4-yl)-1H-1,2,3-triazol-1-yl)azetidine-1-carboxylate

To a solution of tert-butyl 3-(4-(6-(difluoromethyl)-5-methyl-2-(methylsulfonyl)pyrimidin-4-yl)-1H-1,2,3-triazol-1-yl)azetidine-1-carboxylate (0.06 g, 135.00 μmol, 1 eq) in THF (1 mL) was added K₂CO₃ (37.31 mg, 269.99 μmol, 2 eq) and (2S,3R)-2-methylazetidin-3-ol (39.20 mg, 135.00 μmol, 1 eq). The mixture was stirred at 60° C. for 12 h. LC-MS showed 77% of desired compound was detected. The reaction mixture was diluted with EA (2 mL) and extracted with H₂O (2.5 mL×2). The organic layer was washed with aqueous NaCl (5 mL×2), dried over Na₂SO₄, filtered and concentrated under reduced pressure to give tert-butyl 3-(4-(6-(difluoromethyl)-2-((2S,3R)-3-hydroxy-2-methylazetidin-1-yl)-5-methylpyrimidin-4-yl)-1H-1,2,3-triazol-1-yl)azetidine-1-carboxylate (0.09 g, 74% yield), which was used in the next step without further purification.

LCMS (ESI+): m/z 452.4 (M+H)+, RT: 0.498 min (Method R)

### Synthesis of (2S,3R)-1-(4-(1-(azetidin-3-yl)-1H-1,2,3-triazol-4-yl)-6-(difluoromethyl)-5-methylpyrimidin-2-yl)-2-methylazetidin-3-ol

A mixture of tert-butyl 3-(4-(6-(difluoromethyl)-2-((2S,3R)-3-hydroxy-2-methylazetidin-1-yl)-5-methylpyrimidin-4-yl)-1H-1,2,3-triazol-1-yl)azetidine-1-carboxylate (0.08 g, 177.20 μmol, 1 eq) in DCM (0.5 mL) and TFA (0.5 mL) was stirred at 25° C. for 12 h. LC-MS showed 83% of desired compound was detected. The mixture was concentrated under high vacuum to give (2S,3R)-1-(4-(1-(azetidin-3-yl)-1H-1,2,3-triazol-4-yl)-6-(difluoromethyl)-5-methylpyrimidin-2-yl)-2-methylazetidin-3-ol (0.075 g, 96% yield), which was used into the next step without further purification.

LCMS (ESI+): m/z 352.3 (M+H)+, RT: 0.304 min (Method D)

### Synthesis of (2S,3R)-1-(4-(difluoromethyl)-5-methyl-6-(1-(1-methylazetidin-3-yl)-1H-1,2,3-triazol-4-yl)pyrimidin-2-yl)-2-methylazetidin-3-ol

To a solution of (2S,3R)-1-(4-(1-(azetidin-3-yl)-1H-1,2,3-triazol-4-yl)-6-(difluoromethyl)-5-methylpyrimidin-2-yl)-2-methylazetidin-3-ol (0.02 g, 56.92 μmol, 1 eq) in DMF (0.4 mL) was added NaBH(OAc)₃ (60.32 mg, 284.61 μmol, 5 eq) and formaldehyde (5.70 mg, 56.92 μmol, 5.23 μL, 30% purity, 1 eq). The mixture was stirred at 25° C. for 12 h. LC-MS showed desired compound was detected. The reaction was dried at 40° C. under high vacuum to give a residue which was purified by prep-HPLC (TFA condition) to give (2S,3R)-1-(4-(difluoromethyl)-5-methyl-6-(1-(1-methylazetidin-3-yl)-1H-1,2,3-triazol-4-yl)pyrimidin-2-yl)-2-methylazetidin-3-ol (13 mg, 62% yield) as a white solid.

LCMS (ESI+): m/z 366.1 (M+H)+, RT: 1.823 min Method E

¹H NMR (400 MHz, METHANOL-d4) δ 1.56 (d, J=6.11 Hz, 3H), 2.63 (s, 3H), 3.17 (s, 3H), 3.75 (dd, J=9.11, 4.83 Hz, 1H), 4.11-4.24 (m, 2H), 4.30 (dd, J=9.05, 6.24 Hz, 1H), 4.71-4.85 (m, 4H), 5.75 (br t, J=6.97 Hz, 1H), 6.45-6.95 (m, 1H), 8.40-8.77 (m, 1H)

### Example 9: Synthesis of Compound A31 and A25

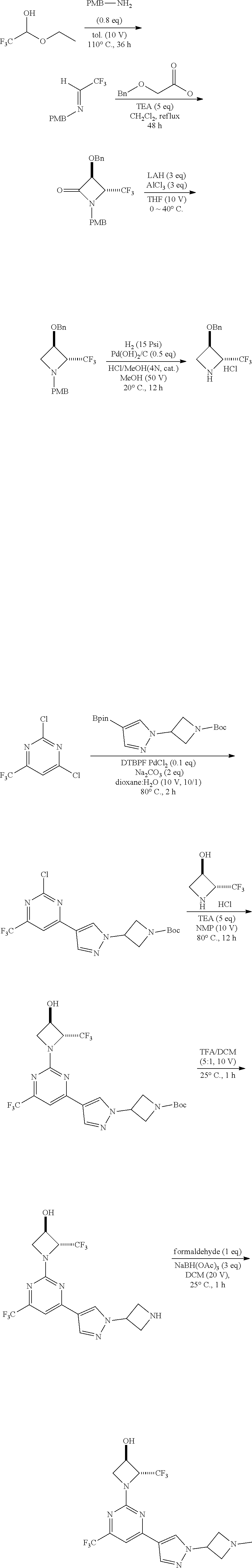

### Synthesis of (E)-2,2,2-trifluoro-N-[(4-methoxyphenyl)methyl]ethanimine

To a solution of 1-ethoxy-2,2,2-trifluoro-ethanol (5 g, 34.70 mmol, 4.10 mL, 1 eq) in toluene (50 mL) was added (4-methoxyphenyl)methanamine (3.81 g, 27.76 mmol, 3.60 mL, 0.8 eq). The reaction was stirred at 110° C. for 36 h. TLC showed desired product was detected. The reaction was concentrated under reduced pressure to give (E)-2,2,2-trifluoro-N-[(4-methoxyphenyl)methyl]ethanimine (7.3 g, 97% yield) as a colorless oil which was used in the next reaction directly.

¹H NMR (400 MHz, CHLOROFORM-d) δ ppm 7.57-7.62 (m, 1H) 7.16-7.22 (m, 2H) 6.89-6.95 (m, 2H) 4.78 (s, 2H) 3.82-3.83 (m, 3H)

### Synthesis of (rac-3S,4R)-3-(benzyloxy)-1-(4-methoxybenzyl)-4-(trifluoromethyl)azetidin-2-one

To a solution of (E)-2,2,2-trifluoro-N-[(4-methoxyphenyl)methyl]ethanimine (7 g, 32.23 mmol, 1 eq) and 2-benzyloxyacetyl chloride (23.80 g, 128.92 mmol, 20.02 mL, 4 eq) in DCM (70 mL) was added TEA (16.31 g, 161.15 mmol, 22.43 mL, 5 eq). The reaction was stirred at 40° C. for 48 h. TLC showed desired product was detected. The reaction mixture was quenched by addition of water (50 mL), and extracted with DCM (70 mL×3). The combined organic layers were dried over Na₂SO₄, filtered and concentrated under reduced pressure to give a residue. The residue was purified by prep-HPLC (column: Phenomenex luna C18 (250*70 mm, 15 um); mobile phase: [H₂O (0.1% TFA)-ACN]; gradient: 53%-73% B over 20.0 min) to give (rac-3S,4R)-3-(benzyloxy)-1-(4-methoxybenzyl)-4- (trifluoromethyl)azetidin-2-one (0.5 g, 4% yield).

LCMS (ESI+): m/z 366.1 (M+1)⁺, RT: 8.215 min (Method R)

### Synthesis of (rac-2R, 3R)-3-(benzyloxy)-1-(4-methoxybenzyl)-2-(trifluoromethyl)azetidine

To a solution of AlCl₃ (437.96 mg, 3.28 mmol, 179.49 μL, 3 eq) in THF (4 mL) was added LAH (2.5 M, 1.31 mL, 3 eq) at 0° C. for 10 min. The reaction was stirred at 40° C. for 30 min. To the reaction was added (rac-3S,4R)-3-benzyloxy-1-[(4-methoxyphenyl)methyl]-4-(trifluoromethyl)azetidin-2-one (400 mg, 1.09 mmol, 1 eq). The reaction was stirred at 20° C. for 3 h. TLC showed desired product was detected. The reaction was quenched with H₂O (10 mL) at 0° C. and extracted with DCM (10 mL×3). The combined organic layers were dried over Na₂SO₄, filtered and concentrated under reduced pressure to give a residue. The residue was purified by prep-TLC (TLC: petroleum ether:ethyl acetate=1:1, Rf=0.4) to give (rac-2R, 3R)-3-(benzyloxy)-1-(4-methoxybenzyl)-2-(trifluoromethyl)azetidine (0.28 g, 73% yield) as a colorless oil.

LCMS (ESI+): m/z 352.1 (M+1)⁺, RT: 0.956 min (Method G)

### Synthesis of (rac-2R,3R)-2-(trifluoromethyl)azetidin-3-ol

To a solution of (rac-2R,3R)-3-(benzyloxy)-1-(4-methoxybenzyl)-2-(trifluoromethyl)azetidine (260 mg, 739.98 μmol, 1 eq) in MeOH (13 mL) and HCl/MeOH (0.01 mL) was added Pd(OH)₂/C (562.38 mg, 739.98 μmol, 20% purity, 1 eq). The reaction was stirred at 20° C. for 12 h under H2 (15 psi). LCMS showed desired product was detected. The resulting product was dissolved in MeOH (30 mL) and filtered. The filtrate was concentrated to give (rac-2R,3R)-2-(trifluoromethyl)azetidin-3-ol (100 mg, 708.76 μmol, 76% yield, HCl salt) as a colorless oil.

¹H NMR (400 MHz, ACETONITRILE-d₃) δ ppm 4.70-5.05 (m, 2H) 4.11-4.23 (m, 1H) 4.00 (br dd, J=10.45, 7.03 Hz, 1H)

### Synthesis of tert-butyl 3-[4-[2-chloro-6-(trifluoromethyl)pyrimidin-4-yl]pyrazol-1-yl]azetidine-1-carboxylate

To a solution of tert-butyl 3-[4-(4,4,5,5-tetramethyl-1,3,2-dioxaborolan-2-yl)pyrazol-1-yl]azetidine-1-carboxylate (804.77 mg, 2.30 mmol, 1 eq) and 2,4-dichloro-6-(trifluoromethyl)pyrimidine (500 mg, 2.30 mmol, 500.00 μL, 1 eq) in dioxane (5 mL) and H₂O (0.5 mL) was added DTBPF PdCl₂ (150.19 mg, 230.44 μmol, 0.1 eq) and Na₂CO₃ (488.48 mg, 4.61 mmol, 2 eq). The reaction was stirred at 80° C. for 2 h. TLC showed desired product was detected. The reaction mixture was diluted with H₂O (10 mL) and extracted with ethyl acetate (20 mL×3). The combined organic layers were dried over Na₂SO₄, filtered and concentrated to give a residue. The residue was purified by prep-TLC (TLC: petroleum ether:ethyl acetate=1:2, Rf=0.3) to give tert-butyl 3-[4-[2-chloro-6-(trifluoromethyl)pyrimidin-4-yl]pyrazol-1-yl]azetidine-1-carboxylate (0.3 g, 32% yield) as a colorless oil.

LCMS (ESI+): m/z 348.0 (M−55)⁺, RT: 0.591 min (Method D)

### Synthesis of tert-butyl 3-(4-(2-((rac-2R,3R)-3-hydroxy-2-(trifluoromethyl)azetidin-1-yl)-6-(trifluoromethyl)pyrimidin-4-yl)-1H-pyrazol-1-yl)azetidine-1-carboxylate

To a solution of (rac-2R,3R)-2-(trifluoromethyl)azetidin-3-ol (48.92 mg, 346.72 μmol, 1 eq) and tert-butyl 3-[4-[2-chloro-6-(trifluoromethyl)pyrimidin-4-yl]pyrazol-1-yl]azetidine-1-carboxylate (140 mg, 346.72 μmol, 1 eq) in THF (1.4 mL) was added TEA (140.34 mg, 1.39 mmol, 193.03 μL, 4 eq). The reaction was stirred at 80° C. for 12 h. TLC showed desired product was detected. The reaction mixture was diluted with H₂O (10 mL) and extracted with ethyl acetate (20 mL×3). The combined organic layers were dried over Na₂SO₄, filtered and concentrated to give a residue which was purified by prep-TLC (TLC: petroleum ether ethyl acetate=1:1, Rf=0.4) to give tert-butyl 3-(4-(2-((rac-2R,3R)-3-hydroxy-2-(trifluoromethyl)azetidin-1-yl)-6-(trifluoromethyl)pyrimidin-4-yl)-1H-pyrazol-1-yl)azetidine-1-carboxylate (91 mg, 52% yield) as a colorless oil.

LCMS (ESI+): m/z 509 (M+H)⁺, RT: 8.48 min (Method R)

### Synthesis of (rac-2R,3R)-1-(4-(1-(azetidin-3-yl)-1H-pyrazol-4-yl)-6-(trifluoromethyl)pyrimidin-2-yl)-2-(trifluoromethyl)azetidin-3-ol A31

A solution of tert-butyl 3-(4-(2-((rac-2R,3R)-3-hydroxy-2-(trifluoromethyl)azetidin-1-yl)-6-(trifluoromethyl)pyrimidin-4-yl)-1H-pyrazol-1-yl)azetidine-1-carboxylate (90 mg, 177.02 μmol, 1 eq) in TFA (0.3 mL) and DCM (1.5 mL) was stirred at 20° C. for 0.5 h. TLC showed desired product was detected. The reaction was concentrated to give (rac-2R,3R)-1-(4-(1-(azetidin-3-yl)-1H-pyrazol-4-yl)-6-(trifluoromethyl)pyrimidin-2-yl)-2-(trifluoromethyl)azetidin-3-ol (70 mg, 97% yield) as a colorless oil.

LCMS (ESI+): m/z 409.1 (M+1)⁺, RT: 0.373 min (Method D)

### Synthesis of (rac-2R,3R)-1-(4-(1-(1-methylazetidin-3-yl)-1H-pyrazol-4-yl)-6-(trifluoromethyl)pyrimidin-2-yl)-2-(trifluoromethyl)azetidin-3-ol A25

To a solution of (rac-2R,3R)-1-(4-(1-(azetidin-3-yl)-1H-pyrazol-4-yl)-6-(trifluoromethyl)pyrimidin-2-yl)-2-(trifluoromethyl)azetidin-3-ol (60 mg, 146.95 μmol, 1 eq) in DCM (1.2 mL) was added NaBH(OAc)₃ (93.43 mg, 440.85 μmol, 3 eq) and formaldehyde (11.93 mg, 146.95 μmol, 10.94 μL, 37% purity, 1 eq). The reaction was stirred at 25° C. for 1 h. TLC showed desired product was detected. The reaction was concentrated to give a residue. The residue was purified by prep-HPLC (column: Phenomenex Luna C18 75*30 mm*3 um; mobile phase: [H₂O (0.1% TFA)-ACN]; gradient 20%-40% B over 8.0 min) to give (rac-2R,3R)-1-(4-(1-(1-methylazetidin-3-yl)-1H-pyrazol-4-yl)-6-(trifluoromethyl)pyrimidin-2- yl)-2-(trifluoromethyl)azetidin-3-ol (40 mg, 64% yield) as a white solid.

LCMS (ESI+): m/z 423.0 (M+1)⁺, RT: 2.779 min (Method F)

¹H NMR (400 MHz, METHANOL-d4) δ ppm 8.51 (s, 1H) 8.22 (s, 1H) 7.42 (s, 1H) 5.08 (t, J=7.03 Hz, 1H) 4.67-4.73 (m, 1H) 4.58-4.66 (m, 1H) 4.42 (dd, J=9.17, 6.72 Hz, 1H) 3.93 (dd, J=9.41, 4.40 Hz, 1H) 3.86 (br t, J=7.95 Hz, 2H) 3.65 (br t, J=7.64 Hz, 2H) 2.48 (s, 3H)

The following compounds were made using similar methods to those described for other compounds disclosed herein, using the appropriate intermediates and reactants.

| Compound | Name | MW | LCMS | 1H NMR |
|---|---|---|---|---|
| A38 | 2-[4-[5-chloro-2-[(2R)-2- | 479.84 | m/z 480.0 | 400 MHz, METHANOL-d4 δ |
| | (difluoromethyl)azetidin-1-yl]- | | (M + H)⁺, RT: | 8.62 (s, 1 H) 8.34 (s, 1 H) |
| | 6-(trifluoromethyl)pyrimidin- | | 2.856 min | 6.33 (ddd, J = 57.56, 55.29, |
| | 4-yl]pyrazol-1-yl]-1-piperazin- | | (Method F) | 1.83 Hz, 1 H) 5.26 (s, 2 H) |
| | 1-yl-ethanone | | | 4.63-4.80 (m, 1 H) 4.04- |
| | | | | 4.22 (m, 2 H) 3.50-3.67 |
| | | | | (m, 4 H) 2.77-2.97 (m, 4 |
| | | | | H) 2.40-2.66 (m, 2 H) |
| A40 | 2-[4-[6-(difluoromethyl)-2- | 455.46 | m/z 456.2 | 400 MHz, METHANOL-d4 δ |
| | [(2R)-2- | | (M + H)⁺, RT: | 8.27 (s, 1 H) 8.10 (s, 1 H) |
| | (difluoromethyl)azetidin-1-yl]- | | 2.718 min | 16.66 (t, J = 54.36 Hz, 1 H) |
| | 5-ethyl-pyrimidin-4- | | (Method F) | 6.15-6.49 (m, 1 H) 5.25 (s, |
| | yl]pyrazol-1-yl]-1-piperazin-1- | | | 2 H) 4.57-4.74 (m, 1 H) |
| | yl-ethanone | | | 4.01-4.17 (m, 2 H) 3.54- |
| | | | | 3.66 (m, 4 H) 2.89-2.98 |
| | | | | (m, 4 H) 2.81-2.88 (m, 2 |
| | | | | H) 2.50-2.63 (m, 1 H) 2.41 |
| | | | | (dtd, J = 11.52, 8.72, 8.72, |
| | | | | 5.72 Hz, 1 H) 1.21 (t, |
| | | | | J = 7.39 Hz, 3 H) |
| A39 | 2-[4-[6-(difluoromethyl)-2- | 445.39 | m/z 446.0 | 400 MHz, METHANOL-d4 δ |
| | [(2R)-2- | | (M + H)⁺, RT: | 2.36-2.48 (m, 1 H) 2.50- |
| | (difluoromethyl)azetidin-1-yl]- | | 2.623 min | 2.65 (m, 1 H) 3.27 (br s, 2 |
| | 5-fluoro-pyrimidin-4- | | (Method F) | H) 3.35 (br s, 2 H) 3.87 (br |
| | yl]pyrazol-1-yl]-1-piperazin-1- | | | s, 4 H) 4.02-4.19 (m, 2 H) |
| | yl-ethanone | | | 4.58-4.76 (m, 1 H) 5.31 (s, |
| | | | | 2 H) 6.15-6.50 (m, 1 H) |
| | | | | 6.73 (t, J = 53.37 Hz, 1 H) |
| | | | | 8.18 (s, 1 H) 8.37 (d, J = 1.47 |
| | | | | Hz, 1 H) |
| A37 | (R)-2-(4-(5-chloro-6- | 461.85 | m/z 462.0 | 400 MHz, METHANOL-d4 δ |
| | (difluoromethyl)-2-(2- | | (M + H)⁺, RT: | 8.60 (s, 1 H) 8.32 (s, 1 H) |
| | (difluoromethyl)azetidin-1- | | 2.669 min | 6.87 (t, J = 53.6, 1 H) 6.49 (t, |
| | yl)pyrimidin-4-yl)-1H-pyrazol- | | (Method F) | J = 54.8, 1 H) 5.26 (s, 2 H) |
| | 1-yl)-1-(piperazin-1-yl)ethan- | | | 4.67-4.80 (m, 1 H) 4.04- |
| | 1-one | | | 4.15 (m, 2 H) 3.57-3.60 |
| | | | | (m, 4 H) 2.82-2.91 (m, 4 |
| | | | | H) 2.45-2.56 (m, 2 H) |
| A43 | (R)-2-(4-(2-(2- | 459.43 | m/z 460.1 | 400 MHz, METHANOL-d4 d |
| | (difluoromethyl)azetidin-1-yl)- | | (M + H)⁺, RT: | 8.28 (s, 1 H) 8.12 (s, 1 H) |
| | 5-methyl-6- | | 2.796 min | 6.49 (t, J = 55.2, 1 H) 5.23 (s, |
| | (trifluoromethyl)pyrimidin-4- | | (Method F) | 2 H) 4.63-4.72 (m, 1 H) |
| | yl)-1H-pyrazol-1-yl)-1- | | | 4.04-4.15 (m, 2 H) 3.57- |
| | (piperazin-1-yl)ethan-1-one | | | 3.60 (m, 4 H) 2.82-2.91 |
| | | | | (m, 4 H) 2.45-2.56 (m, 2 H) |
| A36 | (R)-2-(4-(2-(2- | 475.42 | m/z 476.2 | 400 MHz, METHANOL-d4 δ |
| | (difluoromethyl)azetidin-1-yl)- | | (M + H)⁺, RT: | 8.422 (s, 1 H) 8.266 (s, 1 H) |
| | 5-methoxy-6- | | 2.151 min | 6.455-6.167 ( t, J = 9.6 Hz, |
| | (trifluoromethyl)pyrimidin-4- | | (Method E) | 1 H) 5.313 (s, 2 H) 4.857- |
| | yl)-1H-pyrazol-1-yl)-1- | | | 4.612 (m, 1 H) 4.120- |
| | (piperazin-1-yl)ethan-1-one | | | 4.061 (m, 2 H) 3.87 (br d, |
| | | | | J = 4.65 Hz, 4 H) 3.747 (s, 3 |
| | | | | H) 3.359-3.310 (m, 2 H) |
| | | | | 3.287-3.269 (m, 2 H) |
| | | | | 2.565-22.428 (m, 2 H) |
| A44 | 2-[4-[6-(difluoromethyl)-2- | 457.43 | m/z 458.3 | 400 MHz, METHANOL-d4 δ |
| | [(2R)-2- | | (M + H)⁺, RT: | 8.41 (s, 1H), 8.22 (s, 1H), |
| | (difluoromethyl)azetidin-1-yl]- | | 2.018 min | 6.79 (t, 1H, J = 53.9 Hz), |
| | 5-methoxy-pyrimidin-4- | | (Method T) | 6.1-6.5 (m, 1H), 5.31 (s, |
| | yl]pyrazol-1-yl]-1-piperazin-1- | | | 2H), 4.6-4.7 (m, 1H), 4.0- |
| | yl-ethanone | | | 4.2 (m, 2H), 3.87 (br s, 4H), |
| | | | | 3.76 (s, 3H), 3.35 (br d, |
| | | | | 2H, J = 5.5 Hz), 3.27 (br s, |
| | | | | 2H), 2.5-2.6 (m, 1H), 2.4- |
| | | | | 2.5 (m, 1H) |
| A42 | 2-[4-[6-(difluoromethyl)-2- | 441.43 | (ESI+): m/z 442.3 | 400 MHz, METHANOL-d4 δ |
| | [(2R)-2- | | (M + H)⁺, RT: | 8.27 (s, 1H), 8.13 (s, 1H), |
| | (difluoromethyl)azetidin-1-yl]- | | 2.039 min | 6.76-6.48 (m, 1H), 6.48- |
| | 5-methyl-pyrimidin-4- | | (Method T) | 6.18 (m, 1H), 5.29 (s, 2H), |
| | yl]pyrazol-1-yl]-1-piperazin-1- | | | 4.71-4.59 (m, 1H), 4.13- |
| | yl-ethanone | | | 4.02 (m, 2H), 3.87 (br s, |
| | | | | 4H), 3.35 (br s, 2H), 3.29- |
| | | | | 3.23 (m, 2H), 2.62-2.52 |
| | | | | (m, 1H), 2.44 (s, 3H), 2.43- |
| | | | | 2.37 (m, 1H) |
| A33 | 2-[4-[2-[(2R)-2- | 445.39 | (ESI+): m/z 446.0 | 400 MHz, METHANOL-d4 δ |
| | (difluoromethyl)azetidin-1-yl]- | | (M + H)⁺, RT: | ppm 8.38 (s, 1 H) 8.17 (s, 1 |
| | 6-(trifluoromethyl)pyrimidin- | | 2.734 min | H) 7.28 (s, 1 H) 6.38 (br t, |
| | 4-yl]pyrazol-1-yl]-1-piperazin- | | (Method F) | J = 56.73 Hz, 1 H) 5.22 (s, 2 |
| | 1-yl-ethanone | | | H) 4.61-4.81 (m, 1 H) 4.04- |
| | | | | 4.19 (m, 2 H) 3.53-3.63 |
| | | | | (m, 4 H) 2.79-2.95 (m, 4 |
| | | | | H) 2.53-2.64 (m, 1 H) 2.40- |
| | | | | 2.52 (m, 1 H) |
| A28 | 2-[4-[5-methoxy-6- | 493.41 | (ESI+): m/z 494.1 | 400 MHz, METHANOL-d4 δ |
| | (trifluoromethyl)-2-[(2R)-2- | | (M + H)⁺, RT: | ppm 8.41 (s, 1 H) 8.22 (s, 1 |
| | (trifluoromethyl)azetidin-1- | | 2.287 min | H) 5.32 (s, 2 H) 4.91 (dt, |
| | yl]pyrimidin-4-yllpyrazol-1-yl]- | | (Method P) | J = 9.21, 6.24 Hz, 1 H) 4.15 |
| | 1-piperazin-1-yl-ethanone | | | (ddd, J = 8.67, 6.47, 3.34 Hz, |
| | | | | 2 H) 3.87 (br s, 4 H) 3.77 (s, |
| | | | | 3 H) 3.33-3.41 (m, 2 H) |
| | | | | 3.27 (br d, J = 1.07 Hz, 2 H) |
| | | | | 2.64 (dtd, J = 11.80, 9.00, |
| | | | | 9.00, 6.20 Hz, 1 H) 2.42- |
| | | | | 2.56 (m, 1 H) |
| A24 | 2-[4-[6-(difluoromethyl)-5- | 405.45 | (ESI+): m/z 406.1 | 400 MHz, METHANOL-d4 δ |
| | methyl-2-[(2S)-2- | | (M + H)⁺, RT: | ppm 8.23 (s, 1 H) 8.10 (s, 1 |
| | methylazetidin-1-yl]pyrimidin- | | 2.035 min | H) 6.57 (t, J = 54.47 Hz, 1 H) |
| | 4-yl]pyrazol-1-yl]-1-piperazin- | | (Method F) | 5.29 (s, 2 H) 4.45-4.52 (m, |
| | 1-yl-ethanone | | | 1 H) 3.94-4.10 (m, 2 H) |
| | | | | 3.87 (br d, J = 1.75 Hz, 4 H) |
| | | | | 3.35 (br s, 2 H) 3.27 (br d, |
| | | | | J = 2.63 Hz, 2 H) 2.44-2.52 |
| | | | | (m, 1 H) 2.42 (s, 3 H) 2.03 |
| | | | | (ddt, J = 8.91, 6.97, 1.94, |
| | | | | 1.94 Hz, 1 H) 1.54 (d, |
| | | | | J = 6.25 Hz, 3 H) |
| A29 | 2-[4-[5-chloro-6- | 497.83 | m/z 498.1 | 400 MHz, METHANOL-d4 δ |
| | (trifluoromethyl)-2-[(2R)-2- | | (M + H)⁺, RT: | ppm 8.64 (s, 1 H) 8.38 (s, 1 |
| | (trifluoromethyl)azetidin-1- | | 2.242 min | H) 5.34 (s, 2 H) 4.93-5.05 |
| | yllpyrimidin-4-yl]pyrazol-1-yl]- | | (Method E) | (m, 1 H) 4.10-4.30 (m, 2 |
| | 1-piperazin-1-yl-ethanone | | | H) 3.89 (br s, 4 H) 3.37 (br |
| | | | | d, J = 2.08 Hz, 2 H) 3.29 (br |
| | | | | d, J = 4.52 Hz, 2 H) 2.63- |
| | | | | 2.82 (m, 1 H) 2.41-2.61 |
| | | | | (m, 1 H) |
| A20 | 2-[4-[6-(difluoromethyl)-2- | 445.40 | m/z 446.1 | 400 MHz, METHANOL-d4 δ |
| | [(2R)-2- | | (M + H)⁺, RT: | ppm 8.33 (d, J = 2.32 Hz, 1 |
| | (trifluoromethyl)azetidin-1- | | 2.081 min | H) 8.16 (d, J = 2.32 Hz, 1 H) |
| | yl]pyrimidin-4-yl\|pyrazol-1-yl]- | | (Method E) | 7.27 (s, 1H) 6.34-6.63 (t, |
| | 1-piperazin-1-yl-ethanone | | | J = 2.32 Hz, 1 H) 5.27 (s, 2 |
| | | | | H) 4.86-4.97 (m, 1 H) 4.13- |
| | | | | 4.20 (m, 2 H) 3.86 (br d, |
| | | | | J = 4.65 Hz, 4 H) 3.34-3.42 |
| | | | | (m, 2 H) 3.21-3.29 (m, 2 |
| | | | | H) 2.62-2.75 (m, 1 H) |
| | | | | 2.44-2.57 (m, 1 H) |
| A18 | (R)-2-(4-(6-(difluoromethyl)- | 459.43 | m/z 460.0 | 400 MHz, METHANOL-d4 δ |
| | 5-methyl-2-(2- | | (M + H)⁺, RT: | ppm 8.11-8.18 (m, 1 H) |
| | (trifluoromethyl)azetidin-1- | | 0.353 min | 6.46-6.78 (m, 1 H) 5.28- |
| | yl)pyrimidin-4-yl)-1H-pyrazol- | | (Method E) | 5.31 (m, 2 H) 4.87-4.96 |
| | 1-yl)-1-(piperazin-1-yl)ethan- | | | (m, 1 H) 4.10-4.19 (m, 2 |
| | 1-one | | | H) 3.83-3.91 (m, 4 H) 3.32- |
| | | | | 3.41 (m, 2 H) 3.27 (br s, 2 |
| | | | | H) 2.57-2.68 (m, 1 H) 2.48- |
| | | | | 2.56 (m, 1 H) 8.24-8.29 |
| | | | | (m, 1 H) 2.45-2.48 (m, 3 H) |
| A17 | 2-[4-[5-cyclopropyl-2-[(2R)-2- | 485.46 | m/z 486.3 | METHANOL-d4, 400 MHz δ |
| | (difluoromethyl)azetidin-1-yl]- | | (M + H)⁺, RT: | 8.34 (s, 1H), 8.18 (s, 1H), |
| | 6-(trifluoromethyl)pyrimidin- | | 0.330 min | 6.31 (br t, 1H, J = 56.6 Hz), |
| | 4-yl]pyrazol-1-yl]-1-piperazin- | | (Method E) | 5.29 (s, 2H), 4.6-4.7 (m, |
| | 1-yl-ethanone | | | 1H), 4.0-4.2 (m, 2H), 3.87 |
| | | | | (br s, 4H), 3.35 (br s, 2H), |
| | | | | 3.27 (br s, 2H), 2.5-2.6 (m, |
| | | | | 1H), 2.4-2.5 (m, 1H), 2.01 |
| | | | | (br d, 1H, J = 5.8 Hz), 1.08 |
| | | | | (br d, 2H, J = 8.3 Hz), 0.39 |
| | | | | (br dd, 2H, J = 5.4, 10.7 Hz) |
| A16 | 2-[4-[2-[(2R)-2- | 463.39 | m/z 464.0 | 400 MHz, METHANOL-d4 δ |
| | (difluoromethyl)azetidin-1-yl]- | | (M + H)⁺, RT: | 8.40 (d, J = 1.63 Hz, 1H) |
| | 5-fluoro-6- | | 2.83 min | 8.12-8.22 (m, 1H), 6.15- |
| | (trifluoromethyl)pyrimidin-4- | | (Method F) | 6.51 (m, 1H), 5.26 (s, 2H), |
| | yl]pyrazol-1-yl]-1-piperazin-1- | | | 4.60-4.73 (m, 1H), 4.03- |
| | yl-ethanone | | | 4.16 (m, 2H), 3.58 (br s, |
| | | | | 4H), 2.80-2.92 (m, 4H), |
| | | | | 2.40-2.61 (m, 2H) |
| A15 | 2-[4-[5-cyclopropyl-6- | 475.18 | m/z 476.3 | 400 MHz, METHANOL-d4 δ |
| | (difluoromethyl)-2-[(2R)-2- | | (M + H)⁺, RT: | ppm 2.43-2.70 (m, 2 H) |
| | (difluoromethyl)azetidin-1- | | 2.072 min | 3.22 (br dd, J = 4.31, 1.94 |
| | yl]pyrimidin-4-yl]pyrazol-1-yl]- | | (Method F) | Hz, 2 H) 3.32-3.40 (m, 2 |
| | 1-piperazin-1-yl-ethanone | | | H) 3.78 (s, 3 H) 3.82-3.94 |
| | | | | (m, 4 H) 4.15 (br d, J = 8.25 |
| | | | | Hz, 2 H) 4.87-4.92 (m, 1 |
| | | | | H) 5.31 (s, 2 H) 6.55-6.97 |
| | | | | (m, 1 H) 8.22 (s, 1 H) 8.39 |
| | | | | (s, 1 H) |
| A14 | (R)-2-(4-(6-(difluoromethyl)- | 427.41 | m/z 428.2 | 1H NMR (400 MHz, |
| | 2-(2-(difluoromethyl)azetidin- | | (M + H)⁺, RT: | METHANOL-d4) 0 ppm |
| | 1-yl)pyrimidin-4-yl)-1H- | | 1.951 min | 2.38-2.50 (m, 1 H) 2.57 |
| | pyrazol-1-yl)-1-(piperazin-1- | | (Method F) | (td, J = 6.05, 2.69 Hz, 1 H) |
| | yl)ethan-1-one | | | 3.26 (br s, 2 H) 3.34 (br s, 2 |
| | | | | H) 3.87 (br s, 4 H) 4.04- |
| | | | | 4.19 (m, 2 H) 4.62-4.77 |
| | | | | (m, 1 H) 5.27 (s, 2 H) 6.47 |
| | | | | (m, 2 H) 7.18 (s, 1 H) 8.16 |
| | | | | (s, 1 H) 8.34 (s, 1 H) |
| A13 | (R)-2-(4-(6-(difluoromethyl)- | 467.47 | m/z 468.3 | 400 MHz, METHANOL-d4 δ |
| | 5-methoxy-2-(2- | | (M + H)⁺, RT: | ppm 0.33-0.49 (m, 2 H) |
| | (trifluoromethyl)azetidin-1- | | 2.07 min | 1.07-1.20 (m, 2 H) 1.89- |
| | yl)pyrimidin-4-yl)-1H-pyrazol- | | (Method F) | 2.01 (m, 1 H) 2.36-2.47 |
| | 1-yl)-1-(piperazin-1-yl)ethan- | | | (m, 1 H) 2.50-2.61 (m, 1 |
| | 1-one | | | H) 3.27 (br s, 2 H) 3.33- |
| | | | | 3.43 (m, 2 H) 3.81-3.94 |
| | | | | (m, 4 H) 4.03-4.14 (m, 2 |
| | | | | H) 4.62-4.70 (m, 1 H) 5.19- |
| | | | | 5.36 (m, 2 H) 6.17-6.51 |
| | | | | (m, 1 H) 6.97-7.30 (m, 1 |
| | | | | H) 8.18 (s, 1 H) 8.35 (s, 1 H) |
| A32 | 2-[4-[5-fluoro-2-[(2S)-2- | 427.41 | m/z 428.0 | 400 MHz, METHANOL-d4 δ |
| | methylazetidin-1-yl]-6- | | (M + H)⁺, RT: | ppm 8.35 (s, 1 H) 8.13 (s, 1 |
| | (trifluoromethyl)pyrimidin-4- | | 2.875 min | H) 5.25 (s, 2 H) 4.40-4.60 |
| | yl]pyrazol-1-yl]-1-piperazin-1- | | (Method F) | (m, 1 H) 3.92-4.14 (m, 2 |
| | yl-ethanone | | | H) 3.57 (br s, 4 H) 2.74- |
| | | | | 3.01 (m, 4 H) 2.39-2.58 |
| | | | | (m, 1 H) 1.93-2.13 (m, 1 |
| | | | | H) 1.54 (d, J = 6.11 Hz, 3 H) |
| A30 | 2-[4-[5-cyclopropyl-2-[(2S)-2- | 449.48 | m/z 450.2 | METHANOL-d4, 400 MHz δ |
| | methylazetidin-1-yl]-6- | | (M + H)⁺, RT: | 8.31 (s, 1H), 8.14 (s, 1H), |
| | (trifluoromethyl) pyrimidin-4- | | 0.345 min | 5.28 (s, 2H), 4.5-4.6 (m, |
| | yl]pyrazol-1-yl]-1-piperazin-1- | | (Method E) | 1H), 3.9-4.1 (m, 2H), 3.87 |
| | yl-ethanone | | | (br s, 4H), 3.35 (br s, 2H), |
| | | | | 3.27 (br s, 2H), 2.47 (dtd, |
| | | | | 1H, J = 5.0, 8.6, 10.9 Hz), |
| | | | | 1.9-2.1 (m, 2H), 1.53 (d, |
| | | | | 3H, J = 6.3 Hz), 1.0-1.1 (m, |
| | | | | 2H), 0.3-0.4 (m, 2H) |
| A26 | 2-[4-[6-(difluoromethyl)-5- | 419.48 | m/z 420.1 | 400 MHz, METHANOL-d4 |
| | ethyl-2-[(2S)-2- | | (M + H)⁺, RT: | 08.18-8.27 (m, 1H) 8.07 (s, |
| | methylazetidin-1-yl] | | 2.707 min | 1H), 6.45-6.77 (m, 1H), |
| | pyrimidin-4-yl] pyrazol-1-yl]- | | (Method F) | 5.24 (s, 2H), 4.44-4.55 (m, |
| | 1-piperazin-1-yl-ethanone | | | 1H), 3.93-4.11 (m, 2H), |
| | | | | 3.56-3.64 (m, 4H), 2.90 (br |
| | | | | dd, J = 15.41, 7.58 Hz, 6H), |
| | | | | 2.45 (dtd, J = 10.79, 8.54, |
| | | | | 4.77 Hz, 1H), 1.95-2.08 (m, |
| | | | | 1H), 1.54 (d, J = 6.24 Hz, |
| | | | | 3H), 1.20 (t, J = 7.34 Hz, |
| | | | | 3H), |
| A23 | (S)-2-(4-(6-(difluoromethyl)- | 421.45 | m/z 422.3 | 400 MHz, METHANOL-d4 δ |
| | 5-methoxy-2-(2- | | (M + H)⁺, RT: | ppm 8.38 (s, 1 H) 8.19 (s, 1 |
| | methylazetidin-1-yl)pyrimidin- | | 1.994 min | H) 6.40-6.95 (t, 1 H) 5.30 |
| | 4-yl)-1H-pyrazol-1-yl)-1- | | (Method E) | (s, 2 H) 4.40-4.57 (m, 1 H) |
| | (piperazin-1-yl)ethan-1-one | | | 4.05 (br s, 1 H) 3.98 (br d, J = |
| | | | | 8.13 Hz, 1 H) 3.87 (br s, 4 |
| | | | | H) 3.74 (s, 3 H) 3.35 (br s, 2 |
| | | | | H) 3.27 (br s, 2 H) 2.33- |
| | | | | 2.54 (m, 1 H) 2.03 (br d, |
| | | | | J = 7.50 Hz, 1 H) 1.54 (br d, |
| | | | | J = 6.00 Hz, 3 H) |
| A22 | 2-[4-[6-(difluoromethyl)-5- | 409.42 | m/z 410.0 | 400 MHz, METHANOL-d4 δ |
| | fluoro-2-[(2S)-2- | | (M + H)⁺, RT: | ppm 8.33 (d, J = 1.67 Hz, 1 |
| | methylazetidin-1-yl]pyrimidin- | | 2.641 min | H) 8.15 (s, 1 H) 6.68 (t, |
| | 4-yl]pyrazol-1-yl]-1-piperazin- | | (Method F) | J = 53.47 Hz, 1 H) 5.31 (s, 2 |
| | 1-yl-ethanone | | | H) 4.45-4.55 (m, 1 H) 4.01 |
| | | | | (br d, J = 8.94 Hz, 2 H) 3.87 |
| | | | | (br s, 4 H) 3.35 (br s, 2 H) |
| | | | | 3.26 (br s, 2 H) 2.46 (ddd, |
| | | | | J = 8.37, 4.86, 2.27 Hz, 1 H) |
| | | | | 1.96-2.09 (m, 1 H) 1.54 (d, |
| | | | | J = 6.32 Hz, 3 H) |
| A34 | (2S,3R)-1-[5-cyclopropyl-4- | 408.43 | m/z 409.3 | 400 MHz, METHANOL-d4 δ |
| | [1-(1-methylazetidin-3- | | (M + H)⁺, RT: | ppm 8.36 (s, 1 H) 8.31 (s, 1 |
| | yl)pyrazol-4-yl]-6- | | 2.076 min | H) 5.41-5.50 (m, 1 H) 4.66- |
| | (trifluoromethyl)pyrimidin-2- | | (Method E) | 4.79 (m, 2 H) 4.61 (br d, |
| | yl]-2-methyl-azetidin-3-ol | | | J = 3.50 Hz, 2 H) 4.27 (dd, |
| | | | | J = 9.13, 5.88 Hz, 1 H) 4.11- |
| | | | | 4.22 (m, 2 H) 3.73 (dd, J = |
| | | | | 9.19, 4.69 Hz, 1 H) 3.11 (br |
| | | | | s, 3 H) 1.95-2.07 (m, 1 H) |
| | | | | 1.53 (d, J = 6.13 Hz, 3 H) |
| | | | | 1.05 (br d, J = 8.25 Hz, 2 H) |
| | | | | 0.24-0.44 (m, 2 H) |
| A27 | (2S, 3R)-1-[5-fluoro-4-[1-(1- | 386.35 | m/z 387.1 | 400 MHz, METHANOL-d4 |
| | methylazetidin-3-yl) pyrazol- | | (M + H)⁺, RT: | δ 8.44 (d, J = 1.47 Hz, 1H) |
| | 4-yl]-6-(trifluoromethyl) | | 2.735 min | 8.19 (s, 1H), 5.13 (br t, |
| | pyrimidin-2-yl]-2-methyl- | | (Method F) | J = 6.85 Hz, 1H), 4.29 (dd, |
| | azetidin-3-ol | | | J = 9.17, 5.87 Hz, 1H), 4.12- |
| | | | | 4.22 (m, 2H), 3.87 (br s, |
| | | | | 2H), 3.76 (dd, J = 9.23, 4.58 |
| | | | | Hz, 1H), 3.59-3.73 (m, 2H), |
| | | | | 2.48 (br s, 3H), 1.55 (d, |
| | | | | J = 5.99 Hz, 3H) |
| A19 | (2S,3R)-1-(4- | 380.40 | m/z 381.3 | 400 MHz, METHANOL-d4 δ |
| | (difluoromethyl)-5-methoxy- | | (M + H)⁺, RT: | ppm 8.44 (s, 1 H) 8.37 (s, 1 |
| | 6-(1-(1-methylazetidin-3-yl)- | | 1.757 min | H) 6.76 (t, J = 53.91 Hz, 1 |
| | 1H-pyrazol-4-yl)pyrimidin-2- | | (Method E) | H) 5.39-5.56 (m, 1 H) 4.48- |
| | yl)-2-methylazetidin-3-ol | | | 4.76 (m, 4 H) 4.24-4.36 |
| | | | | (m, 1 H) 4.03-4.22 (m, 2 |
| | | | | H) 3.74 (s, 3 H) 3.71 (br s, 1 |
| | | | | H) 3.12 (br s, 3 H) 1.55 (br |
| | | | | d, J = 6.00 Hz, 3 H) |

### Testing of Activity of Compounds

The utility of the compounds of the present disclosure and the salts of such compounds as medical agents in the treatment of the diseases/conditions described herein in mammals (e.g. humans, male or female) is demonstrated by the activity and advantages of the compounds of the present disclosure in one or more of the conventional assays and in vivo assays described and noted herein. The in vivo assays (with appropriate modifications within the skill in the art) can be used to determine the activity of other agents as well as the compounds of the present disclosure. Thus, the protocols described herein can also be used to demonstrate the utility of the combinations of the compounds of the present disclosure. The assays and models may also demonstrate particular other property advantages e.g., side effect profile; half-life. In addition, such assays provide a means whereby the activities of the compounds of the present disclosure and the salts of such compounds (or the other agents described herein) can be compared to each other and with the activities of other known compounds. The results of these comparisons are useful for determining dosage levels in mammals, including humans, far the treatment of such diseases.

Absorption, Distribution, Metabolism and Excretion (ADME) and pharmacokinetics (PK) of compounds and exemplary assays are discussed in the on-line publication by Thomas D. Y. Chung, David B. Terry and Layton H. Smith “In Vitro and In Vivo Assessment of ADME and PK Properties During Lead Selection and Lead Optimization-Guidelines, Benchmarks and Rules of Thumb—(https://www.ncbi.nlm.nih.gov/books/NBK326710/).

Animal models of nonalcoholic fatty liver disease are described in Nutrients, 2017 Oct. 9(10): 1072; Int. J. Mol. Sci. 2022, 23, 15791 and Digestion 2020; 101:522-535. Animal models of fructose inhibition are described in Molecular Metabolism, 2021; 48:101196.

### Example 10: KHK Inhibition Assays

Determination of Human and Rat recombinant KHK-A and KHK-C isozyme IC₅₀ Values

An assay reagent cocktail was prepared by combining NADH, water, TEA, KCl, MgCl₂, PEP, ATP, DTT, coupling enzymes (pyruvate kinase and lactate dehydrogenase, LDH) to final concentrations as shown in Table B.

TABLE B

| Reagent | Final Concentration | |
|---|---|---|
| NADH | 300 | μM |
| Water | — | |
| TEA | 33 | mM |
| KCl | 100 | mM |
| MgCl₂ | 6 | mM |
| PEP | 1.33 | mM |
| ATP | 0.1 | mM |
| DTT | 12 | mM |
| Pyruvate kinase | 1.0 | U/mL |
| LDH | 1.0 | U/mL |

To this was added the relevant KHK isozyme to a final concentration of 6 nM. Aliquots of each inhibitor compound were diluted via 5-fold serial dilutions to produce final concentrations ranging from 1000 nM to 0.064 nM. The inhibitor aliquots were added to the assay reagent cocktail containing KHK with fructose (at a concentration of 2 mM) in a 96-well plate. The absorbance at 340 nm was measured via spectrophotometry and inhibition was analyzed using non-linear regression.

Results of the assay are presented in Table C.

TABLE C

| | Human | Human | Rat | Rat |
|---|---|---|---|---|
| | KHK-C IC50 | KHK-A IC50 | KHK-C IC50 | KHK-A IC50 |
| Compound | (nM) | (nM) | (nM) | (nM) |
| A1 | 4.06 | 6 | 9 | 2.2 |
| A2 | 31 | 13.5 | 26 | 9 |
| A3 | 7.48 | 4.6 | 8.3 | 2.9 |
| A4 | 5.94 | 1.8 | 4.9 | 1.3 |
| A5 | 30.6 | 69 | 44 | 26 |
| A6 | 15.7 | 8.8 | 11 | 2.3 |
| A7 | 231 | 160 | 120 | 46 |
| A8 | 6.46 | 7.8 | 16 | 5.5 |
| A9 | 40.5 | 22 | 37 | 10 |
| A10 | 0.856 | 0.2 | 1.1 | 0.2 |
| A11 | 2.09 | 8.5 | 2.1 | 0.96 |
| A12 | 1.05 | 4.1 | 3.3 | 1.3 |
| A13 | 41.1 | not | N/A | N/A |
| | | available | | |
| | | (N/A) | | |
| A14 | 318 | N/A | N/A | N/A |
| A15 | 32.7 | N/A | N/A | N/A |
| A16 | 47.8 | N/A | N/A | N/A |
| A17 | 74.9 | N/A | N/A | N/A |
| A18 | 7.42 | 5.3 | 9.5 | 4.1 |
| A19 | 10.6 | 27 | 17 | 6.6 |
| A20 | 133 | N/A | N/A | N/A |
| A21 | 1.5 | 2.05 | 3.33 | 1.46 |
| A22 | 65.8 | N/A | N/A | N/A |
| A23 | 54.6 | N/A | N/A | N/A |
| A24 | 14.7 | 31 | 17 | 5.2 |
| A25 | 5.22 | 2.2 | 15 | 5.7 |
| A26 | 32.9 | 13 | 26 | 5.2 |
| A27 | 4.09 | N/A | N/A | N/A |
| A28 | 18 | 15 | 20 | 4.1 |
| A29 | 5.03 | N/A | N/A | N/A |
| A30 | 41.3 | N/A | N/A | N/A |
| A31 | 16.8 | N/A | N/A | N/A |
| A32 | 23.2 | 12 | 20 | 8.2 |
| A33 | 90.9 | 65 | 86 | 23 |
| A34 | 25.4 | N/A | N/A | N/A |
| A35 | 4.85 | 1.9 | 7.7 | 4.1 |
| A36 | 36.6 | N/A | N/A | N/A |
| A37 | 11.9 | N/A | N/A | N/A |
| A38 | 3.64 | 6.5 | 14 | 7.3 |
| A39 | 110 | N/A | N/A | N/A |
| A40 | 33.2 | N/A | N/A | N/A |
| A41 | 17.1 | N/A | N/A | N/A |
| A42 | 18.3 | 13 | 26 | 10 |
| A43 | 8.03 | 4.5 | 12 | 6.8 |
| A44 | 61 | N/A | N/A | N/A |

### Example 11: Cell-Based Potency Assay (HepG2 Assay)

Testing inhibition effect of compounds against human KHK in HepG2 Cells

HepG2 cells were grown in RPMI1640 supplemented media on 100 mm plates to near confluency (about 5-8×10⁶ cells). The growth media was aspirated and 4 mL trypsin solution (0.25% (w/v)+0.25% (w/v) EDTA) was added, followed by incubation at 37° C. for 5-10 mins. The cells were pelleted and resuspended in 2 mL complete media.

The cells were then placed in a 96 well plate at 20,000-50,000 cells per well and grown for 20-28 hours at 37° C. to allow adherence.

Inhibitor compounds were diluted in DMSO to 50 μM, then serially diluted with MEM media (ThermoFisher). Final inhibitor concentration ranged from 1000 nM to 0.064 nM (5-fold dilution series).

The RPMI medium was removed from the plate of confluent cells. The plate was then incubated for 35 min at 37°, after which were added 0.1 mL of the diluted inhibitor compounds in MEM with fructose, 0.1 mL of MEM media without inhibitor compound or fructose, or trypsin; yielding a final concentration of fructose at 20 mM. The plate was subsequently incubated for 20 min at 37°, then placed on ice to stop the reactions.

Next, 200 μL of cold 80% (v/v) methanol:water was added to all wells, except the controls used to enumerate cell count. The plate was vortexed and cell debris removed by centrifugation. To measure F1P produced, the resulting supernatant fraction was analyzed by LC/MS.

IC50 data is presented in Table D.

TABLE D

| Compound | HepG2 IC50 (nM) | |
|---|---|---|
| C1 | 160 | (n = 1) |
| C2 | 91 | (n = 32) |
| C3 | 4 | (n = 2) |
| A1 | 7 | (n = 1) |
| A2 | 48 | (n = 2) |
| A3 | 25 | (n = 2) |
| A6 | 70 | (n = 1) |
| A7 | 332 | (n = 1) |
| A11 | 4 | (n = 1) |
| A12 | 1 | (n = 2) |
| A18 | 11 | (n = 1) |
| A19 | 23 | (n = 1) |
| A21 | 2 | (n = 3) |
| A24 | 28 | (n = 2) |
| A27 | 9 | (n = 1) |
| A35 | 9 | (n = 1) |
| A38 | 36 | (n = 1) |
| A42 | 43 | (n = 1) |
| A43 | 23 | (n = 1) |

The structures of compounds C₁-C₃ are provided in paragraph [00374].

### Example 12: Cell-Based Potency Assay (KHK-C-Overexpressing HepG2 Assay)

Testing inhibition effect of compounds against human KHK-C in engineered HepG2 cells.

Engineered HepG2 cells were generated using methods known to those of skill in the art. Briefly, HepG2 cells were incubated with lentivirus carrying a transgene encoding human KHK-C, and grown under antibiotic selection.

KHK-C-overexpressing HepG2 cells were grown in RPMI1640 supplemented media on T-182 cm² flask to near confluency (about 25-40×10⁶ cells). The growth media was aspirated, the flask was washed with 10 mL PBS, and 4 mL trypsin solution (0.25% (w/v)+0.25% (w/v) EDTA) was added, followed by incubation at 37° C. for 3-5 mins. The cells were pelleted and resuspended in 2 mL complete media.

The cells were then placed in a 96-well plate at 20,000-40,000 cells per well and grown for 20-28 hours at 37° C. to allow adherence.

Inhibitor compounds were diluted in DMSO to 50 μM, then serially diluted with MEM media (ThermoFisher). Final inhibitor concentration ranged from 5000 nM to 0.32 nM. Control compounds C1, C2, and C3 were tested for comparison with compounds disclosed herein.

| Control Compound | Structure |
|---|---|
| C1 (also called PF-06835919, WO2017/115205 Ex. 49) | 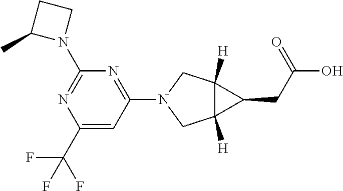 [SMILES: C[C@H]1CCN1c1nc(N2C[C@@H]3[C@@H](CC(=O)O)[C@@H]3C2)cc(C(F)(F)F)n1] |
| C2 (also called LY3522348, WO2020/257171 Ex. 1) | 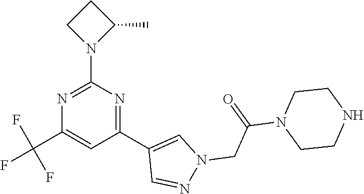 [SMILES: C[C@H]1CCN1c1nc(-c2cnn(CC(=O)N3CCNCC3)c2)cc(C(F)(F)F)n1] |
| C3 (WO2020/257171-Ex. 35) | 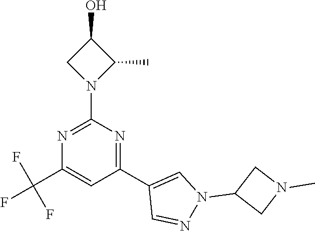 [SMILES: C[C@H]1[C@H](O)CN1c1nc(-c2cnn(C3CN(C)C3)c2)cc(C(F)(F)F)n1] |

The RPMI medium was removed from the plate of confluent cells. The plate was then incubated for 35 min at 37° C. with 0.1 mL of the diluted inhibitor compounds in MEM. After, an additional 0.1 mL of the diluted inhibitor compounds in MEM with fructose, or 0.1 mL of MEM media without inhibitor compound and/or fructose; yielding a final concentration of fructose at 30 mM was added. The plate was subsequently incubated for 5 min at 37° C., then placed on ice to stop the reactions.

Next 200 μL of cold 80% (v/v) methanol:water was added to al wells and placed in the −80° C. freezer for 1 hour with an adhesive seal. The plate was vortexed and cell debris removed by centrifugation. To measure F1P produced, the resulting supernatant fraction was analyzed by LC/MS.

IC50 data is presented in Table E.

TABLE E

| | KHK-C | |
|---|---|---|
| | overexpressing | |
| | HepG2, geomean | |
| Compound | IC50 (nM) | |
| C1 | 536 | (n = 5) |
| C2 | 457 | (n = 26) |
| C3 | 191 | (n = 4) |
| A3 | 186 | (n = 1) |
| A9 | 184 | (n = 1) |
| A12 | 33.5 | (n = 4) |
| A21 | 36.5 | (n = 7) |
| A35 | 82 | (n = 1) |

### Example 13: CYP Inhibition Assay in Human Liver Microsomes

### Cytochrome P450 Inhibition Profiling

A potential limitation of novel drugs for metabolic diseases is the risk of drug-drug interactions mediated by inhibition of the cytochrome P450 (CYP) enzymes that contribute to xenobiotic metabolism, including CYP1A2, CYP2C9, CYP2C8, CYP2D6, and CYP3A4, among others. Frequent concomitant medications in patients suffering from metabolic disease, type 2 diabetes, obesity, hypertension, and/or MASLD/MASH include a number of substrates, inhibitors, or inducers of the CYP enzymes, including HMG-CoA reductase inhibitors (statins), thiazolidinediones, fibrates, sulfonylureas, selective serotonin reuptake inhibitors, angiotensin II receptor blockers. Total exposure of these drugs may be affected if they are coadministered with an inhibitor of CYP enzymes. Therefore, lack of meaningful inhibition of CYP enzymes, alongside maintained potency as an inhibitor of KHK-C and KHK-A, is a desirable characteristic for a clinical candidate. Preferred compounds of this discourse have beneficial properties i.e., lacking significant CYP inhibition.

### Testing Inhibition of Compounds Against CYP Enzymes

Human liver microsomes (HLMs) were incubated with known substrates of specific CYP enzymes (for example, phenacetin for CYP1A2; diclofenac for CYP2C9; S-mephenytoin for CYP2C19; dextromethorphan for CYP2D6; midazolam for CYP3A4). Substrate metabolism to the known metabolite was monitored by LC-MS-MS. Positive control inhibitors were tested at a single concentration, and test compounds were tested in a 7-point dose-response curve starting from a high concentration of 50 μM with ˜3-fold dilution series to a low concentration of 50 nM.

Each assay well contained 0.2 mg/mL HLMs, 1 mM NADPH, substrate-dependent final concentration of each substrate, and specified concentration of either positive control inhibitor or test compound. Wells were incubated for 10 minutes at 37° C. and reactions were stopped by adding cold stop solution (for example, 200 ng/mL tolbutamide in acetonitrile) followed by centrifugation at 4000 rpm for 20 minutes to precipitate protein. Supernatant was added to 0.5-volume of HPLC water, shaken for 10 minutes, and analyzed by LC-MS-MS.

### Example 14: hERG Inhibition Assay by Automated Patch Clamp Method

An additional potential limitation of novel drugs is the risk of cardiotoxicity mediated by binding to or inhibition of cardiac ion channels, including for example hERG (human ether-a-go-go-related gene). hERG is a subunit of a potassium channel that mediates cardiac repolarization, and is inhibited by diverse classes of small molecules, which may lead to arrhythmias. Therefore, maximizing the window between potency as an inhibitor of the target enzyme, ketohexokinase, and the potency as an inhibitor of hERG is a desirable characteristic for a clinical candidate. Preferred compounds of this disclosure have beneficial properties i.e., lacking significant hERG inhibition and/or a large window between KHK inhibitor potency and hERG inhibitor potency.

Testing Inhibition of Compounds Against hERG (Human Ether-Ago-go-Related Gene)

CHO cells stably expressing hERG channels were seeded in ˜298 mOsm 10 mM HEPES buffer, pH 7.4 containing 140 mM NaCl, 4 mM KCl, 2 mM CaCl₂, 5 mM glucose on the Nanion SyncroPatch 384PE for profiling using the automated patch clamp method.

Positive control (amitriptyline) and test compounds were tested in a 5-point dose-response curve starting from a high concentration of 30 μM with 3-fold dilution series to a low concentration of 300 nM.

Currents were elicited using a voltage command protocol consisting of a continuous holding potential of −80 mV, first stepped to −50 mM for 80 msec for leak subtraction, then stepped to +20 mV for 4,800 msec to open hERG channels, then stepped to −50 mV for 5,000 msec causing a hERG “tail current” that was measured and collected for analysis, then stepped to the holding potential of −80 mV for 1,000 msec. This command protocol was repeated every 20 seconds, continuously during the assay (300 seconds before addition of control or test compound, and 300 seconds following addition of control or test compound).

### Example 15: Steady-State Kinetics

An additional potential limitation of novel drugs inhibiting ketohexokinase isoforms is the risk that increasing concentrations of substrate (for example, fructose and/or ATP) diminish the potency of the drugs due to a competitive mode of inhibition. Inhibition of ketohexokinase metabolism of fructose to fructose-1-phosphate is expected to increase the concentration of both ATP and fructose, potentially to an extent that may outcompete the novel drug as a competitive inhibitor. Therefore, noncompetitive modes of inhibition of the target enzyme, i.e., where potency as an enzyme inhibitor is not affected by the concentration of any enzyme substrate, may be a desirable characteristic for a clinical candidate. Mode of inhibition may be determined through a series of steady state kinetics experiments, performed as known by one skilled in the art, for example as described in Copeland, RA, Evaluation of Enzyme Inhibitors in Drug Discovery: A Guide for Medicinal Chemists and Pharmacologists (2013), where compound potency as an inhibitor of ketohexokinase is determined across a range of substrate concentrations, i.e., varying concentration of ATP in excess of fructose; or varying concentration of fructose in excess of ATP. Preferred compounds of this disclosure have beneficial properties with respect to the mode of inhibition, whereby increased ATP and/or fructose concentrations do not impact compound potency as an inhibitor of ketohexokinase.

All references provided herein are incorporated herein in their entirety by reference. As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997.

It is to be understood that while the disclosure has been described in conjunction with the detailed description thereof, the foregoing description is intended to illustrate and not limit the scope of the discourse, which is defined by the scope of the appended claims. Other aspects, advantages, and modifications are within the scope of the following claims.

1. A compound having a structure of Formula I, or a pharmaceutically acceptable salt thereof: 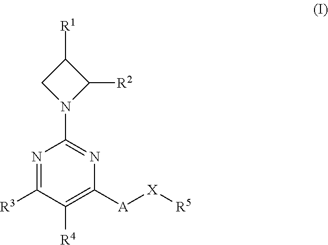
wherein
R¹ is H or OH;
R² is C₁₋₆alkyl or C₁₋₆haloalkyl;
R³ is C₁₋₆alkyl or C₁₋₆haloalkyl;
R⁴ is H, halo, CN, C₁₋₆alkyl, C₁₋₆alkoxy, or C₃₋₅cycloalkyl;
A is a 5-membered heteroaryl comprising 2-3 nitrogen ring atoms;
X is a bond or C₁₋₆alkylene-C(O); and
R⁵ is 4- to 6-membered heterocycloalkyl having 1 or 2 ring nitrogen atoms, wherein the heterocycloalkyl is optionally substituted with 1 or 2 C₁₋₆alkyl;
with the proviso that when R⁴ is H, compounds where both R² is methyl and R³ is trifluoromethyl are excluded.

2. The compound or salt of claim 1, wherein A is pyrazolyl.

3. The compound or salt of claim 1, having the structure of Formula Ix:

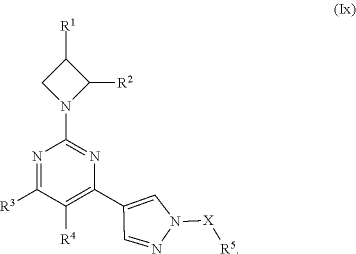

4. The compound or salt of claim 1, having the structure of Formula II: 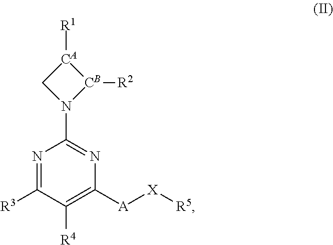
wherein C^{A} and C^{B} represent carbon stereocenters having the same or opposite stereochemistry.

5. The compound or salt of claim 4, wherein C^{A} is a carbon in the R configuration and C^{B} is a carbon in the S configuration.

6. The compound or salt of claim 4, wherein A is pyrazolyl.

7. (canceled)

8. (canceled)

9. The compound or salt of claim 1, wherein;
(i) R⁵ is 4-membered heterocycloalkyl having 1 ring nitrogen atom, wherein the heterocycloalkyl is optionally substituted with 1 or 2 C₁₋₆alkyl; or
(ii) R₅ is 6-membered heterocycloalkyl having 2 ring nitrogen atoms, wherein the heterocycloalkyl is optionally substituted with 1 or 2 C₁₋₆alkyl.

10. (canceled)

11. The compound or salt of claim 9, wherein R⁵ is unsubstituted.

12. The compound or salt of claim 9, wherein R⁵ is substituted with 1 C₁₋₆alkyl.

13. (canceled)

14. (canceled)

15. (canceled)

16. (canceled)

17. The compound or salt of claim 1, wherein R² is methyl.

18. (canceled)

19. The compound or salt of claim 1, wherein R² is CHF₂ or CF₃.

20. The compound or salt of claim 1, wherein R³ is CHF₂ or CF₃.

21. (canceled)

22. The compound or salt of claim 1, wherein R⁴ is H, F, Cl, methoxy, methyl, ethyl, or cyclopropyl.

23. (canceled)

24. (canceled)

25. (canceled)

26. (canceled)

27. (canceled)

28. (canceled)

29. (canceled)

30. The compound or salt of claim 1, wherein R³ is CHF₂ and R⁴ is methyl.

31. (canceled)

32. (canceled)

33. The compound of claim 5 wherein R¹ is H or hydroxy, R² is methyl, R³ is CHF₂, R⁴ is methyl, X is a bond and R⁵ is a 4- to 6-membered heterocycloalkyl having 1 ring nitrogen atom, wherein the heterocycloalkyl is optionally substituted with C₁₋₂alkyl.

34. The compound of claim 5 wherein X is C₁₋₆alkylene-C(O), R⁵ is azetidinyl optionally substituted with 1 or 2 C₁₋₆alkyl, R¹ is H or hydroxy, R² is C₁₋₃alkyl, R³ is CHF₂, and R⁴ is methyl.

35. A compound as recited in Table A, or a pharmaceutically acceptable salt thereof;
TABLE A

| | Name | Structure |
|---|---|---|
| A1 | 2-(4-{2-[(R)-2- (trifluoromethyl)-1- azetidinyl]-5-methyl-6- (trifluoromethyl)-4- pyrimidinyl}-1-pyrazolyl)- 1-(1-piperazinyl)-1- ethanone | 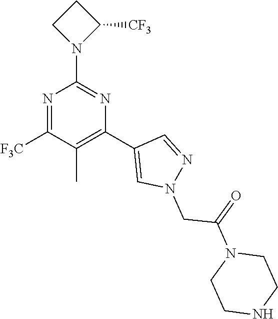 [SMILES: Cc1c(-c2cnn(CC(=O)N3CCNCC3)c2)nc(N2CC[C@@H]2C(F)(F)F)nc1C(F)(F)F] |
| A2 | 2-(4-{2-[(R)-2- (trifluoromethyl)-1- azetidinyl]-6- (trifluoromethyl)-4- pyrimidinyl}-1-pyrazolyl)- 1-(1-piperazinyl)-1- ethanone | 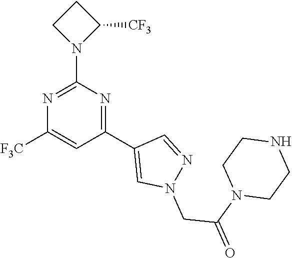 [SMILES: O=C(Cn1cc(-c2cc(C(F)(F)F)nc(N3CC[C@@H]3C(F)(F)F)n2)cn1)N1CCNCC1] |
| A3 | 2-(4-{2-[(S)-2-methyl-1- azetidinyl]-5-methyl-6- (trifluoromethyl)-4- pyrimidinyl}-1-pyrazolyl)- 1-(1-piperazinyl)-1- ethanone | 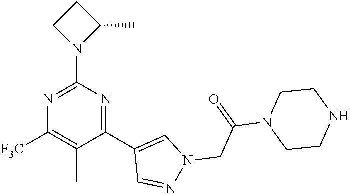 [SMILES: Cc1c(-c2cnn(CC(=O)N3CCNCC3)c2)nc(N2CC[C@@H]2C)nc1C(F)(F)F] |
| A4 | 2-(4-{2-[(S)-2-methyl-1- azetidinyl]-5-chloro-6- (trifluoromethyl)-4- pyrimidinyl}-1-pyrazolyl)- 1-(1-piperazinyl)-1- ethanone | 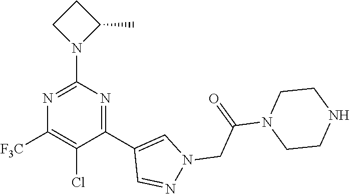 [SMILES: C[C@H]1CCN1c1nc(-c2cnn(CC(=O)N3CCNCC3)c2)c(Cl)c(C(F)(F)F)n1] |
| A5 | 2-(4-{2-[(S)-2-methyl-1- azetidinyl]-5-methoxy-6- (trifluoromethyl)-4- pyrimidinyl}-1-pyrazolyl)- 1-(1-piperazinyl)-1- ethanone | 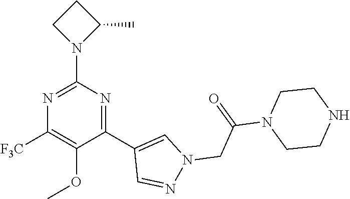 [SMILES: COc1c(-c2cnn(CC(=O)N3CCNCC3)c2)nc(N2CC[C@@H]2C)nc1C(F)(F)F] |
| A6 | 2-(4-{2-[(S)-2-methyl-1- azetidinyl]-5-ethyl-6- (trifluoromethyl)-4- pyrimidinyl}-1-pyrazolyl)- 1-(1-piperazinyl)-1- ethanone | 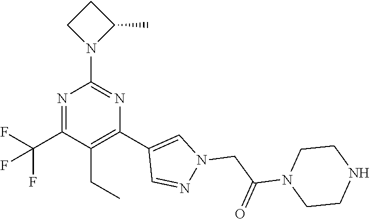 [SMILES: CCc1c(-c2cnn(CC(=O)N3CCNCC3)c2)nc(N2CC[C@@H]2C)nc1C(F)(F)F] |
| A7 | 2-(4-{2-[(S)-2-methyl-1- azetidinyl]-6- (difluoromethyl)-4- pyrimidinyl}-1-pyrazolyl)- 1-(1-piperazinyl)-1- ethanone | 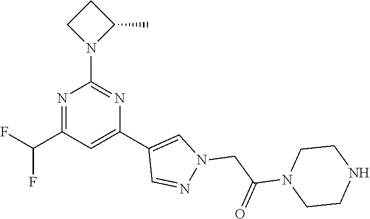 [SMILES: C[C@H]1CCN1c1nc(-c2cnn(CC(=O)N3CCNCC3)c2)cc(C(F)F)n1] |
| A8 | (2S,3R)-1-{5-methoxy-4- [1-(1-methyl-3-azetidinyl)- 4-pyrazolyl]-6- (trifluoromethyl)-2- pyrimidinyl}-2-methyl-3- azetidinol | 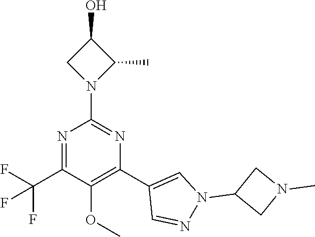 [SMILES: COc1c(-c2cnn(C3CN(C)C3)c2)nc(N2C[C@@H](O)[C@@H]2C)nc1C(F)(F)F] |
| A9 | (2S,3R)-1-{6- (difluoromethyl)-4-[1-(1- methyl-3-azetidinyl)-4- pyrazolyl]-2-pyrimidinyl}- 2-methyl-3-azetidinol | 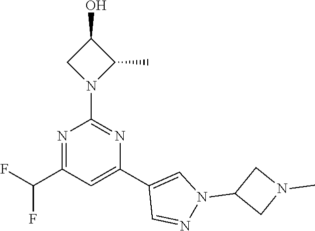 [SMILES: C[C@H]1[C@H](O)CN1c1nc(-c2cnn(C3CN(C)C3)c2)cc(C(F)F)n1] |
| A10 | (2S,3R)-1-{5-chloro-4-[1- (1-methyl-3-azetidinyl)-4- pyrazolyl]-6- (trifluoromethyl)-2- pyrimidinyl}-2-methyl-3- azetidinol | 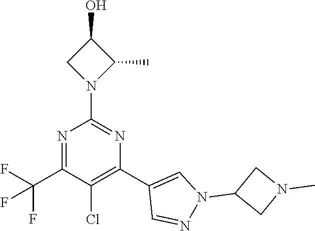 [SMILES: C[C@H]1[C@H](O)CN1c1nc(-c2cnn(C3CN(C)C3)c2)c(Cl)c(C(F)(F)F)n1] |
| A11 | (2S,3R)-1-{5-ethyl-4-[1- (1-methyl-3-azetidinyl)-4- pyrazolyl]-6- (trifluoromethyl)-2- pyrimidinyl}-2-methyl-3- azetidinol | 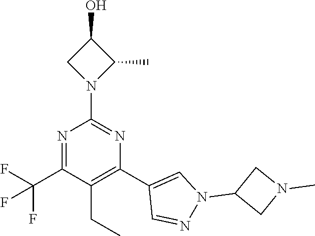 [SMILES: CCc1c(-c2cnn(C3CN(C)C3)c2)nc(N2C[C@@H](O)[C@@H]2C)nc1C(F)(F)F] |
| A12 | (2S,3R)-2-methyl-1-{5- methyl-4-[1-(1-methyl-3- azetidinyl)-4-pyrazolyl]-6- (trifluoromethyl)-2- pyrimidinyl}-3-azetidinol | 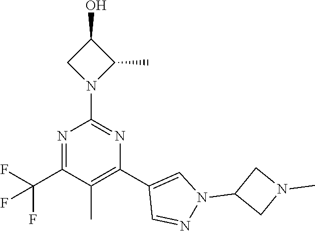 [SMILES: Cc1c(-c2cnn(C3CN(C)C3)c2)nc(N2C[C@@H](O)[C@@H]2C)nc1C(F)(F)F] |
| A13 | 2-(4-{2-[(R)-2- (difluoromethyl)-1- azetidinyl]-5-cyclopropyl- 6-(difluoromethyl)-4- pyrimidinyl}-1-pyrazolyl)- 1-(1-piperazinyl)-1- ethanone | 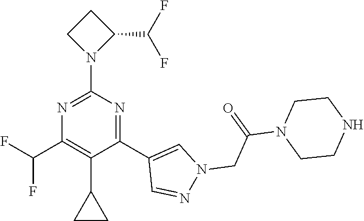 [SMILES: O=C(Cn1cc(-c2nc(N3CC[C@@H]3C(F)F)nc(C(F)F)c2C2CC2)cn1)N1CCNCC1] |
| A14 | 2-(4-{2-[(R)-2- (difluoromethyl)-1- azetidinyl]-6- (difluoromethyl)-4- pyrimidinyl}-1-pyrazolyl)- 1-(1-piperazinyl)-1- ethanone | 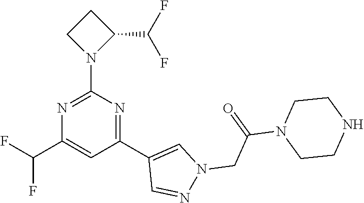 [SMILES: O=C(Cn1cc(-c2cc(C(F)F)nc(N3CC[C@@H]3C(F)F)n2)cn1)N1CCNCC1] |
| A15 | 2-(4-{2-[(R)-2- (trifluoromethyl)-1- azetidinyl]-6- (difluoromethyl)-5- methoxy-4-pyrimidinyl}- 1-pyrazolyl)-1-(1- piperazinyl)-1-ethanone | 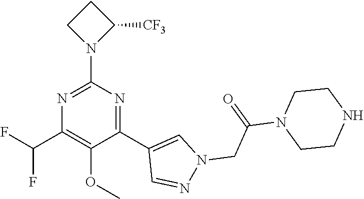 [SMILES: COc1c(-c2cnn(CC(=O)N3CCNCC3)c2)nc(N2CC[C@@H]2C(F)(F)F)nc1C(F)F] |
| A16 | 2-(4-{2-[(R)-2- (difluoromethyl)-1- azetidinyl]-5-fluoro-6- (trifluoromethyl)-4- pyrimidinyl}-1-pyrazolyl)- 1-(1-piperazinyl)-1- ethanone | 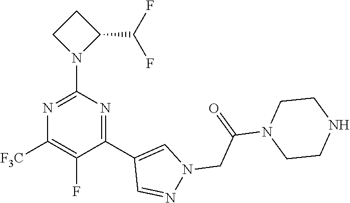 [SMILES: O=C(Cn1cc(-c2nc(N3CC[C@@H]3C(F)F)nc(C(F)(F)F)c2F)cn1)N1CCNCC1] |
| A17 | 2-(4-{2-[(R)-2- (difluoromethyl)-1- azetidinyl]-5-cyclopropyl- 6-(trifluoromethyl)-4- pyrimidinyl}-1-pyrazolyl)- 1-(1-piperazinyl)-1- ethanone | 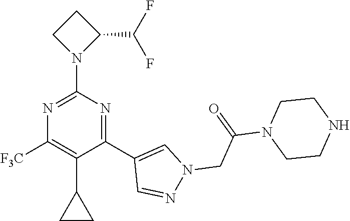 [SMILES: O=C(Cn1cc(-c2nc(N3CC[C@@H]3C(F)F)nc(C(F)(F)F)c2C2CC2)cn1)N1CCNCC1] |
| A18 | 2-(4-12-[(R)-2- (trifluoromethyl)-1- azetidinyl]-6- (difluoromethyl)-5-methyl- 4-pyrimidinyl}-1- pyrazolyl)-1-(1- piperazinyl)-1-ethanone | 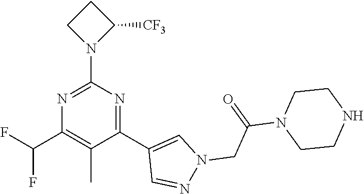 [SMILES: Cc1c(-c2cnn(CC(=O)N3CCNCC3)c2)nc(N2CC[C@@H]2C(F)(F)F)nc1C(F)F] |
| A19 | (2S,3R)-1-{6- (difluoromethyl)-5- methoxy-4-[1-(1-methyl-3- azetidinyl)-4-pyrazolyl]-2- pyrimidinyl}-2-methyl-3- azetidinol | 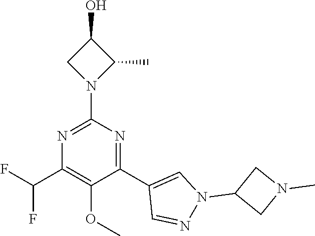 [SMILES: COc1c(-c2cnn(C3CN(C)C3)c2)nc(N2C[C@@H](O)[C@@H]2C)nc1C(F)F] |
| A20 | 2-(4-{2-[(R)-2- (trifluoromethyl)-1- azetidinyl]-6- (difluoromethyl)-4- pyrimidinyl}-1-pyrazolyl)- 1-(1-piperazinyl)-1- ethanone | 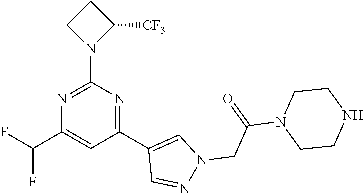 [SMILES: O=C(Cn1cc(-c2cc(C(F)F)nc(N3CC[C@@H]3C(F)(F)F)n2)cn1)N1CCNCC1] |
| A21 | (2S,3R)-1-{6- (difluoromethyl)-5-methyl- 4-[1-(1-methyl-3- azetidinyl)-4-pyrazolyl]-2- pyrimidinyl}-2-methyl-3- azetidinol | 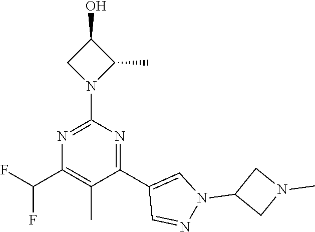 [SMILES: Cc1c(-c2cnn(C3CN(C)C3)c2)nc(N2C[C@@H](O)[C@@H]2C)nc1C(F)F] |
| A22 | 2-(4-{2-[(S)-2-methyl-1- azetidinyl]-6- (difluoromethyl)-5-fluoro- 4-pyrimidinyl}-1- pyrazolyl)-1-(1- piperazinyl)-1-ethanone | 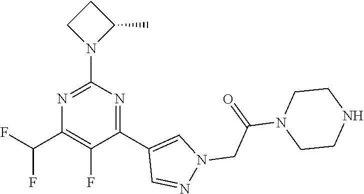 [SMILES: C[C@H]1CCN1c1nc(-c2cnn(CC(=O)N3CCNCC3)c2)c(F)c(C(F)F)n1] |
| A23 | 2-(4-{2-[(S)-2-methyl-1- azetidinyl]-6- (difluoromethyl)-5- methoxy-4-pyrimidinyl}- 1-pyrazolyl)-1-(1- piperazinyl)-1-ethanone | 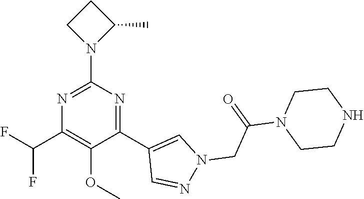 [SMILES: COc1c(-c2cnn(CC(=O)N3CCNCC3)c2)nc(N2CC[C@@H]2C)nc1C(F)F] |
| A24 | 2-(4-{2-[(S)-2-methyl-1- azetidinyl]-6- (difluoromethyl)-5-methyl- 4-pyrimidinyl}-1- pyrazolyl)-1-(1- piperazinyl)-1-ethanone | 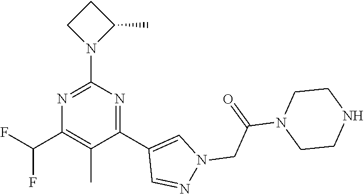 [SMILES: Cc1c(-c2cnn(CC(=O)N3CCNCC3)c2)nc(N2CC[C@@H]2C)nc1C(F)F] |
| A25 | (2R,3R)-1-{4-[1-(1- methyl-3-azetidinyl)-4- pyrazolyl]-6- (trifluoromethyl)-2- pyrimidinyl}-2- (trifluoromethyl)-3- azetidinol | 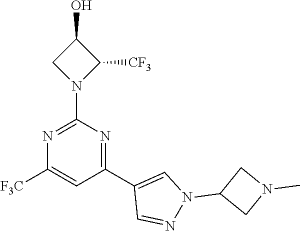 [SMILES: CN1CC(n2cc(-c3cc(C(F)(F)F)nc(N4C[C@@H](O)[C@@H]4C(F)(F)F)n3)cn2)C1] |
| A26 | 2-(4-{2-[(S)-2-methyl-1- azetidinyl]-6- (difluoromethyl)-5-ethyl- 4-pyrimidinyl}-1- pyrazolyl)-1-(1- piperazinyl)-1-ethanone | 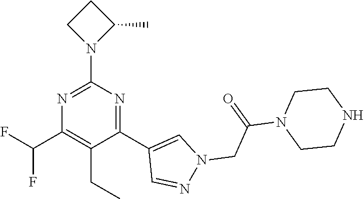 [SMILES: CCc1c(-c2cnn(CC(=O)N3CCNCC3)c2)nc(N2CC[C@@H]2C)nc1C(F)F] |
| A27 | (2S,3R)-1-{5-fluoro-4-[1- (1-methyl-3-azetidinyl)-4- pyrazolyl]-6- (trifluoromethyl)-2- pyrimidinyl}-2-methyl-3- azetidinol | 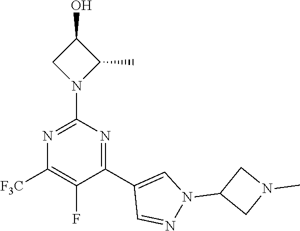 [SMILES: C[C@H]1[C@H](O)CN1c1nc(-c2cnn(C3CN(C)C3)c2)c(F)c(C(F)(F)F)n1] |
| A28 | 2-(4-{2-[(R)-2- (trifluoromethyl)-1- azetidinyl]-5-methoxy-6- (trifluoromethyl)-4- pyrimidinyl}-1-pyrazolyl)- 1-(1-piperazinyl)-1- ethanone | 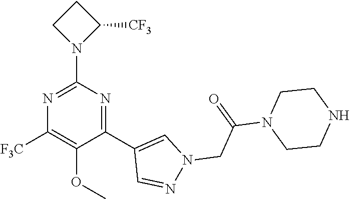 [SMILES: COc1c(-c2cnn(CC(=O)N3CCNCC3)c2)nc(N2CC[C@@H]2C(F)(F)F)nc1C(F)(F)F] |
| A29 | 2-(4-{2-[(R)-2- (trifluoromethyl)-1- azetidinyl]-5-chloro-6- (trifluoromethyl)-4- pyrimidinyl}-1-pyrazolyl)- 1-(1-piperazinyl)-1- ethanone | 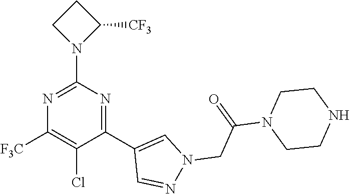 [SMILES: O=C(Cn1cc(-c2nc(N3CC[C@@H]3C(F)(F)F)nc(C(F)(F)F)c2Cl)cn1)N1CCNCC1] |
| A30 | 2-(4-{2-[(S)-2-methyl-1- azetidinyl]-5-cyclopropyl- 6-(trifluoromethyl)-4- pyrimidinyl}-1-pyrazolyl)- 1-(1-piperazinyl)-1- ethanone | 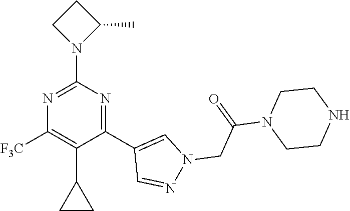 [SMILES: C[C@H]1CCN1c1nc(-c2cnn(CC(=O)N3CCNCC3)c2)c(C2CC2)c(C(F)(F)F)n1] |
| A31 | (2R,3R)-1-{4-[1-(3- azetidinyl)-4-pyrazolyl]-6- (trifluoromethyl)-2- pyrimidinyl}-2- (trifluoromethyl)-3- azetidinol | 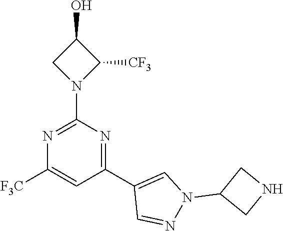 [SMILES: O[C@@H]1CN(c2nc(-c3cnn(C4CNC4)c3)cc(C(F)(F)F)n2)[C@H]1C(F)(F)F] |
| A32 | 2-(4-{2-[(S)-2-methyl-1- azetidinyl]-5-fluoro-6- (trifluoromethyl)-4- pyrimidinyl}-1-pyrazolyl)- 1-(1-piperazinyl)-1- ethanone | 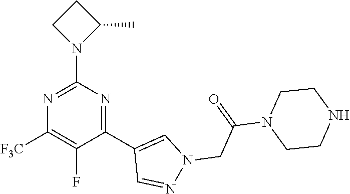 [SMILES: C[C@H]1CCN1c1nc(-c2cnn(CC(=O)N3CCNCC3)c2)c(F)c(C(F)(F)F)n1] |
| A33 | 2-(4-{2-[(R)-2- (difluoromethyl)-1- azetidinyl]-6- (trifluoromethyl)-4- pyrimidinyl}-1-pyrazolyl)- 1-(1-piperazinyl)-1- ethanone | 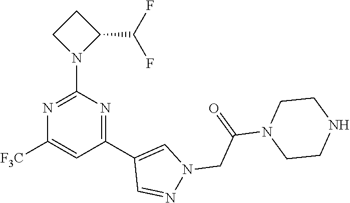 [SMILES: O=C(Cn1cc(-c2cc(C(F)(F)F)nc(N3CC[C@@H]3C(F)F)n2)cn1)N1CCNCC1] |
| A34 | (2S,3R)-1-{5-cyclopropyl- 4-[1-(1-methyl-3- azetidinyl)-4-pyrazolyl]-6- (trifluoromethyl)-2- pyrimidinyl}-2-methyl-3- azetidinol | 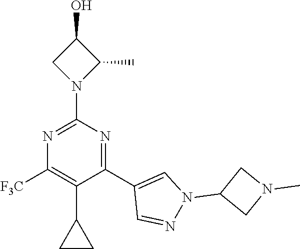 [SMILES: C[C@H]1[C@H](O)CN1c1nc(-c2cnn(C3CN(C)C3)c2)c(C2CC2)c(C(F)(F)F)n1] |
| A35 | (2S,3R)-1-{4-[1-(3- azetidinyl)-4-pyrazolyl]-6- (difluoromethyl)-5-methyl- 2-pyrimidinyl}-2-methyl- 3-azetidinol | 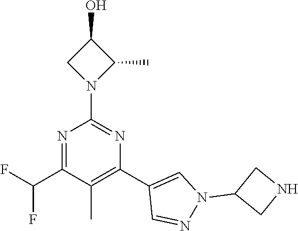 [SMILES: Cc1c(-c2cnn(C3CNC3)c2)nc(N2C[C@@H](O)[C@@H]2C)nc1C(F)F] |
| A36 | 2-(4-{2-[(R)-2- (difluoromethyl)-1- azetidinyl]-5-methoxy-6- (trifluoromethyl)-4- pyrimidinyl}-1-pyrazolyl)- 1-(1-piperazinyl)-1- ethanone | 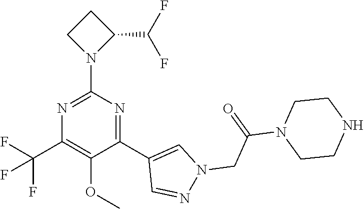 [SMILES: COc1c(-c2cnn(CC(=O)N3CCNCC3)c2)nc(N2CC[C@@H]2C(F)F)nc1C(F)(F)F] |
| A37 | 2-(4-{2-[(R)-2- (difluoromethyl)-1- azetidinyl]-5-chloro-6- (difluoromethyl)-4- pyrimidinyl}-1-pyrazolyl)- 1-(1-piperazinyl)-1- ethanone | 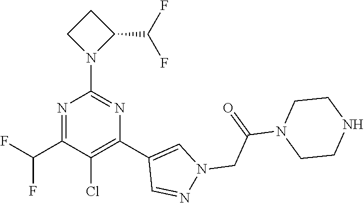 [SMILES: O=C(Cn1cc(-c2nc(N3CC[C@@H]3C(F)F)nc(C(F)F)c2Cl)cn1)N1CCNCC1] |
| A38 | 2-(4-{2-[(R)-2- (difluoromethyl)-1- azetidinyl]-5-chloro-6- (trifluoromethyl)-4- pyrimidinyl}-1-pyrazolyl)- 1-(1-piperazinyl)-1- ethanone | 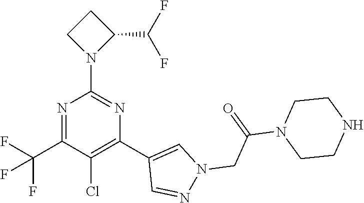 [SMILES: O=C(Cn1cc(-c2nc(N3CC[C@@H]3C(F)F)nc(C(F)(F)F)c2Cl)cn1)N1CCNCC1] |
| A39 | 2-(4-{2-[(R)-2- (difluoromethyl)-1- azetidinyl]-6- (difluoromethyl)-5-fluoro- 4-pyrimidinyl}-1- pyrazolyl)-1-(1- piperazinyl)-1-ethanone | 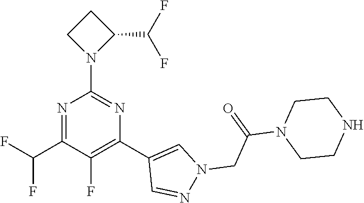 [SMILES: O=C(Cn1cc(-c2nc(N3CC[C@@H]3C(F)F)nc(C(F)F)c2F)cn1)N1CCNCC1] |
| A40 | 2-(4-{2-[(R)-2- (difluoromethyl)-1- azetidinyl]-6- (difluoromethyl)-5-ethyl- 4-pyrimidinyl}-1- pyrazolyl)-1-(1- piperazinyl)-1-ethanone | 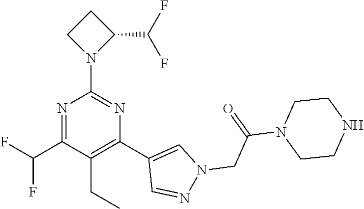 [SMILES: CCc1c(-c2cnn(CC(=O)N3CCNCC3)c2)nc(N2CC[C@@H]2C(F)F)nc1C(F)F] |
| A41 | 2-(4-{2-[(R)-2- (difluoromethyl)-1- azetidinyl]-5-ethyl-6- (trifluoromethyl)-4- pyrimidinyl}-1-pyrazolyl)- 1-(1-piperazinyl)-1- ethanone | 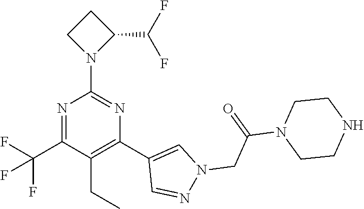 [SMILES: CCc1c(-c2cnn(CC(=O)N3CCNCC3)c2)nc(N2CC[C@@H]2C(F)F)nc1C(F)(F)F] |
| A42 | 2-(4-{2-[(R)-2- (difluoromethyl)-1- azetidinyl]-6- (difluoromethyl)-5-methyl- 4-pyrimidinyl}-1- pyrazolyl)-1-(1- piperazinyl)-1-ethanone | 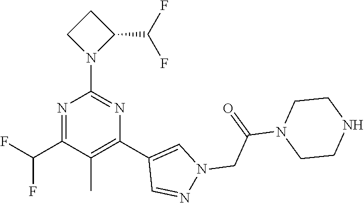 [SMILES: Cc1c(-c2cnn(CC(=O)N3CCNCC3)c2)nc(N2CC[C@@H]2C(F)F)nc1C(F)F] |
| A43 | 2-(4-{2-[(R)-2- (difluoromethyl)-1- azetidinyl]-5-methyl-6- (trifluoromethyl)-4- pyrimidinyl}-1-pyrazolyl)- 1-(1-piperazinyl)-1- ethanone | 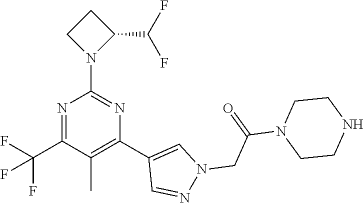 [SMILES: Cc1c(-c2cnn(CC(=O)N3CCNCC3)c2)nc(N2CC[C@@H]2C(F)F)nc1C(F)(F)F] |
| A44 | 2-(4-{2-[(R)-2- (difluoromethyl)-1- azetidinyl]-6- (difluoromethyl)-5- methoxy-4-pyrimidinyl}- 1-pyrazolyl)-1-(1- piperazinyl)-1-ethanone | 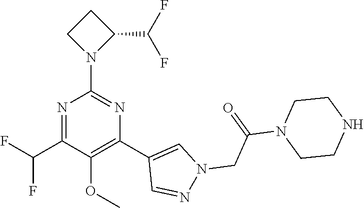 [SMILES: COc1c(-c2cnn(CC(=O)N3CCNCC3)c2)nc(N2CC[C@@H]2C(F)F)nc1C(F)F] |

36. (canceled)

37. (canceled)

38. (canceled)

39. (canceled)

40. (canceled)

41. A pharmaceutical composition comprising the compound or salt of claim 1 and a pharmaceutically acceptable excipient.

42. A method for inhibiting ketohexokinase (KHK) in a cell, comprising contacting the cell with the compound or salt claim 1.

43. A method for treating or preventing a disease or disorder in a subject, comprising administering to the subject a therapeutic amount of the compound or salt of claim 1.

44. (canceled)

45. (canceled)

46. (canceled)

47. (canceled)

48. (canceled)

49. (canceled)

50. (canceled)

51. (canceled)
